SEQUENCE LISTING (18 sequences)

SEQ ID NO: 1 - DNA, 1929 bp, Homo sapiens
  CDS (1)..(1929): fibronectin 1
atgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacggacagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctgtgagtatcccacccagaaaccttggatactga

SEQ ID NO: 2 - protein, 642 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTDSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPVSIPPRNLGY

SEQ ID NO: 3 - DNA, 1437 bp, Mus musculus
  CDS (1)..(1437): vitronectin
atggcacccctgaggccctttttcatactagccctggtggcatgggtttctctggctgaccaagagtcatgcaagggccgctgcactcagggtttcatggccagcaagaagtgtcagtgtgacgagctttgcacttactatcagagctgctgtgccgactacatggagcagtgcaagccccaagtaacgcggggggacgtgttcactatgccagaggatgattattggagctatgactacgtggaggagcccaagaacaataccaacaccggtgtgcaacccgagaacacctctccacccggtgacctaaatcctcggacggacggcactctaaagccgacagccttcctagatcctgaggaacagccaagcaccccagcgcctaaagtggagcaacaggaggagatcctaaggcccgacaccactgatcaagggacccctgagtttccagaggaagaactgtgcagtggaaagccctttgacgccttcacggatctcaagaatgggtccctctttgccttccgagggcagtaccgctgtgagctagatgagacggcagtgaggcctgggtaccccaaacttatccaagatgtctggggcattgagggccccatcgatgctgccttcactcgcatcaactgtcaggggaagacctacttgttcaagggtagtcagtactggcgctttgaggatggggtcctggaccctggttatccccgaaacatctccgaaggcttcagtggcataccagacaatgttgatgcagcgttcgcccttcctgcccaccgttacagtggccgggaaagggtctacttcttcaaggggaagcagtactgggagcacgaatttcagcagcaacccagccaggaggagtgcgaaggcagctctctgtcagccgtgtttgagcactttgccttgcttcagcgggacagctgggagaacattttcgaactcctcttctggggcagatcctctgatggagccagagaaccccaattcatcagccggaactggcatggtgtgccagggaaagtggacgctgctatggccggccgcatctacgtcactggctccttatcccactctgcccaagccaaaaaacagccgtctaagcgtagaagccgaaagcgctatcgttcacgccgagggcgtggccacagacgcagccagagctcgaactcccgtcgttcatcacgttcaatctggttctctttgttctccagcgaggagagtgggctaggaacctacaacaactatgattatgatatggactggcttgtacctgccacctgcgagcccattcagagcgtctatttcttctctggagacaaatactaccgagtcaaccttagaacccggcgagtggactctgtgaatcctccctacccacgctccattgctcagtattggctgggctgcccgacctctgagaagtag

SEQ ID NO: 4 - protein, 478 aa, Mus musculus
MAPLRPFFILALVAWVSLADQESCKGRCTQGFMASKKCQCDELCTYYQSCCADYMEQCKPQVTRGDVFTMPEDDYWSYDYVEEPKNNTNTGVQPENTSPPGDLNPRTDGTLKPTAFLDPEEQPSTPAPKVEQQEEILRPDTTDQGTPEFPEEELCSGKPFDAFTDLKNGSLFAFRGQYRCELDETAVRPGYPKLIQDVWGIEGPIDAAFTRINCQGKTYLFKGSQYWRFEDGVLDPGYPRNISEGFSGIPDNVDAAFALPAHRYSGRERVYFFKGKQYWEHEFQQQPSQEECEGSSLSAVFEHFALLQRDSWENIFELLFWGRSSDGAREPQFISRNWHGVPGKVDAAMAGRIYVTGSLSHSAQAKKQPSKRRSRKRYRSRRGRGHRRSQSSNSRRSSRSIWFSLFSSEESGLGTYNNYDYDMDWLVPATCEPIQSVYFFSGDKYYRVNLRTRRVDSVNPPYPRSIAQYWLGCPTSEK

SEQ ID NO: 5 - DNA, 9511 bp, Mus musculus
  CDS (121)..(9372): laminin-2 alpha chain
ggcacgagctgcaactccgtgggctccgggaggagtggatctgctccggccaggatgcctgcggccaccgccgggatcctcttgctcctgctcttggggacgctcgaaggctcccagactcagcggcgacagtcccaagcgcatcaacagagaggtttatttcctgctgtcctgaatcttgcttcgaatgcactcatcacaaccaatgctacatgtggggaaaaaggacccgagatgtactgcaagttggtggaacatgtccccgggcagcctgtgaggaaccctcagtgccgaatctgcaatcagaacagcagcaatccataccagaggcacccgattacgaatgctattgatggcaagaacacatggtggcagagtcccagtatcaagaatggagtggaataccattatgtgacaattactctggatttacagcaggtgttccagattgcctacgtaattgtgaaggcagccaattcccctcggcctggaaactggattttggaacgttccctggatgacgtggagtacaaaccctggcagtatcatgcggtgacagacacggagtgcctgaccctctacaatatctatccccgcactggaccaccatcctacgccaaagatgatgaggtcatctgcacttcattttattcgaagatccaccctttagaaaatggagagattcacatttctttgatcaatgggagaccaagtgctgatgacccctcccctgaactcctggaattcacctctgctcgctacattcgcctgagatttcagaggatccgcaccttgaatgcagacttgatgatgtttgctcacaaagaccccagagaaatcgatcccattgtcacacgaagatattactattctgtcaaggatatttcagttggcgggatgtgcatctgttatggtcatgcccgggcttgtccacttgaccctgcaacaaataaatcacgctgtgagtgtgaacataacacctgtggggaaagctgtgacaggtgctgtccaggattccatcagaagccttggagagctggaaccttcctcaccaagtctgagtgtgaagcatgcaattgtcacggaaaagctgaggaatgctattatgatgaaactgttgctagcagaaatctaagtttaaatatacatgggaagtacatcggagggggtgtgtgcatcaactgcacacataacacggctgggataaattgtgagacatgtgttgatggattcttcagacccaaaggggtgtcaccaaattatccaagaccatgccagccatgtcactgtgatccaactggctcccttagtgaagtctgtgtcaaagatgagaaatacgcccagcgagggttgaaacctggatcctgtcactgcaaaactggctttggaggcgtgaactgtgatcgctgtgtcaggggttaccatggttacccagactgccaaccctgtaactgtagtggcttggggagcacaaatgaggacccttgcgttgggccctgtagctgtaaggagaatgttgaaggtgaagactgtagtcgttgcaaatctggtttcttcaacttgcaagaagataatcagaaaggctgtgaggagtgtttctgttcaggagtatcaaacagatgtcagagttcctactggacctatgggaatattcaagacatgcgtggttggtatctcacagacctctctggccgcattcggatggctccccagcttgataaccctgactcacctcagcagatcagcatcagtaactctgaggcccggaaatccctgcttgatggttactactggagtgcaccgcctccatatctgggaaacagacttccagctgttgggggacagttgtcatttaccatctcatatgacctcgaagaagaggaagacgatacagaaaaactccttcagctgatgattatctttgagggaaatgacttaagaatcagcacagcgtataaggaggtgtacttagagccatctgaagaacacgttgaggaggtgtcactcaaagaagaggcctttactatacatggaacaaatttgccagtcactagaaaagatttcatgattgttctcacaaatttgggagagatccttatccaaatcacatacaacttagggatggacgccatcttcaggctgagttctgtcaatcttgaatctcctgtcccttatcctactgatagacgtattgcaactgatgtggaagtttgccagtgtccacctgggtacagtggcagctcttgtgaaacatgttggcctaggcaccgaagagttaacggcaccatttttggtggcatttgtgaaccatgtcagtgctttgctcatgcagaagcctgtgatgacatcacaggagaatgtctgaactgtaaggatcacacaggtgggccgtactgcaatgaatgtctccctggattctatggtgatcctactcgaggaagccctgaagactgtcagccctgtgcctgtccactcaatatcccatcaaataactttagtccaacatgccatttagaccggagtctgggattgatctgtgacgagtgtcctattgggtacacaggaccgcgctgtgagaggtgtgcagaaggctattttggacaaccttccgtacctggaggatcatgtcagccatgccaatgcaatgacaaccttgactactccatccctggcagctgtgacagcctgtctggctcctgtctgatttgtaagccaggtacaacaggccggtactgtgagctctgtgctgatgggtattttggagacgcggttaatacaaagaactgtcaaccatgccgttgtgatatcaatggctccttctcagaggattgtcacacaagaactgggcaatgtgagtgcagacccaatgttcaggggcggcactgtgacgagtgtaagcctgaaacctttggcctgcaactgggaaggggttgtctgccctgcaactgcaattcttttgggtctaagtcctttgactgtgaagcaagtgggcagtgctggtgccagcctggagtagcagggaagaaatgtgaccgttgtgcccatggctacttcaacttccaagaaggaggctgcatagcttgtgactgttctcatctgggcaacaactgtgacccaaaaactggccaatgcatttgcccacccaataccactggagaaaagtgttctgagtgtcttcccaacacctggggtcacagcattgtcaccggctgtaaggtttgtaactgcagcactgtggggtccttggcttctcagtgcaatgtaaacacgggccagtgcagctgtcatccaaaattctctggtatgaaatgctcagagtgcagccgaggtcactggaactatcctctctgcactctatgtgactgcttccttccaggcacagatgccacgacttgtgatctggagactaggaaatgctcctgtagtgatcaaactggacagtgcagctgtaaggtgaatgtggaaggcgtccactgtgacaggtgccggcctggcaaatttggactagatgccaagaacccacttggctgcagcagctgctactgctttggagttactagtcaatgctctgaagcaaaggggctgatccgtacgtgggtgactttgagtgatgaacagaccattctacctctggtggatgaggccctgcagcacacgactaccaaaggcattgctttccagaaaccagagattgttgcaaagatggatgaagtcaggcaagagctccatttggaacctttttactggaaactcccacaacaatttgaagggaaaaagttgatggcttatggtggcaaactcaagtatgccatctattttgaggctcgggatgagacaggctttgccacatataaacctcaagttatcattcgaggtggaactcctactcatgctagaattattaccagacacatggctgcccctctcattggccagttgacacggcatgaaatagaaatgacagagaaagaatggaaatattatggtgatgatcctcgaatcagtagaactgtgacccgtgaagacttcttggatatactatatgatattcactatatccttatcaaggctacttatggaaacgttgtgagacaaagccgcatttctgaaatctccatggaagtagctgaaccaggacatgtattagcagggagcccaccagcacacttgatagaaagatgcgattgccctcctggctattctggcttgtcttgtgagacgtgtgcaccaggattttaccgacttcgttctgaaccaggtgggcggactcctggaccaaccttagggacctgtgttccctgccaatgtaatggacacagcagtcagtgtgatcctgagacctcagtatgccagaattgtcagcatcacactgctggtgacttctgtgagcgctgtgcccttggctactatggaatcgtcaggggattgccaaatgactgccaaccatgtgcttgtcctctgatttcgcccagcaacaatttcagcccctcttgtgtattggaaggtctggaagattaccgttgcaccgcctgcccaaggggctatgaaggacagtactgtgaaaggtgtgccccaggctatactggcagcccaagcagccccggaggctcctgccaagaatgtgagtgtgacccttatggctccctaccggttccctgtgaccgggtcacaggactctgcacgtgccgccctggagccacaggaaggaagtgtgatggctgcgagcactggcatgcacgcgagggtgcagagtgtgtcttttgtggagacgagtgtacaggccttcttcttggtgacctggctcgtctagagcagatgaccatgaacatcaacctcacgggcccactgcctgctccatataaaattctgtatggtcttgaaaatacaactcaggaactcaagcacctgctatcaccgcaacgggcaccagagaggctcattcagttggcagagggcaacgtgaacacacttgtgatggaaacaaatgagctgctaaccagagcaaccaaagtgacagcagatggtgagcaaacaggacaagatgctgagaggaccaactccagagcagaatccttggaagaattcattaaagggcttgtccaggatgctgaagccataaatgaaaaagctgtaaaactaaatgaaaccttaggaaatcaagataagacagcagagagaaacttggaggagcttcaaaaggaaatcgaccggatgctgaaggaactgagaagtaaagatcttcaaacacagaaggaagttgctgaggatgagctcgtggcagcagaaggccttctgaagagagtaaacaagctgtttggagagcccagagcccagaatgaagatatggaaaaggatctccagcagaaactggcagagtacaagaacaaacttgatgatgcttgggatctattgagagaagccactgataaaacccgagatgctaatcgtttgtctgctgccaatcaaaaaaacatgaccatactggagacaaagaaggaggctattgaaggtagcaaacgacaaatagagaacactttaaaggaaggcaatgacatccttgatgaagccaatcaactcttaggtgaaatcaactcagtcatagattatgtcgacgacattaaaactaagttgccaccaatgtccgaggagctgagtgacaaaatagatgacctcgcccaggaaataaaggacagaaggcttgctgagaaggtgttccaggctgagagccatgctgctcagctgaacgactcgtctgctgtacttgatggaatcctggatgaggctaagaacatctctttcaatgccacggcagccttcagagcttacagtaatattaaagactacattgatgaagctgagaaagtggccagagaagccaaagagcttgcccaaggggctacaaaactggcaacaagtcctcagggcttattaaaagaagatgccaaaggctcccttcagaaaagcttcaggatcctcaatgaagccaagaagctagcaaacgatgtgaaaggaaatcacaatgatctaaatgacctgaaaaccaggttagaaactgctgaccttagaaacagtggacttctaggagctctaaatgacaccatggacaagttatcagccattacaaatgacacggctgctaaactgcaggctgtcaaagagaaagccagagaagccaatgacacagcaaaagctgtcctggcccaggttaaggacctgcatcagaacctagatggcctgaagcaaaactacaataaactggcagacagcgtggccaaaacgaacgctgtggtgaaagatccttccaaaaacaaaatcattgcagatgcaggcacttccgtgagaaatctagaacaggaagctgaccggctaatcgacaaactcaagcccatcaaggagcttgaggacaacctaaagaaaaacatttctgaaataaaggaactgatcaaccaagctcggaaacaagctaactctatcaaagtatctgtttcttcgggaggtgactgtgttcggacatacaggccagaaatcaagaaaggaagctacaataacatcgttgtccatgtcaagaccgctgttgccgacaacctccttttttatcttggaagtgccaaatttattgactttcttgctatagaaatgcgcaaaggcaaagtcagcttcctctggattgttggctctggagttggccgagtagggtttccagacttgaccatcgacgactcctattggtaccgtattgaagcatcaagaacgggaagaaatggatctatttctgtgagagctttagatggacccaaagccagtatggtacccagcacctaccattcagtgtctcctcccgggtatactatcctagatgtggatgcaaatgcaatgctgtttgttggtggcctgaccggaaaaataaagaaggccgatgctgtacgtgtgatcaccttcaccggctgtatgggagaaacatactttgacaacaaacctataggtttatggaacttccgggagaaagaaggcgactgtaagggatgtactgtcagcccacaagtggaagatagtgaggggactattcagtttgatggtgaaggctatgcattagtgagccggcccatccgctggtaccccaacatctccacagtcatgttcaagttccggacattttcatcaagtgctctcctgatgtatcttgccacacgagacctgaaagatttcatgagtgtagagctcagtgatggacatgtgaaagtcagctatgacctgggctcaggaatgacttccgttgtcagcaatcaaaaccataatgatgggaaatggaaagcattcacgctgtcgcggattcagaaacaagccaacatatcgattgtcgacatcgattctaaccaggaggagaatgtagctacttcatcttctggaaacaactttggtcttgacttgaaagcagatgacaaaatatattttggtggcctgccaactctgagaaacttgagtatgaaagcaaggccagaagtcaatgtgaagaaatactccggctgcctcaaagatattgaaatttcaagaacaccttacaatatactcagcagccctgattatgttggtgtgaccaaaggctgttcactggagaatgttaatacagttagtttccccaagcctggttttgtggagcttgccgctgtgtctattgatgttggaacagaaatcaatctgtcctttagtaccaggaacgagtctgggatcattctcttgggaagtggagggacactcacaccacccaggagaaaacggagacaaaccacacaggcttattatgccatattcctcaacaagggccgcttggaagtgcatctctcctcggggacacggacaatgaggaaaattgtcatcaaaccggagccaaatttgtttcatgatgggagagaacattctgtccacgtagaaagaaccagaggcatcttcactgttcaaattgatgaagacagaagacatatccaaaacctgacagaggaacagcccatcgaagtgaaaaagctctttgtcgggggtgctcctcctgaatttcagccctccccactcaggaatattccggcctttcaaggctgtgtgtggaaccttgttattaactccatccccatggactttgcgcagcctatagccttcaaaaatgccgacattggtcgctgtacctatcaaaagccccgggaagatgagagtgaagcagttccagctgaagttattgtccagcctcagtcggtgcccacccctgccttccctttcccagtccccaccatggtgcatggcccttgtgttgcagaatcagaaccagctcttctgacagggagcaagcagtttgggctttccagaaacagccacattgcaattgtctttgatgacaccaaagttaaaaaccgcctcaccattgagctggaggtacgaactgaagctgaatcaggcttgctcttctacatgggtcggatcaatcatgctgattttggtactgttcagctgaggaatgggttcccgttcttcagttatgatttggggagtgggagcaccagaaccatgatccccacaaaaatcaacgatggtcagtggcacaagattaagattgtgagagtgaagcaggagggaattctttatgtggatgatgcctccagccaaaccatcagtcccaagaaagccgacatcctggatgtcggggggattctgtatgtcggtggattgccgatcaactataccacacgcagaattggtccagtgacttacagcctggatggctgtgttaggaatcttcacatggaacaagcccctgttgatctggaccagcctacctccagctttcacgttgggacatgctttgcgaatgcagagagtgggacttactttgatggaaccggttttggtaaagcagttggtgggttcatcgttggattggaccttcttgtggaatttgaattccgtaccacaagacccactggggtcctcctggggatcagcagtcagaagatggatggaatgggtattgaaatgatcgacgagaagcttatgttccacgtggataatggcgctggccgattcactgcaatttatgatgctgagatcccaggccacatgtgcaatggacagtggtataaagtcactgccaagaagatcaaaaaccgtcttgagctggtggtagatgggaaccaggtggatgcccagagcccaaactcagcatcgacatcagctgatacaaacgaccctgttttcgttggcggtttcccaggtggcctcaatcagtttggcctgaccaccaacattaggttccgaggctgcatccgatctctgaagctcaccaaaggcactgcaaaccgctggaggttaattttgccaaggccctggaactgaggggtgttcaacctgtatcatgcccgactacctaataaagatagttcaatcctgaggagaattcatcaaaacaagtatatcaagttaaacaatatacactcctatcatattaataaaactaatgtgcagcggccgc

SEQ ID NO: 6 - protein, 3084 aa, Mus musculus
QRRQSQAHQQRGLFPAVLNLASNALITTNATCGEKGPEMYCKLVEHVPGQPVRNPQCRICNQNSSNPYQRHPITNAIDGKNTWWQSPSIKNGVEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGESCDRCCPGFHQKPWRAGTFLTKSECEACNCHGKAEECYYDETVASRNLSLNIHGKYIGGGVCINCTHNTAGINCETCVDGFFRPKGVSPNYPRPCQPCHCDPTGSLSEVCVKDEKYAQRGLKPGSCHCKTGFGGVNCDRCVRGYHGYPDCQPCNCSGLGSTNEDPCVGPCSCKENVEGEDCSRCKSGFFNLQEDNQKGCEECFCSGVSNRCQSSYWTYGNIQDMRGWYLTDLSGRIRMAPQLDNPDSPQQISISNSEARKSLLDGYYWSAPPPYLGNRLPAVGGQLSFTISYDLEEEEDDTEKLLQLMIIFEGNDLRISTAYKEVYLEPSEEHVEEVSLKEEAFTIHGTNLPVTRKDFMIVLTNLGEILIQITYNLGMDAIFRLSSVNLESPVPYPTDRRIATDVEVCQCPPGYSGSSCETCWPRHRRVNGTIFGGICEPCQCFAHAEACDDITGECLNCKDHTGGPYCNECLPGFYGDPTRGSPEDCQPCACPLNIPSNNFSPTCHLDRSLGLICDECPIGYTGPRCERCAEGYFGQPSVPGGSCQPCQCNDNLDYSIPGSCDSLSGSCLICKPGTTGRYCELCADGYFGDAVNTKNCQPCRCDINGSFSEDCHTRTGQCECRPNVQGRHCDECKPETFGLQLGRGCLPCNCNSFGSKSFDCEASGQCWCQPGVAGKKCDRCAHGYFNFQEGGCIACDCSHLGNNCDPKTGQCICPPNTTGEKCSECLPNTWGHSIVTGCKVCNCSTVGSLASQCNVNTGQCSCHPKFSGMKCSECSRGHWNYPLCTLCDCFLPGTDATTCDLETRKCSCSDQTGQCSCKVNVEGVHCDRCRPGKFGLDAKNPLGCSSCYCFGVTSQCSEAKGLIRTWVTLSDEQTILPLVDEALQHTTTKGIAFQKPEIVAKMDEVRQELHLEPFYWKLPQQFEGKKLMAYGGKLKYAIYFEARDETGFATYKPQVIIRGGTPTHARIITRHMAAPLIGQLTRHEIEMTEKEWKYYGDDPRISRTVTREDFLDILYDIHYILIKATYGNVVRQSRISEISMEVAEPGHVLAGSPPAHLIERCDCPPGYSGLSCETCAPGFYRLRSEPGGRTPGPTLGTCVPCQCNGHSSQCDPETSVCQNCQHHTAGDFCERCALGYYGIVRGLPNDCQPCACPLISPSNNFSPSCVLEGLEDYRCTACPRGYEGQYCERCAPGYTGSPSSPGGSCQECECDPYGSLPVPCDRVTGLCTCRPGATGRKCDGCEHWHAREGAECVFCGDECTGLLLGDLARLEQMTMNINLTGPLPAPYKILYGLENTTQELKHLLSPQRAPERLIQLAEGNVNTLVMETNELLTRATKVTADGEQTGQDAERTNSRAESLEEFIKGLVQDAEAINEKAVKLNETLGNQDKTAERNLEELQKEIDRMLKELRSKDLQTQKEVAEDELVAAEGLLKRVNKLFGEPRAQNEDMEKDLQQKLAEYKNKLDDAWDLLREATDKTRDANRLSAANQKNMTILETKKEAIEGSKRQIENTLKEGNDILDEANQLLGEINSVIDYVDDIKTKLPPMSEELSDKIDDLAQEIKDRRLAEKVFQAESHAAQLNDSSAVLDGILDEAKNISFNATAAFRAYSNIKDYIDEAEKVAREAKELAQGATKLATSPQGLLKEDAKGSLQKSFRILNEAKKLANDVKGNHNDLNDLKTRLETADLRNSGLLGALNDTMDKLSAITNDTAAKLQAVKEKAREANDTAKAVLAQVKDLHQNLDGLKQNYNKLADSVAKTNAVVKDPSKNKIIADAGTSVRNLEQEADRLIDKLKPIKELEDNLKKNISEIKELINQARKQANSIKVSVSSGGDCVRTYRPEIKKGSYNNIVVHVKTAVADNLLFYLGSAKFIDFLAIEMRKGKVSFLWIVGSGVGRVGFPDLTIDDSYWYRIEASRTGRNGSISVRALDGPKASMVPSTYHSVSPPGYTILDVDANAMLFVGGLTGKIKKADAVRVITFTGCMGETYFDNKPIGLWNFREKEGDCKGCTVSPQVEDSEGTIQFDGEGYALVSRPIRWYPNISTVMFKFRTFSSSALLMYLATRDLKDFMSVELSDGHVKVSYDLGSGMTSVVSNQNHNDGKWKAFTLSRIQKQANISIVDIDSNQEENVATSSSGNNFGLDLKADDKIYFGGLPTLRNLSMKARPEVNVKKYSGCLKDIEISRTPYNILSSPDYVGVTKGCSLENVNTVSFPKPGFVELAAVSIDVGTEINLSFSTRNESGIILLGSGGTLTPPRRKRRQTTQAYYAIFLNKGRLEVHLSSGTRTMRKIVIKPEPNLFHDGREHSVHVERTRGIFTVQIDEDRRHIQNLTEEQPIEVKKLFVGGAPPEFQPSPLRNIPAFQGCVWNLVINSIPMDFAQPIAFKNADIGRCTYQKPREDESEAVPAEVIVQPQSVPTPAFPFPVPTMVHGPCVAESEPALLTGSKQFGLSRNSHIAIVFDDTKVKNRLTIELEVRTEAESGLLFYMGRINHADFGTVQLRNGFPFFSYDLGSGSTRTMIPTKINDGQWHKIKIVRVKQEGILYVDDASSQTISPKKADILDVGGILYVGGLPINYTTRRIGPVTYSLDGCVRNLHMEQAPVDLDQPTSSFHVGTCFANAESGTYFDGTGFGKAVGGFIVGLDLLVEFEFRTTRPTGVLLGISSQKMDGMGIEMIDEKLMFHVDNGAGRFTAIYDAEIPGHMCNGQWYKVTAKKIKNRLELVVDGNQVDAQSPNSASTSADTNDPVFVGGFPGGLNQFGLTTNIRFRGCIRSLKLTKGTANRWRLILPRPWN

SEQ ID NO: 7 - DNA, 5583 bp, Mus musculus
  CDS (42)..(5441): laminin, beta 2
ccacgcgtccgggacaccagcccagtacccacacggtcgggatggagtgggcctcaggagaaccagggaggggcaggcagggacagcctttgccatgggaacttcgcttgggcctacttctaagtgtgctggctgccacattggcccaggccccgtccttggatgtacctggctgttctcgaggaagctgctatccagccaccggtgacctgttggtgggccgtgcggacagactgacggcctcatccacgtgtggcttgcatagccctcaaccctactgtattgtcagtcacctgcaggacgaaaagaagtgtttcctgtgtgactcccgacgtcccttctctgctcgagacaacccaaatagtcatcggatccagaatgtagtcaccagctttgcgccacaacgccggacggcctggtggcaatcggagaacggggttccaatggtcaccatccaactggacctggaagctgagtttcatttcacccacctcattatgacgttcaagacgttccggcctgctgctatgctggtggagcgttctgcagactttggccgcacctggcacgtgtaccgatatttttcctatgactgcggggctgacttcccgggaatcccactggccccgccacgtcgctgggatgatgtagtgtgtgagtcccgctactcagaaatcgagccgtctacggaaggcgaggtcatctatcgtgtgctggaccctgctattcctatcccagacccctacagctcacggattcagaacctgttgaagatcaccaacctacgagtgaacttaacccggcttcacacactgggagacaacttgcttgacccacggagggagatccgggaaaaatactattatgctctctatgaacttgtcatccgtggcaactgcttctgctatggccacgcctcacagtgtgcgcctgcaccaggggcgccggcccatgctgagggcatggtacacggagcctgtatctgcaagcacaatactcgtggactcaactgtgagcagtgtcaggatttctatcaggaccttccctggcaccctgcagaggacggccatactcacgcctgtcggaagtgtgagtgcaacgggcatactcatagctgccactttgacatggctgtctacctggcatctggaaatgtaagtggaggcgtatgcgatgggtgtcagcacaacacagctgggcgccattgtgagttctgccggcccttcttctaccgtgaccccaccaaggacatgcgggacccagctgtgtgccgtccttgtgactgtgaccctatgggttctcaagatggtggtcgctgtgattctcatgatgaccctgtgctaggactggtctcaggccagtgtcgctgcaaagaacacgtggttggcactcgctgccagcaatgccgtgatggcttctttggacttagtgccagtgaccctcgagggtgccagcgttgccagtgtaattcacggggcacagtgcctgggagctccccttgtgactccagtagtggaacctgtttctgcaagcgtctggtgaccggacatggctgtgaccgctgtctgcctggccactggggcctgagccatgacctgctgggctgccgtccctgtgactgtgatgtgggcggtgccttggatcctcagtgtgatgaggccaccggtcagtgccgctgccgccaacacatgattgggcggcgctgcgaacaagtgcagcctggctacttccggccttttctggaccatttaacctgggaggctgaggctgcccaagggcaggggcttgaggtggtagagcggctggtgaccaaccgagagactccgtcctggactggcccaggctttgtgcggctgcgagaaggtcaggaagtggagttcctggtgacctctttgcctagggccatggactatgacctgctactgcgctgggagccccaggtccctgagcaatgggcagagctggaactgatggtgcagcgtccggggcctgtgtctgctcacagtccgtgcgggcatgtgctgcctaaggatgaccgcattcaggggatgcttcacccaaacaccaggtttttggtgtttcccagacctgtctgccttgagcctggcatctcctacaagctgaagctgaaactgatcggaacagggggacgagcccagcctgaaacctcctactctggattactcattgactcgctggtcctgcagccccacgtcttggtgctagagatgtttagtgggggcgatgctgctgctctggagcgccgtaccacctttgaacgctaccgctgccatgaggaaggtctgatgcccagcaaggcccctctatctgagacctgtgcccccctcctcatcagcgtgtccgccttgatctacaatggcgccttgccatgtcagtgtgaccctcaaggctcactgagttctgaatgcagtcctcacggtggccagtgccggtgcaaacctggagtggttggacgccgttgtgatgtctgtgctactggctactatggctttggccctgcaggctgtcaagcctgccagtgtagtcctgatggagcactcagtgccctctgtgaagggactagtggacagtgcccctgccgacctggtgcctttggtcttcgctgtgaccactgtcaacgtggccagtggggattccctaattgccggccgtgtgtctgcaatgggcgtgcggatgagtgtgatacccacacaggcgcttgcctgggctgccgtgattacacggggggcgagcactgtgaaaggtgcattgctggttttcatggggacccacggctgccatatgggggccagtgccggccttgtccctgccctgaaggccctgggagccagcgacactttgctacttcttgccaccgggatggatattcccagcaaattgtgtgccagtgtcgagaaggctacacagggcttcggtgtgaagcttgtgcccccgggcactttggggacccatcaaagccaggtggcaggtgccaactgtgtgagtgcagtggaaacattgatcccatggaccctgatgcctgtgatccccacacggggcaatgcttgcgttgtttacacaacacagaggggccccactgtggctattgcaagcctggcttccatgggcaagctgcccgacagagctgtcaccgctgtacctgcaaccttctgggcacagatcccaggcggtgcccatctaccgacctgtgccattgtgacccaagcactgggcagtgcccatgccttccccatgtccaaggcctcaactgtgaccattgtgcccccaacttttggaacttcaccagtggccgtggctgccagccttgtgcttgtcacccaagccgggccagaggccctacctgcaatgagttcacagggcagtgtcactgtcatgctggctttggtgggaggacttgttctgagtgccaagagctctactggggagaccctggtctgcagtgccgtgcctgtgactgtgatcctagaggaatagacaaacctcagtgtcatcgttccacaggccactgtagctgccgcccaggcgtgtctggtgtgcgctgtgaccagtgtgctcgtggcttctcaggggtttttcctgcttgtcacccctgccacgcttgctttggagactgggatcgtgtggtacaggacctggctgctcggacgcggcgcctggagcagtgggctcaggagttgcagcaaacaggagtgctgggtgcctttgagagcagctttttgaacatgcaggggaagctaggcatggtgcaggccattatgagtgcccgcaatgcctcagccgcctctacggcgaagcttgtagaggccacagagggactacgtcatgaaatcgggaagaccaccgagcgcctgactcagttagaagcagagctaacagctgtgcaggatgagaacttcaatgccaaccatgcactcagtggtctggagagagacgggcttgcgcttaatctcaccctgaggcagctggatcagcatctggagatcctcaaacattcaaatttcttaggtgcctatgacagcatccgacatgcccacagccagtccacagaggcagagcgccgtgccaacgcctccacctttgcagtacccagccctgtgagcaactcagcagatacccggcgtcggacggaagtgctaatgggtgcccaaaaagaaaacttcaaccgccaacatttggccaaccagcaggcactgggacggctctctgcacatgcccacaccctgagcctgacgggcataaatgagttggtgtgtggggcaccaggggacgcaccctgtgccaccagcccttgtgggggtgccggatgtcgggatgaagatgggcagccccgttgtggtggcctcggttgcagtggggcagcagccacggcagatctagcgctgggccgggctcggcacacgcaggcagagctgcagcgggcactggtagaaggtggcggcatcctcagccgggtgtctgagactcgtcggcaggcagaagaggcacagcagcgagcacaggcagccctggacaaggctaatgcttccaggggccaggtggaacaggccaatcaggagcttcgagaacttatccagaatgtgaaagacttcctcagccaggagggagccgatcctgacagtattgaaatggtagcgactcgggtgctagacatctccatcccggcctcacccgagcagatccagcgcctagccagtgagattgcagaacgcgtccgaagcctggccgacgtggacacaatcctggcccataccatgggcgacgtgcgtcgggctgaacagctactgcaagatgcgcaccgggcacggagccgggccgagggtgagagacagaaggcagagacagtccaagcggcactggaggaggctcagagggcacaaggagctgctcagggtgccatctggggagcagtggttgacacacaaaacacagagcagaccctgcagcgggtccaggagaggatggcaggtgcagagaagtctctgaactctgccggtgagcgggctcggcaattagacgccctcctggaggccctgaaactgaaacgggcaggaaatagcctggcagcatctacagcggaagaaacagcaggcagtgcccagagccgtgccagggaggctgagaaacaactacgggaacaagtaggtgaccaataccaaacagtgagggcgttggcagagcggaaggctgaaggtgttctggctgcacaagccagggcagaacaactgcgggatgaggctcgggacctgttgcaggccgctcaggataagctgcagcggctacaggagctggagggcacatatgaggagaacgagcgtgcactggagggcaaagcggcccagctggatgggctggaagccaggatgcgcagtgtgctccaggccatcaacttgcaggtccagatctacaacacctgccagtgaccactccctagggcctagccttgtcgccaagcactgttctgcacacgatcgtccgcacattaaagagctcctggctagcaagagctttcaataaacctgtgtgaacctcaaaaaaaaaaaaaaaaaaaaaaaaaaaaaaaaa

SEQ ID NO: 8 - protein, 1799 aa, Mus musculus
MEWASGEPGRGRQGQPLPWELRLGLLLSVLAATLAQAPSLDVPGCSRGSCYPATGDLLVGRADRLTASSTCGLHSPQPYCIVSHLQDEKKCFLCDSRRPFSARDNPNSHRIQNVVTSFAPQRRTAWWQSENGVPMVTIQLDLEAEFHFTHLIMTFKTFRPAAMLVERSADFGRTWHVYRYFSYDCGADFPGIPLAPPRRWDDVVCESRYSEIEPSTEGEVIYRVLDPAIPIPDPYSSRIQNLLKITNLRVNLTRLHTLGDNLLDPRREIREKYYYALYELVIRGNCFCYGHASQCAPAPGAPAHAEGMVHGACICKHNTRGLNCEQCQDFYQDLPWHPAEDGHTHACRKCECNGHTHSCHFDMAVYLASGNVSGGVCDGCQHNTAGRHCEFCRPFFYRDPTKDMRDPAVCRPCDCDPMGSQDGGRCDSHDDPVLGLVSGQCRCKEHVVGTRCQQCRDGFFGLSASDPRGCQRCQCNSRGTVPGSSPCDSSSGTCFCKRLVTGHGCDRCLPGHWGLSHDLLGCRPCDCDVGGALDPQCDEATGQCRCRQHMIGRRCEQVQPGYFRPFLDHLTWEAEAAQGQGLEVVERLVTNRETPSWTGPGFVRLREGQEVEFLVTSLPRAMDYDLLLRWEPQVPEQWAELELMVQRPGPVSAHSPCGHVLPKDDRIQGMLHPNTRFLVFPRPVCLEPGISYKLKLKLIGTGGRAQPETSYSGLLIDSLVLQPHVLVLEMFSGGDAAALERRTTFERYRCHEEGLMPSKAPLSETCAPLLISVSALIYNGALPCQCDPQGSLSSECSPHGGQCRCKPGVVGRRCDVCATGYYGFGPAGCQACQCSPDGALSALCEGTSGQCPCRPGAFGLRCDHCQRGQWGFPNCRPCVCNGRADECDTHTGACLGCRDYTGGEHCERCIAGFHGDPRLPYGGQCRPCPCPEGPGSQRHFATSCHRDGYSQQIVCQCREGYTGLRCEACAPGHFGDPSKPGGRCQLCECSGNIDPMDPDACDPHTGQCLRCLHNTEGPHCGYCKPGFHGQAARQSCHRCTCNLLGTDPRRCPSTDLCHCDPSTGQCPCLPHVQGLNCDHCAPNFWNFTSGRGCQPCACHPSRARGPTCNEFTGQCHCHAGFGGRTCSECQELYWGDPGLQCRACDCDPRGIDKPQCHRSTGHCSCRPGVSGVRCDQCARGFSGVFPACHPCHACFGDWDRVVQDLAARTRRLEQWAQELQQTGVLGAFESSFLNMQGKLGMVQAIMSARNASAASTAKLVEATEGLRHEIGKTTERLTQLEAELTAVQDENFNANHALSGLERDGLALNLTLRQLDQHLEILKHSNFLGAYDSIRHAHSQSTEAERRANASTFAVPSPVSNSADTRRRTEVLMGAQKENFNRQHLANQQALGRLSAHAHTLSLTGINELVCGAPGDAPCATSPCGGAGCRDEDGQPRCGGLGCSGAAATADLALGRARHTQAELQRALVEGGGILSRVSETRRQAEEAQQRAQAALDKANASRGQVEQANQELRELIQNVKDFLSQEGADPDSIEMVATRVLDISIPASPEQIQRLASEIAERVRSLADVDTILAHTMGDVRRAEQLLQDAHRARSRAEGERQKAETVQAALEEAQRAQGAAQGAIWGAVVDTQNTEQTLQRVQERMAGAEKSLNSAGERARQLDALLEALKLKRAGNSLAASTAEETAGSAQSRAREAEKQLREQVGDQYQTVRALAERKAEGVLAAQARAEQLRDEARDLLQAAQDKLQRLQELEGTYEENERALEGKAAQLDGLEARMRSVLQAINLQVQIYNTCQ

SEQ ID NO: 9 - DNA, 5153 bp, Mus musculus
  CDS (1)..(1476): laminin 12 gamma 3 chain
atggctgtatccagggtcctgtccctcctggcaacggtggcatcgatggcgctggtgattcaggagacacacttcgcggcaggcgcggacatgggctcttgctacgacggtgtgggacgcgcacagcgctgtctgcctgagttcgagaacgcggcgttcggccgacgcgccgaggcctcccacacgtgcggacggcccccggaggacttctgtccacacgtgggggcaccaggggctgggctacagtgccagcgctgcgacgatgctgaccccggacgacgccacgacgcctcctacctcacagacttccacagccccgatgacagcacctggtggcagagcccatccatggccttcggggtgcagtaccccacctcggttaacctgaccttgagcttagggaaggcctatgagattacctatgtgaggctgaagttccacaccagtcgccctgagagttttgccatctacaagcgcacgtacgccagtggcccctgggagccctaccaatactacagtgcctcctgccagaaaacctatggccgtcctgagggccactacctgcgaccgggcgaggatgagagggtggccttctgcacctctgagttcagtgacatctcccccttgaacgggggcaacgtggccttctccaccctggaaggccgtcccagtgcctacaactttgaggagagccctgtgctgcaggagtgggtcaccagcactgacatcctgatctctctagatcggctcaacacgtttggggatgacatcttcaaggaccccagagtgctccagtcttactactacgctgtgtctgacttctctgtgggtggcaggtgcaaatgcaatggtcacgccagtgaatgcgaacccaatgcggctggtcagctggcttgccgctgtcagcacaacaccacaggagtggactgcgagcgttgtctgcccttcttccaggaccgtccgtgggcccgaggcaccgccgaggatgccaacgagtgtctgccctgcaactgcagtgggcactctgaggagtgcacgtttgacagggagctctatcggagcacaggccatggtgggcactgtcagcggtgccgtgaccacacaactgggccacactgtgagcgctgtgagaagaactactacagatggtccccgaagacaccatgccaaccctgtgactgccacccagcaggctctctgagtctccagtgtgacaactcaggcgtctgtccctgcaagcccacagtgactggctggaagtgtgaccgctgcctgcctggattccactcactcagtgagggcggctgcagaccctgtgcctgcaatgtcgccggcagcttgggcacctgtgacccccgcagtgggaactgtccctgcaaagagaatgtagaaggcagcctgtgtgacagatgccgccctgggacatttaacctgcagccccacaatccagtgggctgcagcagctgcttctgttatggccactccaaggtgtgttctcctgctgccgggttccaggaacaccacatccgctcagacttccgccatggagctggtggctggcagatcagaagcatgggagtgtccaagcgtcctctgcaatggagccagagtgggctcctcctgggcctgcgaggaggggaggaactctcagccccaaagaagttcctgggagaccagagactcagctatggacagccagtcatactgaccctccaagtaccccctggaggctccccacctcctattcagctgagactggagggagcaggcttggctctgtctctgaggccctccagtctacccagccctcaggacaccaggcagccaagacgagttcagctccagttcctcttgcaggagacttctgaggaggcagagtccccactgcccaccttccacttccagcgcctgctttccaatctgactgctctgagcatctggaccagtggccaaggaccgggccattctggccaagtgctcttgtgtgaagttcagctcacatcggcctggccccagcgtgagcttgcccctccagcctcttgggtggagacctgcttatgtccccagggatacacaggccagttctgtgaattctgtgctctgggatacaagagagaaatacctcatgggggtccctatgccaactgcattccctgcacctgcaaccagcatggcacctgtgaccccaacacagggatctgcctgtgtggccaccacaccgagggtccatcctgtgagcggtgcatgccaggtttctacggtaacgccttctcaggccgtgctgatgattgccagccctgtccgtgccctggccaatcagcctgtgcaaccatcccagagagtggagatgtggtgtgcacacactgccctcctggtcagagaggacgacgatgcgagagctgcgaagatggcttttttggggatcctctagggctctctggagctccccagccctgccgccgatgccagtgcagcgggaacgtggatctcaatgctgtgggcaactgtgatcctcattctggccactgcttgcgctgtctgtacaacacgacaggggcccactgcgagcactgtcgggagggtttctacgggagtgccgtggccacaaggcccgtggacaaatgtgctccctgcagctgtgacctgaggggctcagtcagtgagaagacctgcaaccctgtgactggccagtgtgtctgcctgccttatgtctccgggagggactgcagccgctgcagccctggcttctatgacctccagtctgggaggggctgccagagctgcaaatgtcacccacttggatccttggagaataagtgccaccccaagactggccagtgtccctgccgacctggtgtcactggccaagcctgtgacagatgccagctaggtttctttggcttctccatcaagggctgccgagactgtaggtgctccccattgggtgctgcctcatctcagtgccatgagaacagcacctgtgtgtgccggcccggctttgtgggctataaatgcgaccgctgccaggacaatttcttcctcgcggatggcgacacaggctgccaagagtgtcccacttgctatgccctagtgaaggaagaggcagccaagctgaaggccaggttgatgctgatggaggggtggcttcaaaggtctgactgtggtagcccctggggaccactagacattctgcagggagaagcccctctgggggatgtctaccaaggtcaccacctacttcaagagacccgggggaccttcctgcagcagatggtgggcctggaggattctgtgaaggccacttgggagcagttgcaggtgctgagagggcatgtacactgtgcccaggctggagctcagaagacctgcatccagctggcagagctggaggagacattgcagtcctcagaggaggaggtccttcgtgcagcctcagctctctcatttctggcaagtcttcagaaaggatccagcacacccaccaattggagtcacctggcatcagaggcccagatccttgccagaagccacagggacacggccaccaagatcgaagctacctcggaaagggccctgctcgcctccaacgccagctatgagctcctgaagctgatggaaggcagagtggcctcggaagcccagcaggaactggaggacaggtaccaggaggtgcaggcagctcagactgccctgggcatagctgtggcagaggcgctgcccaaagctgaaaaggcactggccacggtgaagcaagtcattggtgacgcagccccacatctaggcttgctggtcacccctgaagcaatgaacttccaagccaggggcctgagctggaaagtgaaggccctggagcagaagctggagcagaaggagcccgaggtgggccagtctgtgggagccctgcaggtggaggctggaagagccttggagaagatggagccctttatgcagctacgcaataagaccacagctgccttcacacgggcttcctcagctgtgcaagctgccaaggtgaccgtcataggagcagagaccctgctagctgacctagagggaatgaagctgaggtctcctctacccaaggagcaggcagcgctgaagaagaaagcaggcagcatcaggaccaggctcctggaggacacaaagaggaagaccaagcatgcagagaggatgctgggaaatgctgcctctctctcctccagcaccaagaagaaaagcaaagaagcagaactgatgtctaaggacaatgccaagctctccagagctttgctgagggaaggcaagcagggctaccgtcatgccagccgactcgccagccagacccaggccacactccgtcgggcctctcgcctgctgctgacctcagaagcacacaagcaggagctggaggaagctaaacaggtgacctctgggctgagcactgtggagcgccaggtccgagagtctcggatctccttggagaaggacaccaaggtcctgtcagagctgcttgtgaagctggggtccctgggtgtccaccaagcccctgctcagaccctgaacgagacccagcgggcactagaaagcttgaggctgcagctggattcccacggagccctgcatcacaaactgaggcagctggaggaagagtctgctcgacaggagctgcagattcagagctttgaggacgaccttgctgagatccgcgctgacaagcacaacttggagaccattctgagcagtctgccagagaactgtgccagctagaccctggtacaccctccccaccctgccgtttcctgtccactccctgtaggtgtcccaggtctgcctgtcgtatgttcacgtgaatgcttgtttgctggtgcatcttcggtctgagcaggagtgaatacatgctcacacctccacagatgaccctgtatgtagtcctcagtgtgtactctctaaacgtgcatcagcatacacaccccagtatttgcacatatgtgtatgtgatgcactgatgtgttaagaccacctgtgtgcatgcacacatatgagagtctagagctgtggagagcagtcctgagcttggcacatccacattctggtgggttcctgctatgaatatcctgcaggatgacacatctacacctcctcagaatcagggccaacaggtgtactcgagctga

SEQ ID NO: 10 - protein, 492 aa, Mus musculus
MAVSRVLSLLATVASMALVIQETHFAAGADMGSCYDGVGRAQRCLPEFENAAFGRRAEASHTCGRPPEDFCPHVGAPGAGLQCQRCDDADPGRRHDASYLTDFHSPDDSTWWQSPSMAFGVQYPTSVNLTLSLGKAYEITYVRLKFHTSRPESFAIYKRTYASGPWEPYQYYSASCQKTYGRPEGHYLRPGEDERVAFCTSEFSDISPLNGGNVAFSTLEGRPSAYNFEESPVLQEWVTSTDILISLDRLNTFGDDIFKDPRVLQSYYYAVSDFSVGGRCKCNGHASECEPNAAGQLACRCQHNTTGVDCERCLPFFQDRPWARGTAEDANECLPCNCSGHSEECTFDRELYRSTGHGGHCQRCRDHTTGPHCERCEKNYYRWSPKTPCQPCDCHPAGSLSLQCDNSGVCPCKPTVTGWKCDRCLPGFHSLSEGGCRPCACNVAGSLGTCDPRSGNCPCKENVEGSLCDRCRPGTFNLQPHNPVGCSSCFCY

SEQ ID NO: 11 - protein, 2265 aa, Bos taurus
QAQQIVQPQSPLTVSQSKPGCYDNGKHYQINQQWERTYLGSALVCTCYGGSRGFNCESKPEPEETCFDKYTGNTYRVGDTYERPKDSMIWDCTCIGAGRGRISCTIANRCHEGGQSYKIGDTWRRPHETGGYMLECVCLGNGKGEWTCKPIAEKCFDQAAGTSYVVGETWEKPYQGWMMVDCTCLGEGSGRITCTSRNRCNDQDTRTSYRIGDTWSKKDNRGNLLQCICTGNGRGEWKCERHTSLQTTSAGSGSFTDVRTAIYQPQPHPQPPPYGHCVTDSGVVYSVGMQWLKTQGNKQMLCTCLGNGVSCQETAVTQTYGGNSNGEPCVLPFTYNGKTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCVAYSQLRDQCIVDGITYNVNDTFHKRHEEGHMLNCTCFGQGRGRWKCDPVDQCQDSETRTFYQIGDSWEKYLQGVRYQCYCYGRGIGEWACQPLQTYPDTSGPVQVIITETPSQPNSHPIQWSAPESSHISKYILRWKPKNSPDRWKEATIPGHLNSYTIKGLRPGVVYEGQLISVQHYGQREVTRFDFTTTSTSPAVTSNTVTGETTPLSPVVATSESVTEITASSFVVSWVSASDTVSGFRVEYELSEEGDEPQYLDLPSTATSVNIPDLLPGRKYTVNVYEISEEGEQNLILSTSQTTAPDAPPDPTVDQVDDTSIVVRWSRPRAPITGYRIVYSPSVEGSSTELNLPETANSVTLSDLQPGVQYNITIYAVEENQESTPVFIQQETTGVPRSDKVPPPRDLQFVEVTDVKITIMWTPPESPVTGYRVDVIPVNLPGEHGQRLPVSRNTFAEVTGLSPGVTYHFKVFAVNQGRESKPLTAQQATKLDAPTNLQFINETDTTVIVTWTPPRARIVGYRLTVGLTRGGQPKQYNVGPAASQYPLRNLQPGSEYAVSLVAVKGNQQSPRVTGVFTTLQPLGSIPHYNTEVTETTIVITWTPAPRIGFKLGVRPSQGGEAPREVTSESGSIVVSGLTPGVEYVYTISVLRDGQERDAPIVKKVVTPLSPPTNLHLEANPDTGVLTVSWERSTTPDITGYRITTTPTNGQQGYSLEEVVHADQSSCTFENLSPGLEYNVSVYTVKDDKESVPISDTIIPAVPPPTDLRFTNVGPDTMRVTWAPPSSIELTNLLVRYSPVKNEEDVAELSISPSDNAVVLTNLLPGTEYLVSVSSVYEQHESIPLRGRQKTALDSPSGIDFSDITANSFTVHWIAPRATITGYRIRHHPENMGGRPREDRVPPSRNSITLTNLNPGTEYVVSIVALNSKEESLPLVGQQSTVSDVPRDLEVIAATPTSLLISWDAPAVTVRYYRITYGETGGSSPVQEFTVPGSKSTATISGLKPGVDYTITVYAVTGRGDSPASSKPVSINYRTEIDKPSQMQVTDVQDNSISVRWLPSSSPVTGYRVTTAPKNGPGPSKTKTVGPDQTEMTIEGLQPTVEYVVSVYAQNQNGESQPLVQTAVTTIPAPTNLKFTQVTPTSLTAQWTAPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAIPANGQTPIQRTIRPDVRSYTITGLQPGTDYKIHLYTLNDNARSSPVVIDASTAIDAPSNLRFLATTPNSLLVSWQPPRARITGYIIKYEKPGSPPREVVPRPRPGVTEATITGLEPGTEYTIQVIALKNNQKSEPLIGRKKTDELPQLVTLPHPNLHGPEILDVPSTVQKTPFITNPGYDTGNGIQLPGTSGQQPSLGQQMIFEEHGFRRTTPPTTATPVRHRPRPYPPNVNEEIQIGHVPRGDVDHHLYPHVVGLNPNASTGQEALSQTTISWTPFQESSEYIISCHPVGIDEEPLQFRVPGTSASATLTGLTRGATYNIIVEAVKDQQRQKVREEVVTVGNSVDQGLSQPTDDSCFDPYTVSHYAIGEEWERLSDSGFKLSCQCLGFGSGHFRCDSSKWCHDNGVNYKIGEKWDRQGENGQMMSCTCLGNGKGEFKCDPHEATCYDDGKTYHVGEQWQKEYLGAICSCTCFGGQRGWRCDNCRRPGAEPGNEGSTAHSYNQYSQRYHQRTNTNVNCPIECFMPLDVQADREDSRE

SEQ ID NO: 12 - DNA, 25 bp, Artificial
  primer
ggataaccgtattaccgccatgcat

SEQ ID NO: 13 - DNA, 46 bp, Artificial
  primer
ccctatctcggtctattcttttgcaaaagaatagaccgagataggg

SEQ ID NO: 14 - DNA, 1798 bp, Artificial
  pcr fragment
ggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagataggg

SEQ ID NO: 15 - DNA, 7391 bp, Artificial
  pTet-Off
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcactcgaggtcgagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgttgctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcacca

SEQ ID NO: 16 - DNA, 7391 bp, Artificial
  pTet-ON
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcatatgtctagattagataaaagtaaagtgattaacagcgcattagagctgcttaatgaggtcggaatcgaaggtttaacaacccgtaaactcgcccagaagcttggtgtagagcagcctacactgtattggcatgtaaaaaataagcgggctttgctcgacgccttagccattgagatgttagataggcaccatactcacttttgccctttaaaaggggaaagctggcaagattttttacgcaataacgctaaaagttttagatgtgctttactaagtcatcgcaatggagcaaaagtacattcagatacacggcctacagaaaaacagtatgaaactctcgaaaatcaattagcctttttatgccaacaaggtttttcactagagaacgcgttatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcactcgaggtcgagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgttgctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcacca

SEQ ID NO: 17 - protein, 5 aa, Artificial
  amino acid
IKVAV

SEQ ID NO: 18 - DNA, 3988 bp, Artificial
  pTRE-d2EGFP
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggaattcgagctcggtacccggggatcctctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2010/0144038 A1
(43) Pub. Date: Jun. 10, 2010

(54) Composition And Method For Increasing Efficiency Of Introduction Of Target Substance Into Cell

(76) Inventors: Masato Miyake, Amagasaki-shi (JP); Tomohiro Yoshikawa, Amagasaki-shi (JP); Eiichiro Uchimura, Amagasaki-shi (JP); Jun Miyake, Amagasaki-ken (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 10/594,349

(22) PCT Filed: Mar. 3, 2004
(86) PCT No.: PCT/JP04/02696

§ 371 Date: May 29, 2007

(30) Foreign Application Priority Data
Mar. 4, 2003 (JP) 2003-057869

(51) Int. Cl. C12N 15/09; C07K 14/00; C12N 5/06; C07K 14/78

(52) U.S. Cl. 435/455; 435/325; 530/350; 530/395; 530/396

(57) ABSTRACT

The present invention provides a method capable of improving the efficiency of introducing a target substance (e.g., DNA, polypeptides, sugars, or complexes thereof), which is difficult to introduce (particularly, transfect) into a cell in any circumstances. Particularly, the present invention provides a composition for increasing the efficiency of introducing a target substance into a cell, comprising (a) an actin acting substance. The present invention also provides a device and method using such a composition.

### TECHNICAL FIELD

The present invention relates to the field of cell biology. More particularly, the present invention relates to a compound, composition, device, method and system for increasing the efficiency of introducing a substance into a cell.

### BACKGROUND ART

Techniques for introducing a target substance (e.g., proteins, etc.) into cells (i.e., transfection, transformation, transduction, etc.) are generally used in a wide variety of fields, such as cell biology, genetic engineering, molecular biology, and the like.

Transfection is conducted to temporarily express a gene in cells, such as animal cells and the like, so as to observe an influence of the gene. Since the advent of the postgenome era, transfection techniques are frequently used to elucidate the functions of genes encoded by the genome.

Various techniques and agents used therein have been developed to achieve transfection. One of the techniques employs a cationic substance, such as a cationic polymer, a cationic lipid, or the like, and is widely used.

In many cases, however, use of conventional agents is not sufficient for transfection efficiency. No agent, which can be used either in solid phase or in liquid phase, has been conventionally developed. Therefore, there is a large demand for such an agent. Further, there is an increasing demand for a technique for efficiently introducing (e.g., transfecting, etc.) a target substance into cells or the like on a solid phase, such as microtiter plates, arrays, and the like.

The difficulty in transfecting cells or producing transgenic organisms hinders the progression of development of dominant negative screening in mammals. To overcome this problem, high-efficiency retrovirus transfection has been developed. Although this retrovirus transfection is potent, it is necessary to produce DNA to be packaged into viral intermediates, and therefore, the applicability of this technique is limited. Alternatively, high-density transfection arrays are being developed, but are not necessarily applicable to all cells. Various systems for liquid phase transfection have been developed. However, efficiency is low for adherent cells, for example. Thus, such techniques are not necessarily applicable to all cells.

Accordingly, a transfection system, which is applicable to all systems and all cells, has been desired in the art. Such a transfection system can be expected to be applied to large-scale high-throughput assays using, for example, microtiter plates, arrays, and the like, for various cells and experimentation systems. There is an increasing demand for such a transfection system.

### DISCLOSURE OF THE INVENTION

An object of the present invention is to provide a method for improving the efficiency of introducing (particularly, transfecting) target substances (e.g., DNA, polypeptides, sugars, or complexes thereof, etc.), which are conventionally difficult to introduce into cells via diffusion or hydrophobic interaction, in any circumstances.

The above-described object of the present invention was achieved by unexpectedly finding that a system using an actin acting substance can be used to dramatically increase the efficiency of introducing target substances into cells. This achievement is attributed in part to the unexpected finding that extracellular matrix proteins (e.g., fibronectin, vitronectin, laminin, etc.) act on actin.

Therefore, the present invention provides the following.

(1) A composition for increasing the efficiency of introducing a target substance into a cell, comprising:

(a) an actin acting substance.

(2) A composition according to item 1, wherein the actin acting substance may be an extracellular matrix protein or a variant or fragment thereof.
(3) A composition according to item 2, wherein the actin acting substance comprises at least one protein selected from the group consisting of fibronectin, laminin, and vitronectin, or a variant or fragment thereof.
(4) A composition according to item 1, wherein the actin acting substance comprises:

(a-1) a protein molecule comprising at least amino acids 21 to 241 of SEQ ID NO.: 11 constituting an Fn1 domain, or a variant thereof;

(a-2) a protein molecule having an amino acid sequence set forth in SEQ ID NO.: 2 or 11, or a variant or fragment thereof;

(b) a polypeptide having an amino acid sequence set forth in SEQ ID NO.: 2 or 11 having at least one mutation selected from the group consisting of at least one amino acid substitution, addition, and deletion, and having a biological activity;

(c) a polypeptide encoded by a splice or allelic mutant of a base sequence set forth in SEQ ID NO.: 1;

(d) a polypeptide being a species homolog of the amino acid sequence set forth in SEQ ID NO.: 2 or 11; or

(e) a polypeptide having an amino acid sequence having at least 70% identity to any one of the polypeptides (a-1) to (d), and having a biological activity.

(5) A composition according to item 1, wherein the Fn1 domain comprises amino acids 21 to 577 of SEQ ID NO.: 11.
(6) A composition according to item 1, wherein the protein molecule having the Fn1 domain is fibronectin or a variant or fragment thereof.
(7) A composition according to item 1, further comprising a gene introduction reagent.
(8) A composition according to item 1, wherein the gene introduction reagent is selected from the group consisting of cationic polymers, cationic lipids, and calcium phosphate.
(9) A composition according to item 1, further comprising a particle.
(10) A composition according to item 9, wherein the particle comprises gold colloid.
(11) A composition according to item 1, further comprising a salt.
(12) A composition according to item 11, wherein the salt is selected from the group consisting of salts contained in buffers and salts contained in media.
(13) A kit for increasing the efficiency of introducing a target substance into a cell, comprising:

(a) a composition comprising an actin acting substance; and

(b) a gene introduction reagent.

(14) A composition for increasing the efficiency of introducing a target substance into a cell, comprising:

A) a target substance; and

B) an actin acting substance.

(15) A composition according to item 14, wherein the target substance comprises a substance selected from the group consisting of DNA, RNA, polypeptides, sugars, and complexes thereof.
(16) A composition according to item 14, wherein the target substance comprises DNA encoding a gene sequence to be transfected.
(17) A composition according to item 16, further comprising a gene introduction reagent.
(18) A composition according to item 14, wherein the actin acting substance is an extracellular matrix protein or a variant or fragment thereof.
(19) A composition according to item 14, wherein the composition is provided in liquid phase.
(20) A composition according to item 14, wherein the composition is provided in solid phase.
(21) A device for introducing a target substance into a cell, comprising:

A) a target substance; and

B) an actin acting substance,

wherein the composition is fixed to a solid phase support.

(22) A device according to item 21, wherein the target substance comprises a substance selected from the group consisting of DNA, RNA, polypeptides, sugars, and complexes thereof.
(23). A device according to item 21, wherein the target substance comprises DNA encoding a gene sequence to be transfected.
(24) A device according to item 23, further comprising a gene introduction reagent.
(25) A device according to item 21, wherein the actin acting substance is an extracellular matrix protein or a variant or fragment thereof.
(26) A device according to item 21, wherein the staid phase support is selected from the group consisting of plates, microwell plates, chips, glass slides, films, beads, and metals.
(27) A device according to item 21, wherein the solid phase support is coated with a coating agent.
(28) A device according to item 27, wherein the coating agent comprises a substance selected from the group consisting of poly-L-lysine, silane, MAS, hydrophobic fluorine resins, and metals.
(29) A method for increasing the efficiency of introducing a target substance into a cell, comprising the steps of:

A) providing the target substance;

B) providing an actin acting substance; and

C) contacting the target substance and the actin acting substance with the cell.

(30) A method according to item 29, wherein the target substance comprises a substance selected from the group consisting of DNA, RNA, polypeptides, sugars, and complexes thereof.
(31) A method according to item 29, wherein the target substance comprises DNA encoding a gene sequence to be transfected.
(32) A method according to item 31, further comprising providing a gene introduction reagent, wherein the gene introduction reagent is contacted with the cell.
(33) A method according to item 29, wherein the actin acting substance is an extracellular matrix protein or a variant or fragment thereof.
(34) A method according to item 29, wherein the steps are conducted in liquid phase.
(35) A method according to item 29, wherein the steps are conducted in solid phase.
(36) A method for increasing the efficiency of introducing a target substance into a cell, comprising the steps of:

I) fixing a composition to a solid support, wherein the composition comprising:
A) a target substance; and
B) an actin acting substance; and

II) contacting the cell with the composition on the solid support.

(37) A method according to item 36, wherein the target substance comprises a substance selected from the group consisting of DNA, RNA, polypeptides, sugars, and complexes thereof.
(38) A method according to item 36, wherein the target substance comprises DNA encoding a gene sequence to be transfected.
(39) A method according to item 38, further comprising providing a gene introduction reagent, wherein the gene introduction reagent is contacted with the cell.
(40) A method according to item 39, further comprising forming a complex of the DNA and the gene introduction reagent after providing the gene introduction reagent, wherein after the forming step, the composition is provided by providing the actin acting substance.
(41) A method according to item 36, wherein the actin acting substance is an extracellular matrix protein or a variant or fragment thereof.

Hereinafter, the present invention will be described by way of preferred embodiments. It will be understood by those skilled in the art that the embodiments of the present invention can be appropriately made or carried out based on the description of the present specification and the accompanying drawings, and commonly used techniques well known in the art. The function and effect of the present invention can be easily recognized by those skilled in the art.

### BRIEF DESCRIPTION OF THE DRAWINGS

FIG. 1 shows the results of experiments in which various actin acting substances and HEK293 cells were used, where gelatin was used as a control. FIG. 1 shows an effect of each adhered substance (HEK cell) with respect to transfection efficiency. The HEK cells were transfected with pEGFP-N1 using an Effectene reagent.

FIG. 2 shows exemplary transfection efficiency when fibronectin fragments were used.

FIG. 3 shows exemplary transfection efficiency when fibronectin fragments were used.

FIG. 4 shows a summary of the results presented in FIGS. 2 and 3.

FIG. 5 shows the results of an example in which transfection efficiency was studied for various cells.

FIG. 6 shows the results of transfection when various plates were used.

FIG. 7 shows the results of transfection when various plates were used at a fibronectin concentration of 0, 0.27, 0.53, 0.8, 1.07, and 1.33 (μg/μL for each). FIG. 7 shows the influence of fibronectin concentration and the surface modification on the transfection of HEK293 cells. The data shows the average of 4 different squares.

FIG. 8 shows exemplary photographs showing cell adhesion profiles in the presence or absence of fibronectin.

FIG. 9 shows exemplary cross-sectional photographs of cell adhesion profiles in the presence or absence of fibronectin. Cross-sections of human mesenchymal stem cells (hMSC) were observed using a confocal laser scanning microscope. hMSCs were stained with SYTO61 (blue fluorescence) and Texas red-X phalloidin (red fluorescence) and fixed with 4% PFA. Blue fluorescence (nuclei: SYT061) and red fluorescence (nuclei: Texas red-X phalloidin) were obtained using a confocal laser microscope (LSM510, Carl Zeiss Co., Ltd., pin hole size=1.0, image interval=0.4).

FIG. 10 shows transition of nuclear surface area. Relative nuclear surface area was determined by cross-sections of hMSC observed with cofocal laser scanning microscopy. hMSC was fixed with 4% PFA.

FIG. 11 shows the results of an exemplary transfection experiment when a transfection array chip was constructed and used.

FIG. 12 shows exemplary contamination between each spot on an array.

FIGS. 13A and 13B show an experiment in which spatially-spaced DNA was caused to be taken into cells by the solid phase transfection of the present invention in Example 4. FIG. 13A schematically shows a method for producing a solid phase transfection array (SPTA). FIG. 13B shows the results of solid phase transfection. A HEK293 cell line was used to produce a SPTA. Green colored portions indicate transfected adherent cells. According to this result, the method of the present invention can be used to produce a group of cells separated spatially and transfected with different genes.

FIG. 13C shows a difference between conventional liquid phase transfection and SPTA.

FIGS. 14A and 14B shows the results of comparison of liquid phase transfection and SPTA. FIG. 14A shows the results of experiments where 5 cell lines were measured with respect to GFP intensity/mm². Transfection efficiency was determined as fluorescence intensity per unit area.

FIG. 14B shows fluorescence images of cells expressing EGFP corresponding to the data presented in FIG. 14A. White circular regions were regions in which plasmid DNA was fixed. In other regions, cells were also fixed in solid phase, however, cells expressing EGFP were not observed. The white bar indicates 500 μm.

FIG. 14C shows an exemplary transfection method of the present invention.

FIG. 14D shows an exemplary transfection method of the present invention.

FIGS. 15A and 15B show the results of coating a chip, where by cross contamination was reduced. FIGS. 15A and 15B show the results of liquid phase transfection and SPTA using HEK293 cells, HeLa cells, NIT3T3 cells (also referred to as “3T3”), HepG2 cells, and hMSCs. Transfection efficiency was represented by GFP intensity.

FIGS. 16A and 16B show cross contamination between each spot. A nucleic acid mixture containing fibronectin having a predetermined concentration was fixed to a chip coated with APS (γ-aminopropyl silane) or PLL (poly-L-lysine). Cell transfection was performed on the chip. Substantially no cross contamination was observed (upper and middle rows). In contrast, significant chip cross contamination of fixed nucleic acids was observed on a uncoated chip (lower row).

FIGS. 16C and 16D show a correlation relationship between the types of substances contained in a mixture used for fixation of nucleic acid and the cell adhesion rate. The graph of FIG. 16D shows an increase in the proportion of adherent cells over time. A longer time is required for cell adhesion when the slope of the graph is mild than when the slope of the graph is steep.

FIG. 17 shows the results of transfection using an RNAi transfection array of Example 5. Each reporter gene was printed on a solid phase substrate at a rate of 4 points per gene. The substrate was dried. For each transcription factor, siRNA (28 types) were printed onto coordinates at which reporter genes were printed, followed by drying. As a control, siRNA for EGFP was used. As a negative control, scramble RNA was used. Thereafter, LipofectAMINE2000 was printed onto the same coordinates of each gene, followed by drying. Thereafter, fibronectin solution was printed onto the same coordinates of each gene. HeLa-K cells were plated on the substrate, followed by culture for 2 days. Thereafter, images were taken using a fluorescence image scanner.

FIGS. 18A to 18E show the results of transfection using the RNAi transfection array of Example 5 for each cell. The fluorescence intensity of each reporter was quantified by image analysis, and thereafter, compared with the intensity of each reporter gene to which scramble RNA (negative control) was printed, thereby calculating the ratio. The results are shown for all reporters and all cells. D: pDsRed2-1 (promoterless vector: negative control to shRNA). G: pEGEP-N1 (green fluorescent protein expression vector: a target gene for shRNA used herein). sh: pPUR6iGFP272 (vector type RNAi suppressing the expression of EGFP gene). D+G, etc.: D was printed before G was printed (the order of printing is as written). D+G(7:3), etc.: the ratio of D to G, where the total amount of D and G genes was 2 μg and the ratio of the D gene to the G gene was 7:3.

FIG. 19 shows the results of transfection using an RNAi transfection array of Example 5. Each reporter gene expression unit PCR fragment was printed on a solid phase substrate at a rate of 4 points per gene. The substrate was dried. For each transcription factor, siRNA (28 types) were printed onto coordinates at which reporter genes were printed, followed by drying. As a control, siRNA for EGFP was used. As a negative control, scramble RNA was used. Thereafter, LipofectAMINE2000 was printed onto the same coordinates of each gene, followed by drying. Thereafter, fibronectin solution was printed onto the same coordinates of each gene. HeLa-K cells were plated on the substrate, followed by culture for 2 days. Thereafter, images were taken using a fluorescence image scanner.

FIGS. 20A to 20D show the results of transfection using the RNAi transfection array of Example 6 for each cell. The fluorescence intensity of each reporter was quantified by image analysis, and thereafter, compared with the intensity of each reporter gene to which scramble RNA (negative control) was printed, thereby calculating the ratio. The results are shown for all reporters and all cells.

FIG. 21 shows a structure of a PCR fragment obtained in Example 7.

FIG. 22 shows a structure of pEGFP-N1.

FIG. 23 shows the result of comparison of transfection efficiency of transfection microarrays using cyclic DNA and PCR fragments.

FIG. 24 shows changes when a tetracycline dependent promoter was used.

FIG. 25 shows the results of expression when a tetracycline dependent promoter and a tetracycline independent promoter were used.

### DESCRIPTION OF SEQUENCE LISTING

SEQ ID NO.: 1: a nucleic acid sequence of fibronectin (human)

SEQ ID NO.: 2: an amino acid sequence of fibronectin (human)

SEQ ID NO.: 3: a nucleic acid sequence of vitronectin (mouse)

SEQ ID NO.: 4: an amino acid sequence of vitronectin (mouse)

SEQ ID NO.: 5: a nucleic acid sequence of laminin (mouse α-chain)

SEQ ID NO.: 6: an amino acid sequence of laminin (mouse α-chain)

SEQ ID NO.: 7: a nucleic acid sequence of laminin (mouse β-chain)

SEQ ID NO.: 8: an amino acid sequence of laminin (mouse β-chain)

SEQ ID NO.: 9: a nucleic acid sequence of laminin (mouse γ-chain)

SEQ ID NO.: 10: an amino acid sequence of laminin (mouse γ-chain)

SEQ ID NO.: 11: an amino acid sequence of fibronectin (bovine)

SEQ ID NO.: 12: primer 1 used in Example 7

SEQ ID NO.: 13: primer 2 used in Example 7

SEQ ID NO.: 14: a PCR fragment obtained in a PCR reaction in Example 7

SEQ ID NO.: 15: pTet-Off used in Example 9

SEQ ID NO.: 16: pTet-On used in Example 9

SEQ ID NO.: 17: 5 amino acids of laminin

SEQ ID NO.: 18: pTRE-d2EGFP used in Example 9.

### BEST MODE FOR CARRYING OUT THE INVENTION

It should be understood throughout the present specification that articles for singular forms include the concept of their plurality unless otherwise mentioned. Therefore, articles or adjectives for singular forms (e.g., “a”, “an”, “the”, etc. in English; “ein”, “der”, “das”, “die”, etc. and their inflections in German; “un”, “une”, “le”, “la”, etc. in French; “un”, “una”, “el”, “la”, etc. in Spanish, and articles, adjectives, etc. in other languages) include the concept of their plurality unless otherwise specified. It should be also understood that terms as used herein have definitions ordinarily used in the art unless otherwise mentioned. Therefore, all technical and scientific terms used herein have the same meanings as commonly understood by those skilled in the art. Otherwise, the present application (including definitions) takes precedence.

### DEFINITION OF TERMS

Hereinafter, terms specifically used herein will be defined.

(Actin Acting Substances)

As used herein, the term “actin acting substance” refers to a substance which interacts directly or indirectly with actin within cells to alter the form or state of actin. Examples of such a substance include, but are not limited to, extracellular matrix proteins (e.g., fibronectin, vitronectin, laminin, etc.), and the like. Such actin acting substances include substances identified by the following assays. As used herein, interaction with actin is evaluated by visualizing actin with an actin staining reagent (Molecular Probes, Texas Red-X phalloidin) or the like, followed by microscopic inspection to observe and determine actin aggregation, actin reconstruction or an improvement in cellular outgrowth rate. Such evaluation may be performed quantitatively or qualitatively. Actin acting substances are herein utilized so as to increase transfection efficiency. An actin acting substance used herein is derived from any organism, including, for example, mammals, such as human, mouse, bovine, and the like.

As used herein, the term “extracellular matrix protein” refers to a protein constituting an “extracellular matrix”. As used herein, the term “extracellular matrix” (ECM) is also called “extracellular substrate” and has the same meaning as commonly used in the art, and refers to a substance existing between somatic cells no matter whether the cells are epithelial cells or non-epithelial cells. Extracellular matrices are involved in supporting tissue as well as in internal environmental structures essential for survival of all somatic cells. Extracellular matrices are generally produced from connective tissue cells. Some extracellular matrices are secreted from cells possessing basal membrane, such as epithelial cells or endothelial cells. Extracellular matrices are roughly divided into fibrous components and matrices filling there between. Fibrous components include collagen fibers and elastic fibers. A basic component of matrices is glycosaminoglycan (acidic mucopolysaccharide), most of which is bound to non-collagenous protein to form a polymer of a proteoglycan (acidic mucopolysaccharide-protein complex). In addition, matrices include glycoproteins, such as laminin of basal membrane, microfibrils around elastic fibers, fibers, fibronectins on cell surfaces, and the like. Particularly differentiated tissue has the same basic structure. For example, in hyaline cartilage, chondroblasts characteristically produce a large amount of cartilage matrices including proteoglycans. In bones, osteoblasts produce bone matrices which cause calcification. Examples of extracellular matrices for use in the present invention include, but are not limited to, collagen, elastin, proteoglycan, glycosaminoglycan, fibronectin, laminin, elastic fiber, collagen fiber, and the like. An extracellular matrix protein used in the present invention includes, for example, without limitation, fibronectin, vitronectin, laminin, and the like.

Examples of extracellular matrix proteins used in the present invention include, but are not limited to, at least one protein selected from the group consisting of fibronectin and its variants (e.g., pronectin F, pronectin L, pronectin Plus, etc.), laminin, and vitronectin, or a variant or fragment thereof. Such a fragment preferably has a molecular weight of, for example, at least 10 kDa. If a fragment has such a preferable molecular weight and has only 3 amino acids (e.g., a sequence of RGD), preferably at least 5 amino acids (IKVAV, SEQ ID NO.: 17), of an extracellular matrix protein sequence, the rest of the sequence may be arbitrarily changed as long as the capability of interacting with actin is retained.

As used herein, the term “Fn1 domain” typically refers to a sequence of fibronectin extending from the N terminus of its amino acid sequence and having a molecular weight of about 29 kDa (e.g., amino acids 21 to 241 of SEQ ID NO.: 11). In another embodiment, the domain may comprise a sequence of fibronectin extending from the N terminus of its amino acid sequence and having a molecular weight of about 72 kDa (e.g., amino acids 21 to 577 of SEQ ID NO.: 11). As an exemplary actin acting substance of the present invention, a polypeptide comprising the Fn1 domain or a variant thereof may be illustrated without limitation.

As used herein, the term “fibronectin” has the same meaning as that commonly understood by those skilled in the art, and refers to a protein which is conventionally categorized as an adhesion factor. Attention has been focused onto the cell adhesion function of fibronectin, so that fibronectin is being actively studied.

A gene encoding fibronectin herein comprises:

(a) a polynucleotide having a base sequence set forth in SEQ ID NO.: 1, or a fragment thereof;

(b) a polynucleotide encoding a polypeptide consisting of an amino acid sequence set forth in SEQ ID NO.: 2 or 11, or a fragment thereof;

(c) a polynucleotide encoding a variant polypeptide having the amino acid sequence set forth in SEQ ID NO.: 2 or 11 having at least one mutation selected from the group consisting of at least one amino acid substitution, addition, and deletion and having a biological activity;

(d) a polynucleotide which is a splice or allelic mutant of the base sequence set forth in SEQ ID NO.: 1;

(e) a polynucleotide encoding a polypeptide, which is a species homolog of the amino acid sequence set forth in SEQ ID NO.: 2 or 11; or

(g) a polynucleotide consisting of an amino acid sequence having at least 70% identity to any one of the polynucleotides (a) to (e) or a complementary sequence thereof, and encoding a polypeptide having a biological activity. Examples of biological activities include, but are not limited to, cell adhesion activity, heparin binding activity, collagen binding activity, actin acting activity first discovered in the present invention, and the like. A preferable biological activity is actin acting activity.

As used herein, “fibronectin” or “fibronectin polypeptide” comprises:

(a) a protein molecule having at least an amino acid sequence set forth in SEQ ID NO.: 2 or 11, or a variant thereof;

(b) a polypeptide having an amino acid sequence set forth in SEQ ID NO.: 2 or 11 having at least one mutation selected from the group consisting of at least one amino acid substitution, addition, and deletion, and having a biological activity;

(c) a polypeptide encoded by a splice or alleic mutant of a base sequence set forth in SEQ ID NO.: 1;

(d) a polypeptide being a species homolog of the amino acid sequence set fort in SEQ ID NO.: 2 or 11; or

(e) a polypeptide having an amino acid sequence having at least 70% identity to any one of the polypeptides (a) to (d), and having a biological activity.

As used herein, the term “vitronectin” has the same meaning as that commonly understood by those skilled in the art, and refers to a protein which is conventionally categorized into adhesion factors. Attention has been focused onto the cell adhesion function of vitronectin, so that vitronectin is being actively studied.

As used herein, a gene encoding vitronectin comprises:

(a) a polynucleotide having a base sequence set forth in SEQ ID NO.: 3, or a fragment thereof;

(b) a polynucleotide encoding a polypeptide consisting of an amino acid sequence set forth in SEQ ID NO.: 4, or a fragment thereof;

(c) a polynucleotide encoding a variant polypeptide having the amino acid sequence set forth in SEQ ID NO.: 4 having at least one mutation selected from the group consisting of at least one amino acid substitution, addition, and deletion, and having a biological activity;

(d) a polynucleotide which is a splice or alleic mutant of the base sequence set forth in SEQ ID NO.: 3;

(e) a polynucleotide encoding a species homolog of the polypeptide consisting of the amino acid sequence of SEQ ID NO.: 4;

(f) a polynucleotide hybridizable to any one of the polynucleotides (a) to (e) and encoding a polypeptide having a biological activity; or

(g) a polynucleotide consisting of a base sequence having at least 70% identity to any one of the polynucleotides (a) to (e) or a complementary sequence thereof, and encoding a polypeptide having a biological activity. Examples of biological activities include, but are not limited to, cell adhesion activity, heparin binding activity, collagen binding activity, complement activating activity, actin acting activity first discovered in the present invention, and the like. A preferable biological activity is actin acting activity.

As used herein, “vitronectin” or “vitronectin polypeptide” comprises:

(a) a protein molecule having at least an amino acid sequence set forth SEQ ID NO.: 4, or a variant thereof;

(b) a polypeptide having the amino acid sequence set forth in SEQ ID NO.: 4 having at least one mutation selected from the group consisting of at least one amino acid substitution, addition, and deletion, and having a biological activity;

(c) a polypeptide encoded by a splice or alleic mutant of a base sequence set forth in SEQ ID NO.: 3;

(d) a polypeptide which is a species homolog of the amino acid sequence set forth in SEQ ID NO.: 4; or

(e) a polypeptide having an amino acid sequence having at least 70% identity to any one of the polypeptides (a) to (d), and having a biological activity.

As used herein, the term “laminin” has the same meaning as that commonly understood by those skilled in the art, and refers to a protein which is conventionally categorized into adhesion factors. Attention has been focused onto the cell adhesion function of laminin, so that laminin is being actively studied.

As used herein, a gene encoding laminin comprises:

(a) polynucleotides having a base sequence set forth in SEQ ID NOS.: 5, 7, and 9, or a fragment thereof;

(b) polynucleotides encoding a polypeptide consisting of an amino acid sequence set forth in SEQ ID NOS.: 6, 8, and 10, or a fragment thereof;

(c) polynucleotides encoding a variant polypeptide having the amino acid sequence set forth in SEQ ID NOS.: 6, 8, and 10 having at least one mutation selected from the group consisting of at least one amino acid substitution, addition, and deletion, and having a biological activity;

(d) polynucleotides which are splice or alleic mutants of the base sequence set forth in SEQ ID NOS.: 5, 7, and 9;

(e) polynucleotides encoding a species homolog of a polypeptide consisting of the amino acid sequence set forth in SEQ ID NOS.: 6, 8, and 10;

(f) a polynucleotide hybridizable to any one of the polynucleotides (a) to (e) under stringent conditions, and having a biological activity; or

(g) a polynucleotide consisting of a base sequence having at least 70% identity to any one of the polynucleotides (a) to (e) or a complementary sequence thereof, and encoding a polypeptide having a biological activity. Examples of biological activities include, but are not limited to, cell adhesion activity, heparin binding activity, collagen binding activity, complement activating activity, actin acting activity first discovered in the present invention, and the like. A preferable biological activity is actin acting activity.

As used herein, “laminin” or “laminin polypeptide” comprises:

(a) protein molecules having at least an amino acid sequence set forth in SEQ ID NOS.: 6, 8 and 10, or a variant thereof;

(b) polypeptides having the amino acid sequence set forth in SEQ ID NOS.: 6, 8 and 10 having at least one mutation selected from the group consisting of at least one amino acid substitution, addition, and deletion, and having a biological activity;

(c) polypeptides encoded by a splice or alleic mutant of a base sequence set forth in SEQ ID NOS.: 5, 7 and 9;

(d) polypeptides which are a species homolog of the amino acid sequence set forth in SEQ ID NOS.: 6, 8 and 10; or

(e) a polypeptide having an amino acid sequence having at least 70% identity to any one of the polypeptides (a) to (d), and having a biological activity.

As used herein, the terms “cell adhesion molecule” and “adhesion molecule” are used interchangeably to refer to a molecule capable of mediating the joining of two or more cells (cell adhesion) or adhesion between a substrate and a cell. In general, cell adhesion molecules are divided into two groups: molecules involved in cell-cell adhesion (intercellular adhesion) (cell-cell adhesion molecules) and molecules involved in cell-extracellular matrix adhesion (cell-substrate adhesion) (cell-substrate adhesion molecules). In the method of the present invention, any molecule may be useful and may be effectively used. Therefore, cell adhesion molecules herein include a protein of a substrate and a protein of a cell (e.g., integrin, etc.) in cell-substrate adhesion. A molecule other than proteins falls within the concept of cell adhesion molecule as long as it can mediate cell adhesion.

For cell-cell adhesion, cadherin, a number of molecules belonging in an immunoglobulin superfamily (NCAML1, ICAM, fasciclin II, III, etc.), selectin, and the like are known, each of which is known to join cell membranes via a specific molecular reaction.

On the other hand, a major cell adhesion molecule functioning for cell-substrate adhesion is integrin, which recognizes and binds to various proteins contained in extracellular matrices. These cell adhesion molecules are all located on cell membranes and can be regarded as a type of receptor (cell adhesion receptor). Therefore, receptors present on cell membranes can also be used in a method of the present invention. Examples of such a receptor include, but are not limited to, α-integrin, β-integrin, CD44, syndecan, aggrecan, and the like. Techniques for cell adhesion are well known as described above and as described in, for example, “Saibogaimatorikkusu-Rinsho heno Oyo-[Extracellular matrix—Clinical Applications—], Medical Review.

It can be determined whether or not a certain molecule is a cell adhesion molecule, by an assay, such as biochemical quantification (an SDS-PAG method, a labeled-collagen method, etc.), immunological quantification (an enzyme antibody method, a fluorescent antibody method, an immunohistological study, etc.), a PCR method, a hybridization method, or the like, in which a positive reaction is detected. Examples of such a cell adhesion molecule include, but are not limited to, collagen, integrin, fibronectin, laminin, vitronectin, fibrinogen, an immunoglobulin superfamily member (e.g., CD2, CD4, CD8, ICM1, ICAM2, VCAM1), selectin, cadherin, and the like. Most of these cell adhesion molecules transmit into a cell an auxiliary signal for cell activation due to intercellular interaction as well as cell adhesion. Therefore, an adhesion factor for use in the present invention preferably transmits an auxiliary signal for cell activation into a cell. It can be determined whether or not such an auxiliary signal can be transmitted into a cell, by an assay, such as biochemical quantification (an SDS-PAG method, a labeled-collagen method, etc.), immunological quantification (an enzyme antibody method, a fluorescent antibody method, an immunohistological study, etc.), a PDR method, a hybridization method, or the like, in which a positive reaction is detected.

An example of a cell adhesion molecule is cadherin which is present in many cells capable of being fixed to tissue. Cadherin can be used in a preferred embodiment of the present invention. Examples of a cell adhesion molecule in cells of blood and the immune system which are not fixed to tissue, include, but are not limited to, immunoglobulin superfamily molecules (CD 2, LFA-3, ICAM-1, CD2, CD4, CD8, ICM1, ICAM2, VCAM1, etc.); integrin family molecules (LFA-1, Mac-1, gpIIbIIIa, p150, p95, VLA1, VLA2, VLA3, VLA4, VLA5, VLA6, etc.); selectin family molecules (L-selectin, E-selectin, P-selectin, etc.), and the like. Prior to the disclosure of the present invention, it had not been known that these substances increase transfection efficiency.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

### DEFINITION OF TERMS

Hereinafter, terms specifically used herein will be defined.

As used herein, the term “biological molecule” refers to a molecule relating to an organism and an aggregation thereof. As used herein, the term “biological” or “organism” refers to a biological organism, including, but being not limited to, an animal, a plant, a fungus, a virus, and the like. A biological molecule includes a molecule extracted from an organism and an aggregation thereof, though the present invention is not limited to this. Any molecule capable of affecting an organism and an aggregation thereof fall within the definition of a biological molecule. Therefore, low molecular weight molecules (e.g., low molecular weight molecule ligands, etc.) capable of being used as medicaments fall within the definition of biological molecule as long as an effect on an organism is intended. Examples of such a biological molecule include, but are not limited to, a protein, a polypeptide, an oligopeptide, a peptide, a polynucleotide, an oligonucleotide, a nucleotide, a nucleic acid (e.g., DNA such as cDNA and genomic DNA; RNA such as mRNA), a polysaccharide, an oligosaccharide, a lipid, a low molecular weight molecule (e.g., a hormone, a ligand, an information transmitting substance, a low molecular weight organic molecule, etc.), and a composite molecule thereof (glycolipids, glycoproteins, lipoproteins, etc.), and the like. A biological molecule may include a cell itself or a portion of tissue as long as it is intended to be introduced into a cell. Preferably, a biological molecule may include a nucleic acid (DNA or RNA) or a protein. In another preferred embodiment, a biological molecule is a nucleic acid (e.g., genomic DNA or cDNA, or DNA synthesized by PCR or the like). In another preferred embodiment, a biological molecule may be a protein.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a composite of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. A gene product, such as an extracellular matrix protein (e.g., fibronectin, etc.), is in the form of a typical polypeptide.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). A gene for an extracellular matrix protein (e.g., fibronectin, etc.) is in the form of a typical polynucleotide. A polynucleotide may be used for transfection.

As used herein, the term “nucleic acid molecule” is used interchangeably with “nucleic acid”, “oligonucleotide”, and “polynucleotide” and includes cDNA, mRNA, genomic DNA, and the like. As used herein, nucleic acid and nucleic acid molecule may be included by the concept of the term “gene”. A nucleic acid molecule encoding the sequence of a given gene includes “splice mutant (variant)”. Similarly, a particular protein encoded by a nucleic acid encompasses any protein encoded by a splice variant of that nucleic acid. “Splice mutants”, as the name suggests, are products of alternative splicing of a gene. After transcription, an initial nucleic acid transcript may be spliced such that different (alternative)) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, extracellular matrix proteins as used herein, which are useful as, for example, actin acting substances, may include their splice mutants.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, a fibronectin gene typically includes both a structural gene for fibronectin and a promoter for fibronectin. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context.

As used herein, the term “homology” in relation to a sequence (e.g., a nucleic acid sequence, an amino acid sequence, etc.) refers to the proportion of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, the term “similarity” in relation to a sequence (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using BLAST (sequence analyzing tool) with the default parameters.

As used herein, the term “amino acid” may refer to a naturally-occurring or nonnaturally-occurring amino acid as long as the object of the present invention is satisfied. The term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers. An embodiment using a D-isomer of an amino acid falls within the scope of the present invention. The term “nonnaturally-occurring amino acid” refers to an amino acid which is ordinarily not found in nature. Examples of nonnaturally-occurring amino acids include D-form of amino acids as described above, norleucine, para-nitrophenylalanine, homophenylalanine, para-fluorophenylalanine, 3-amino-2-benzyl propionic acid, D- or L-homoarginine, and D-phenylalanine. The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, the Fn1 domain used in the present invention may be a portion (domain) in an ortholog corresponding to a molecule (fibronectin) containing the domain.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” with respect to a polypeptide or polynucleotide refer to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%, as long as the same function is maintained. For this purpose, “about” may be herein put ahead of the numbers. However, it should be understood that the interpretation of numbers is not affected by the presence or absence of “about” in the present specification. In the present invention, a fragment preferably has a certain size or more (e.g., 5 kDa or more, etc.). Though not wishing to be bound by any theory, it is considered that a certain size is required for a fragment to act as an actin acting substance.

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T. “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 950.

As used herein, the term “salt” has the same meaning as that commonly understood by those skilled in the art, including both inorganic and organic salts. Salts are typically generated by neutralizing reactions between acids and bases. Salts include NaCl, K₂SO₄, and the like, which are generated by neutralization, and in addition, PbSO₄, ZnCl₂ and the like, which are generated by reactions between metals and acids. The latter salts may not be generated directly by neutralizing reactions, but may be regarded as a product of neutralizing reactions between acids and bases. Salts may be divided into the following categories: normal salts (salts without any H of acids or without any OH of bases, including, for example, NaCl, NH₄Cl, CH₃COONa, and Na₂CO₃) acid salts (salts with remaining H of acids, including, for example, NaHCO₃, KHSO₄, and CaHPO₄), and basic salts (salts with remaining OH of bases, including, for example, MgCl(OH) and CuCl(OH)). This classification is not very important in the present invention. Examples of preferable salts include salts constituting medium (e.g., calcium chloride, sodium hydrogen phosphate, sodium hydrogen carbonate, sodium pyruvate, HEPES, sodium chloride, potassium chloride, magnesium sulfide, iron nitrate, amino acids, vitamins, etc), salts constituting buffer (e.g., calcium chloride, magnesium chloride, sodium hydrogen phosphate, sodium chloride, etc.), and the like. These salts are preferable as they have a high affinity for cells and thus are better able to maintain cells in culture. These salts may be used singly or in combination. Preferably, these salts may be used in combination. This is because a combination of salts tends to have a higher affinity for cells. Therefore, a plurality of salts (e.g., calcium chloride, magnesium chloride, sodium hydrogen phosphate, and sodium chloride) are preferably contained in medium, rather than only NaCl or the like. More preferably, all salts for cell culture medium may be added to the medium. In another preferred embodiment, glucose may be added to medium.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane or the like or a microarray (microassay) in which genomic DNA is attached to a glass plate under stringent hybridization, PCR and in situ hybridization, and the like. It will be understood that Fn1 includes corresponding genes identified by such an electronic or biological search.

As used herein, the term “introduction” of a substance into a cell indicates that the substance enters the cell through the cell membrane. It can be determined whether or not the substance is successfully introduced into the cell, as follows. For example, the substance is labeled (e.g., with a fluorescent label, a chemoluminescent label, a phosphorescent label, a radioactive label, etc.) and the label is detected. Alternatively, changes in the cell, which are attributed to the substance (e.g., gene expression, signal transduction, events caused by binding to intracellular receptors, changes in metabolism, etc.), are measured physically (e.g., visual inspection, etc.), chemically (e.g., measurement of secreted substances, etc.), biochemically, or biologically. Therefore, the term “introduction” encompasses transfection, transformation, transduction and the like, which are usually called genetic manipulations as well as transferring of substances, such as proteins, into cells.

As used herein, the term “target substance” refers to a substance which is intended to be introduced into cells. Substances targeted by the present invention are substances which are not introduced under normal conditions. Therefore, substances which can be introduced into cells by diffusion or hydrophobic interaction under normal conditions, are not targeted in an important aspect of the present invention. Examples of substances which are not introduced into cells under normal conditions, include, but are not limited to, proteins (polypeptides), RNA, DNA, sugars (particularly, polysaccharides), and composite molecules thereof (e.g., glycoproteins, PNA, etc.), viral vectors, and other compounds.

As used herein, the term “device” refers to a part which can constitute the whole or a portion of an apparatus, and comprises a support (preferably, a solid phase support) and a target substance carried thereon. Examples of such a device include, but are not limited to, chips, arrays, microtiter plates, cell culture plates, Petri dishes, films, beads, and the like.

As used herein, the term “support” refers to a material which can fix a substance, such as a biological molecule. Such a support may be made from any fixing material which has a capability of binding to a biological molecule as used herein via covalent or noncovalent bond, or which may be induced to have such a capability.

Examples of materials used for supports include any material capable of forming a solid surface, such as, without limitation, glass, silica, silicon, ceramics, silicon dioxide, plastics, metals (including alloys), naturally-occurring and synthetic polymers (e.g., polystyrene, cellulose, chitosan, dextran, and nylon), and the like. A support may be formed of layers made of a plurality of materials. For example, a support may be made of an inorganic insulating material, such as glass, quartz glass, alumina, sapphire, forsterite, silicon oxide, silicon carbide, silicon nitride, or the like. A support may be made of an organic material, such as polyethylene, ethylene, polypropylene, polyisobutylene, polyethyleneterephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone, and the like. Also in the present invention, nitrocellulose film, nylon film, PVDF film, or the like, which are used in blotting, may be used as a material for a support. When a material constituting a support is in the solid phase, such as a support is herein particularly referred to as a “solid phase support”. A solid phase support may be herein in the form of a plate, a microwell plate, a chip, a glass slide, a film, beads, a metal (surface), or the like. A support may not be coated or may be coated.

As used herein, the term “liquid phase” has the same meanings as commonly understood by those skilled in the art, typically referring a state in solution.

As used herein, the term “solid phase” has the same meanings as commonly understood by those skilled in the art, typically referring to a solid state. As used herein, liquid and solid may be collectively referred to as a “fluid”.

As used herein, the term “contact” means that two substances (e.g., a compositions and a cell) are sufficiently close to each other so that the two substances interact with each other.

As used herein, the term “interaction” refers to, without limitation, hydrophobic interactions, hydrophilic interactions, hydrogen bonds, Van der Waals forces, ionic interactions, nonionic interactions, electrostatic interactions, and the like. Preferably, interaction may be a typical interaction, such as a hydrogen bond, a hydrophobic interaction, or the like, which takes place in organisms.

(Modification of Genes)

An actin acting substance used in the present invention is often used in the form of a gene product. It will be understood that such a gene product may be a variant thereof. Therefore, substances produced using the gene modification techniques described below can be used in the present invention.

In a given protein molecule, a given amino acid may be substituted with another amino acid in a structurally important region, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA sequence level, to produce a protein which maintains the original property after the substitution. Therefore, various modifications of peptides as disclosed herein and DNA encoding such peptides may be performed without clear losses of biological activity.

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within, more preferably within ±1, and even more preferably within ±0.5. It is understood in the art that such an amino acid substitution based on hydrophobicity is efficient. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of the conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse a-hemoglobin genes are orthologs, while the human a-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely, affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500 (1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using techniques well known in the art. Therefore, a peptide analog may be a polymer containing an amino acid analog.

Similarly, the term “polynucleotide analog” or “nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions maintains an intended function (e.g., the information transfer function of hormones and cytokines, etc.). For example, such a number may be one or several, and preferably within 20% or 10% of the full length, or no more than 100, no more than 50, no more than 25, or the like.

(Interactive Agent)

As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like.

As used herein, the term “agent” may refer to any substance or element as long as an intended object can be achieved (e.g., energy, etc.). Examples of such a substance include, but are not limited to, proteins, polypeptides, oligopeptides, peptides, polynucleotides, oligonucleotides, nucleotides, nucleic acids (e.g., DNA such as cDNA, genomic DNA and the like, or RNA such as mRNA, RNAi and the like), polysaccharides, oligosaccharides, lipids, low molecular weight organic molecules (e.g., hormones, ligands, information transduction substances, low molecular weight organic molecules, molecules synthesized by combinatorial chemistry, low molecular weight molecules usable as medicaments (e.g., low molecular weight molecule ligands, etc.), etc.), and composite molecules thereof. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

(Genetic Manipulation)

When genetic manipulation is mentioned herein, the term “vector” or “recombinant vector” refers to a vector transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for performing cloning is referred to as a “cloning vector”. Such a cloning vector ordinarily contains a multiple cloning site containing a plurality of restriction sites. Restriction enzyme sites and multiple cloning sites as described above are well known in the art and can be used as appropriate by those skilled in the art depending on the purpose in accordance with publications described herein (e.g., Sambrook et al., supra).

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers.

Examples of “recombinant vectors” for prokaryotic cells include, but are not limited to, pcDNA3(+), pBluescript-SK(+/−), pGEM-T, pEF-BOS, pEGFP, pHAT, pUC18, pFT-DEST™ 42GATEWAY (Invitrogen), and the like.

Examples of “recombinant vectors” for animal cells include, but are not limited to, pcDNAI/Amp, pcDNAI, pCDM8 (all, commercially available from Funakoshi), pAGE107 [Japanese Laid-Open Publication No. 3-229 (Invitrogen), pAGE103 [5. Biochem., 101, 1307 (1987)], pAMo, pAMoA [J. Biol. Chem., 268, 22782-22787 (1993)], a retrovirus expression vector based on a murine stem cell virus (MSCV), pEF-BOS, pEGFP, and the like.

Examples of recombinant vectors for plant cells include, but are not limited to, pPCVICEn4HPT, pCGN1548, pCGN1549, pBI221, pBI121, and the like.

As used herein, the term “terminator” refers to a sequence which is located downstream of a protein-encoding region of a gene and which is involved in the termination of transcription when DNA is transcribed into mRNA, and the addition of a poly-A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon. Examples of a promoter include, but are not limited to, a structural promoter, a specific promoter, an inductive promoter, and the like.

As used herein, the term “enhancer” refers to a sequence which is used so as to enhance the expression efficiency of a gene of interest. One or more enhancers may be used, or no enhancer may be used.

As used herein, the term “silencer” refers to a sequence which has a function of suppressing and arresting the expression of a gene. Any silencer which has such a function may be herein used. No silencer may be used.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene introduction & Expression Analysis”, Yodo-sha, 1997; and the like. Gene introduction can be confirmed by method as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like), a lipofection method, a spheroplast method (Proc. Natl. Acad. Sci. USA, 84, 1929 (1978)), a lithium acetate method (J. Bacteriol., 153, 163 (1983); and Proc. Natl. Acad. Sci. USA, 75, 1929 (1978)), and the like.

As used herein, the term “gene introduction reagent” refers to a reagent which is used in a gene introduction method so as to enhance introduction efficiency. Examples of such a gene introduction reagent include, but are not limited to, cationic polymers, cationic lipids, polyamine-based reagents, polyimine-based reagents, calcium phosphate, and the like. Specific examples of a reagent used in transfection include reagents available from various sources, such as, without limitation, Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE2000Reagent (11668-019, Invitrogen corporation, CA), JetPEI (×4) conc. (101-30, Polyplus-transfection, France) and ExGen 500 (R0511, Fermentas Inc., MD), and the like.

As used herein, “instructions” describe a method for introducing a target substance according to the present invention for users (e.g., researchers, laboratory technicians, medical doctors, patients, etc.). The instructions describe a statement indicating a method for using a composition of the present invention, or the like. The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions are a so-called package insert in the case of medicaments or a manual in the case of experimental reagents, and are typically provided in paper media. The instructions are not so limited and may be provided in the form of electronic media (e.g., web sites, electronic mails, and the like provided on the internet).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like. Alternatively, a cell separated from a naturally-occurring product may be used in the present invention.

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon cancer cell line, and the like.

The mouse myeloma cell includes ps20, NSO, and the like. The rat myeloma cell includes YB2/0 and the like. A human embryo kidney cell includes HEK293 (ATCC: CRL-1573) and the like. The human leukemic cell includes BALL-1 and the like. The African green monkey kidney cell includes COS-1, COS-7, and the like. The human colon cancer cell line includes, but is not limited to, HCT-15, human neuroblastoma SK-N-SH, SK-N-SH-5Y, etc.), mouse neuroblastoma (e.g., etc.), and the like. Alternatively, primary culture cells may be used in the present invention.

Examples of plant cells used herein in genetic manipulation include, but are not limited to, calluses or a part thereof, suspended culture cells, cells of plants in the families of Solanaceae, Poaceae, Brassicaceae, Rosaceae, Leguminosae, Cucurbitaceae, Lamiaceae, Liliaceae, Chenopodiaceae and Umbelliferae, and the like.

Gene expression (e.g., mRNA expression, polypeptide expression) may be “detected” or “quantified” by an appropriate method, including mRNA measurement and immunological measurement method. Examples of molecular biological measurement methods include Northern blotting methods, dot blotting methods, PCR methods, and the like. Examples of immunological measurement method include ELISA methods, RIA methods, fluorescent antibody methods, Western blotting methods, immunohistological staining methods, and the like, where a microtiter plate may be used. Examples of quantification methods include ELISA methods, RIA methods, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat. Genet. 2002 December; 32 Suppl:526-32. Examples of methods for analyzing gene expression include, but are not limited to, RT-PCR methods, RACE methods, SSCP methods, immunoprecipitation methods, two-hybrid systems, in vitro translation methods, and the like in addition to the above-described techniques. Other analysis methods are described in, for example, “Genome Analysis Experimental Method, Yusuke Nakamura's Lab-Manual, edited by Yusuke Nakamura, Yodo-sha (2002), and the like. All of the above-described publications are herein incorporated by reference.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

As used herein, the term “expression level” refers to the amount of a polypeptide or mRNA expressed in a subject cell. The term “expression level” includes the level of protein expression of a polypeptide evaluated by any appropriate method using an antibody, including immunological measurement methods (e.g., an ELISA method, an RIA method, a fluorescent antibody method, a Western blotting method, an immunohistological staining method, and the like, or the mRNA level of expression of a polypeptide evaluated by any appropriate method, including molecular biological measurement methods (e.g., a Northern blotting method, a dot blotting method, a PCR method, and the like). The term “change in expression level” indicates that an increase or decrease in the protein or mRNA level of expression of a polypeptide evaluated by an appropriate method including the above-described immunological measurement method or molecular biological measurement method.

Therefore, as used herein, the term “reduction” of “expression” of a gene, a polynucleotide, a polypeptide, or the like indicates that the level of expression is significantly reduced in the presence of or under the action of the agent of the present invention as compared to when the action of the agent is absent. Preferably, the reduction of expression includes a reduction in the amount of expression of a polypeptide. As used herein, the term “increase” of “expression” of a gene, a polynucleotide, a polypeptide, or the like indicates that the level of expression is significantly increased by introduction of an agent related to gene expression into cells (e.g., a gene to be expressed or an agent regulating such gene expression) as compared to when the action of the agent is absent. Preferably, the increase of expression includes an increase in the amount of expression of a polypeptide. As used herein, the term “induction” of “expression” of a gene indicates that the amount of expression of the gene is increased by applying a given agent to a given cell. Therefore, the induction of expression includes allowing a gene to be expressed when expression of the gene is not otherwise observed, and increasing the amount of expression of the gene when expression of the gene is observed.

As used herein, the term “specifically expressed” in relation to a gene indicates that the gene is expressed in a specific site or for a specific period of time, at a level different from (preferably higher than) that in other sites or for other periods of time. The term “specifically expressed” indicates that a gene may be expressed only in a given site (specific site) or may be expressed in other sites. Preferably, the term “specifically expressed” indicates that a gene is expressed only in a given site.

As used herein, the term “biological activity” refers to activity possessed by an agent (e.g., a polynucleotide, a protein, etc.) within an organism, including activities exhibiting various functions (e.g., transcription promoting activity, etc.). For example, when an actin acting substance interacts with actin, the biological activity thereof includes morphological changes in actin (e.g., an increase in cell extending speed, etc.) or other biological changes (e.g., reconstruction of actin filaments, etc.), and the like. Such a biological activity can be measured by, for example, visualizing actin with an actin staining reagent (Molecular Probes, Texas Red-X phalloidin) or the like, followed by microscopic inspection to observe aggregation of actin or cell extension. In another preferred embodiment, such a biological activity may be cell adhesion activity, heparin binding activity, collagen binding activity, or the like. Cell adhesion activity can be measured by, for example, measuring the rate of adhesion of disseminated cells to a solid phase, which is regarded as adhesion activity. Heparin binding activity can be measured by, for example, conducting affinity chromatography using heparin-fixed column or the like to determine whether or not a substance binds to the column. Collagen binding activity can be measured by, for example, conducting affinity chromatography using collagen-fixed column or the like to determine whether or not a substance binds to the column. For example, when a certain agent is an enzyme, the biological activity thereof includes enzymatic activity. In another example, when a certain agent is a ligand, the ligand binds to a corresponding receptor. Such binding activity is also biological activity. Such biological activity can be measured using techniques well known in the art (see Molecular Cloning, Current Protocols (supra), etc.).

As used herein, the term “particle” refers to a substance which has a certain hardness and a certain size or greater. A particle used in the present invention may be made of a metal or the like. Examples of particles used in the present invention include, but are not limited to, gold colloids, silver colloids, latex colloids, and the like.

As used herein, the term “kit” refers to a unit which typically has two or more sections, at least one of which is used to provide a component (e.g., a reagent, a particle, etc.). When materials are not provided after mixing and are preferably provided to prepare a composition immediately before use, a kit form is preferable. Such a kit preferably comprises instructions which describe how a component (e.g., a reagent, a particle, etc.) should be processed.

(Methods for Producing Polypeptides)

A transformant derived from a microorganism, an animal cell, or the like, which possesses a recombinant vector into which DNA encoding a polypeptide of the present invention is incorporated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium (e.g., RPMI1640 medium [The Journal of the American Medical Association, 199, 519 (1967)], Eagle's MEM medium [Science, 122, 501 (1952)], DMEM medium [Virology, 8, 396 (1959)], 199 medium [Proceedings of the Society for the Biological Medicine, 73, 1 (1950)] or these media supplemented with fetal bovine serum, or the like) as long as the medium contains a carbon source (e.g., carbohydrates (e.g., glucose, fructose, sucrose, molasses containing these, starch, starch hydrolysate, and the like), organic acids (e.g., acetic acid, propionic acid, and the like), alcohols (e.g., ethanol, propanol, and the like), etc.); a nitrogen source (e.g., ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), etc.), inorganic salts (e.g., potassium (I) phosphate, potassium (II) phosphate, magnesium phosphate, magnesium sulfate, sodium chloride, ferrous sulfate, manganous sulfate, copper sulfate, calcium carbonate, etc.), and the like which an organism of the present invention can assimilate and the medium allows efficient culture of the transformant. Culture is performed under aerobic conditions for shaking culture, deep aeration agitation culture, or the like. Culture temperature is preferably 15 to 40° C., culture time is ordinarily 5 hours to 7 days. The pH of culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain a soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When a polypeptide of the present invention is accumulated in a dissolved form within a transformant cell for producing the polypeptide, the culture is subjected to centrifugation to collect cells in the culture. The cells are washed, followed by pulverization of the cells using a ultrasonic pulverizer, a French press, MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When the polypeptide of the present invention has been expressed, and formed insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989), Genes Develop., 4, 1288 (1990)), a fusion protein of the polypeptide of the present invention with protein A is produced, followed by purification with affinity chromatography using immunoglobulin G.

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

Based on the amino acid information of a polypeptide as obtained above, the polypeptide can also be produced by a chemical synthesis method, such as the Fmoc method (fluorenylmethyloxycarbonyl method), the tBoc method (t-buthyloxycarbonyl method), or the like. The peptide can be chemically synthesized using a peptide synthesizer (manufactured by Advanced ChemTech, Applied Biosystems, Pharmacia Biotech, Protein Technology instrument, Synthecell-Vega, PerSeptive, Shimazu, or the like).

(Substrate/plate/chip/array)

As used herein, the term “plate” refers to a planar support onto which a molecule, such as an antibody or the like, may be fixed. In the present invention, a plate preferably comprises a glass substrate (base material), which has one side provided with a thin film made of a plastic, gold, silver or aluminum.

As used herein, the term “substrate” refers to a material (preferably solid material) with which a chip or array of the present invention is constructed. Therefore, a substrate is encompassed by the concept of a plate. Examples of materials for substrates include any solid materials to which a biological molecule used in the present invention is fixed via a covalent or noncovalent bond or which may be adapted to have such a property.

Examples of materials for plates and substrates include, but are not limited to, any material capable of forming solid surfaces, such as glass, silica, silicon, ceramics, silicon dioxide, plastics, metals (including alloys), naturally-occurring and synthetic polymers (e.g., polystyrene, cellulose, chitosan, dextran, and nylon), and the like. A substrate may be formed of a plurality of layers made of different materials. Examples of materials for plates and substrates include, but are not limited to, organic insulating materials, such as glass, quartz glass, alumina, sapphire, forsterite, silicon carbide, silicon oxide, silicon nitride, and the like. Examples of materials for plates and substrates also include, but are not limited to, organic materials, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone, and the like. A material preferable for a substrate varies depending on various parameters, such as measuring devices and the like, and can be selected as appropriate from the above-described various materials by those skilled in the art. For transfection arrays, glass slide is preferably. Preferably, the base material may be coated.

As used herein, the term “coating” in relation to a solid phase support or substrate refers to an act of forming a film of a material on a surface of the solid phase support or substrate, and also refers to a film itself. Coating is performed for various purposes, such as, for example, improvement in the quality of a solid phase support and substrate (e.g., elongation of life span, improvement in resistance to hostile environment, such as resistance to acids, etc.), an improvement in affinity to a substance integrated with a solid phase support or substrate, and the like. Such a substance used for coating is herein referred to as a “coating agent”. Various materials may be used for such coating, including, without limitation, biological substances (e.g., DNA, RNA, protein, lipid, etc.), polymers (e.g., poly-L-lysine, MAS (available from Matsunami Glass, Kishiwada, Japan), and hydrophobic fluorine resin), silane (APS (e.g., γ-aminopropyl silane, etc.)), metals (e.g., gold, etc.), in addition to the above-described solid phase support and substrate. The selection of such materials is within the technical scope of those skilled in the art and thus can be performed using techniques well known in the art. In one preferred embodiment, such a coating may be advantageously made of poly-L-lysine, silane (e.g., epoxy silane or mercaptosilane, APS (γ-aminopropyl silane), etc.), MAS, hydrophobic fluorine resin, a metal (e.g., gold, etc.). Such a material may be preferably a substance suitable for cells or objects containing cells (e.g., organisms, organs, etc.).

As used herein, the terms “chip” or “microchip” are used interchangeably to refer to a micro integrated circuit which has versatile functions and constitutes a portion of a system. Examples of a chip include, but are not limited to, DNA chips, protein chips, and the like.

As used herein, the terms “array” and “bioassay” are used interchangeably to refer to a substrate (e.g., a chip, etc.) which has a pattern of a composition containing at least one (e.g., 1000 or more, etc.) target substances (e.g., DNA, proteins, transfection mixtures, etc.), which are arrayed. Among arrays, patterned substrates having a small size (e.g., 10×10 mm, etc.) is particularly referred to as microarrays. The terms “microarray” and “array” are used interchangeably. Therefore, a patterned substrate having a larger size than that which is described above may be referred to as a microarray. For example, an array comprises a set of desired transfection mixtures fixed to a solid phase surface or a film thereof. An array preferably comprises at least 10² antibodies of the same or different types, more preferably at least 10³, even more preferably at least 10⁴, and still even more preferably at least 10⁵. These antibodies are placed on a surface of up to 125×80 mm, more preferably 10×10 mm. An array includes, but is not limited to, a 96-well microtiter plate, a 384-well microtiter plate, a microtiter plate the size of a glass slide, and the like. A composition to be fixed may contain one or a plurality of types of target substances. Such a number of target substance types may be in the range of from one to the number of spots, including, without limitation, about 10, about 100, about 500, and about 1,000.

As described above, any number of target substances (e.g., proteins, such as antibodies) may be provided on a solid phase surface or film, typically including no more than 10⁸ biological molecules per substrate, in another embodiment no more than 10⁷ biological molecules, no more than 10⁶ biological molecules, no more than 10⁵ biological molecules, no more than 10⁴ biological molecules, no more than 10³ biological molecules, or no more than 10² biological molecules. A composition containing more than 10⁸ biological molecule target substances may be provided on a substrate. In these cases, the size of a substrate is preferably small. Particularly, the size of a spot of a composition containing target substances (e.g., proteins such as antibodies) may be as small as the size of a single biological molecule (e.g., 1 to 2 nm order). In some cases, the minimum area of a substrate may be determined based on the number of biological molecules on a substrate. A composition containing target substances, which are intended to be introduced into cells, are herein typically arrayed on and fixed via covalent bonds or physical interaction to a substrate in the form of spots having a size of 0.01 mm to 10 mm.

“Spots” of biological molecules may be provided on an array. As used herein, the term “spot” refers to a certain set of compositions containing target substances. As used herein, the term “spotting” refers to an act of preparing a spot of a composition containing a certain target substance on a substrate or plate. Spotting may be performed by any method, for example, pipetting or the like, or alternatively, using an automatic device. These methods are well known in the art.

As used herein, the term “address” refers to a unique position on a substrate, which may be distinguished from other unique positions. Addresses are appropriately associated with spots. Addresses can have any distinguishable shape such that substances at each address may be distinguished from substances at other addresses (e.g., optically). A shape defining an address may be, for example, without limitation, a circle, an ellipse, a square, a rectangle, or an irregular shape. Therefore, the term “address” is used to indicate an abstract concept, while the term “spot” is used to indicate a specific concept. Unless it is necessary to distinguish them from each other, the terms “address” and “spot” may be herein used interchangeably.

The size of each address particularly depends on the size of the substrate, the number of addresses on the substrate, the amount of a composition containing target substances and/or available reagents, the size of microparticles, and the level of resolution required for any method used for the array. The size of each address may be, for example, in the range of from 1-2 nm to several centimeters, though the address may have any size suited to an array.

The spatial arrangement and shape which define an address are designed so that the microarray is suited to a particular application. Addresses may be densely arranged or sparsely distributed, or subgrouped into a desired pattern appropriate for a particular type of material to be analyzed.

Microarrays are widely reviewed in, for example, “Genomu Kino Kenkyu Purotokoru [Genomic Function Research Protocol] (Jikken Igaku Bessatsu [Special Issue of Experimental Medicine], Posuto Genomu Jidai no Jikken Koza 1 [Lecture 1 on Experimentation in Post-genome Era), “Genomu Ikagaku to korekarano Genomu Iryo [Genome Medical Science and Futuristic Genome Therapy (Jikken Igaku Zokan [Special Issue of Experimental Medicine]), and the like.

A vast amount of data can be obtained from a microarray. Therefore, data analysis software is important for administration of correspondence between clones and spots, data analysis, and the like. Such software may be attached to various detection systems (e.g., Ermolaeva O. et al., (1998) Nat. Genet., 20: 19-23). The format of database includes, for example, GATC (genetic analysis technology consortium) proposed by Affymetrix.

Micromachining for arrays is described in, for example, Campbell, S. A. (1996), “The Science and Engineering of Microelectronic Fabrication”, Oxford University Press; Zaut, P. V. (1996), “Micromicroarray Fabrication: a Practical. Guide to Semiconductor Processing”, Semiconductor Services; Madou, M. J. (1997), “Fundamentals of Microfabrication”, CRC1 5 Press; Rai-Choudhury, P. (1997), “Handbook of Microlithography, Micromachining, & Microfabrication: Microlithography”; and the like, portions related thereto of which are herein incorporated by reference.

(Cells)

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be either naturally-occurring cells or artificially modified cells (e.g., fusion cells, genetically modified cells, etc.). Examples of cell sources include, but are not limited to, a single-cell culture; the embryo, blood, or body tissue of normally-grown transgenic animal; a cell mixture of cells derived from normally-grown cell lines; and the like.

Cells used herein may be derived from any organism (e.g., any unicellular organisms (e.g., bacteria and yeast) or any multicellular organisms (e.g., animals (e.g., vertebrates and invertebrates), plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.). In one embodiment, cells derived from Primates (e.g., chimpanzee, Japanese monkey, human) are used. Particularly, without limitation, cells derived from a human are used.

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cell, tissue-specific stem cell, or somatic stem cell). A stem cell may be an artificially produced cell (e.g., fusion cells, reprogrammed cells, or the like used herein) as long as it can have the above-described abilities. Embryonic stem cells are pluripotent stem cells derived from early embryos. An embryonic stem cell was first established in 1981, which has been applied to production of knockout mice since 1989. In 1998, a human embryonic stem cell was established, which is currently becoming available for regenerative medicine. Tissue stem cells have a relatively limited level of differentiation unlike embryonic stem cells. Tissue stem cells are present in tissues and have an undifferentiated intracellular structure. Tissue stem cells have a higher nucleus/cytoplasm ratio and have few intracellular organelles. Most tissue stem cells have pluripotency, a long cell cycle, and proliferative ability beyond the life of the individual. As used herein, stem cells may be preferably embryonic stem cells, though tissue stem cells may also be employed depending on the circumstance.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they can achieve the intended treatment.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoietic stem cells, mesenchymal stem cells, and the like. Stem cells of mesoderm origin are mostly present in organs, including liver stem cells, pancreas stem cells, and the like. Somatic cells may be herein derived from any germ layer. Preferably, somatic cells, such as lymphocytes, spleen cells or testis-derived cells, may be used.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences naturally flanking the nucleic acid within an organism from which the nucleic acid is derived (i.e., sequences positioned at the 5′ terminus and the 3′ terminus of the nucleic acid).

As used herein, the term “established” in relation to cells refers to a state of a cell in which a particular property (pluripotency) of the cell is maintained and the cell undergoes stable proliferation under culture conditions. Therefore, established stem cells maintain pluripotency.

As used herein, the term “differentiated cell” refers to a cell having a specialized function and form (e.g., muscle cells, neurons, etc.). Unlike stem cells, differentiated cells have no or little pluripotency. Examples of differentiated cells include epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

(Medicaments and Cosmetics, and Therapy and Prevention Using the Same)

In another aspect, the present invention relates to medicaments (e.g., medicaments (vaccine, etc.), health foods, medicaments comprising a protein or lipid having reduced antigenicity, etc.), cosmetics, agricultural chemicals, foods, and the like, for introducing an effective ingredient into cells. Such medicaments and cosmetics may further comprise a pharmaceutically acceptable carrier. Such a pharmaceutically acceptable carrier contained in a medicament of the present invention includes any known substances.

Examples of a pharmaceutical acceptable carrier or a suitable formulation material include, but are not limited to, antioxidants, preservatives, colorants, flavoring agents, diluents, emulsifiers, suspending agents, solvents, fillers, bulky agents, buffers, delivery vehicles, and/or pharmaceutical adjuvants. Representatively, a medicament of the present invention is administered in the form of a composition comprising a compound, or a variant or derivative thereof, with at least one physiologically acceptable carrier, excipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are nontoxic to recipients and are preferably inert at the dosages and concentrations employed, and preferably include phosphate, citrate, or other organic acids; ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art. Administration methods may be herein oral, parenteral administration (e.g., intravenous, intramuscular, subcutaneous, intradermal, to mucosa, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing a sugar chain composition having the desired degree of purity with optional physiologically acceptable carriers, excipients, or stabilizers (Japanese Pharmacopeia ver. 14, or a supplement thereto or the latest version; Remington's Pharmaceutical Sciences, 18th Edition, A. R. Gennaro, ed., Mack Publishing Company, 1990; and the like), in the form of lyophilized cake or aqueous solutions.

The amount of the composition of the present invention used in the treatment method of the present invention can be easily determined by those skilled in the art with reference to the purpose of use, a target disease (type, severity, and the like), the patient's age, weight, sex, and case history, the form or type of the cell, and the like. The frequency of the treatment method of the present invention applied to a subject (or patient) is also determined by those skilled in the art with respect to the purpose of use, target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

When the present invention is used for other applications, such as cosmetics, food, agricultural chemicals, and the like, it may be prepared in accordance with limitations defined by the authority.

### Description of Preferred Embodiments

Hereinafter, the present invention will be described by way of embodiments. Embodiments described below are provided only for illustrative purposes. Accordingly, the scope of the present invention is not limited by the embodiments except as by the appended claims.

In one aspect, the present invention provides a composition for increasing the efficiency of introducing a target substance into a cell. The composition of the present invention comprises (a) an actin acting substance. The above-described object of the present invention was achieved by unexpectedly finding that the introduction of a substance (e.g., DNA, RNA, polypeptides, sugar chains or a composite substance thereof, etc.), which is not substantially introduced under normal conditions, is promoted by the action of an actin acting substance (representatively, an extracellular matrix protein). Particularly, it was found that such an actin acting substance has a significant effect of promoting introduction efficiency in genetic manipulation using DNA, such as transfection. Such a finding has not been conventionally known or expected. Attention should be focused onto the present invention which will be a significant breakthrough in gene research.

In a preferred embodiment, an actin acting substance used in the composition of the present invention may be an extracellular matrix protein or a variant or fragment thereof. In the present invention, it was found that an extracellular matrix protein or a variant or fragment thereof unexpectedly acts on actin. Therefore, attention should be focused onto an effect of increasing the efficiency of introducing a substance into cells due to an extracellular matrix protein according to the present invention.

Therefore, in another aspect, the present invention provides a composition for increasing the efficiency of introducing a target substance into a cell, which comprises an extracellular matrix protein or a variant or fragment thereof.

Examples of preferable actin acting substances contained in the composition of the present invention include, but are not limited to, fibronectin, pronectin F, pronectin L, pronectin Plus, laminin, vitronectin, or a variant or fragment thereof.

In a preferred embodiment, an actin acting substance contained in the composition of the present invention, comprises:

(a-1) a protein molecule having at least a Fn1 domain, or a variant thereof;

(a-2) a protein molecule having an amino acid sequence set forth in SEQ ID NO.: 2, 4, 6, 8, 10 or 11, or a variant or fragment thereof;

(b) a polypeptide having the amino acid sequence set forth in SEQ ID NO.: 2, 4, 6, 8, 10 or 11 having at least one mutation selected from the group consisting of at least one amino acid substitution, addition, and deletion, and having a biological activity;

(c) a polypeptide encoded by a splice or alleic mutant of a base sequence set forth in SEQ ID NO.: 1, 3, 5, 7 or 9;

(d) a polypeptide which is a species homolog of the amino acid sequence set forth in SEQ ID NO.: 2, 4, 6, 8, or 11; or

(e) a polypeptide having an amino acid sequence having at least 70% identity to any one of the polypeptides (a-1) to (d), and having a biological activity.

In a preferred embodiment, the number of substitutions, additions, and deletions in (b) is preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. In a certain particular embodiment, the number of substitutions, additions, and deletions may be one or several. A smaller number of substitutions, additions, and deletions are preferable. However, a larger number of substitutions, additions, and deletions are possible as long as a biological activity is retained (preferably, an activity which is similar to or the same as that of an actin acting substance).

In another preferred embodiment, the above-described alleic mutant may preferably have at least 90% homology to the nucleic acid sequence set forth in SEQ ID NO.: 1, 3, 5, 7 or 9. In the same line or the like, for example, such an alleic mutant may preferably have at least 99% homology. In another preferred embodiment, the alleic mutant of (c) may preferably have at least about 90% homology to the amino acid sequence set forth in SEQ ID NO.: 2, 4, 6, 8, 10 or 11. Preferably, the alleic mutant of (c) may have at least about 99% homology to the amino acid sequence set forth in SEQ ID NO.: 2, 4, 6, 8, 10 or 11.

When a gene sequence database is available for the above-described species homolog, the species homolog can be identified by searching the database using the whole or apart of the gene sequence of the extracellular matrix protein of the present invention (e.g., fibronectin, vitronectin, laminin, etc.) as a query sequence. Alternatively, the species homolog can be identified by screening gene libraries of the species using the whole or apart of the gene of the extracellular matrix protein of the present invention (e.g., fibronectin, vitronectin, laminin, etc.) as a probe or a primer. Such identifying methods are well known in the art and described in documents mentioned herein. The species homolog may preferably have at least about 30% homology to the nucleic acid sequence set forth in SEQ ID NO.: 1, 3, 5, 7 or 9, for example. The species homolog may preferably have at least about 50% homology to the nucleic acid sequence set forth in SEQ ID NO.: 1, 3, 5, 7 or 9. In another preferred embodiment, the species homolog may preferably have at least about 30% homology to the amino acid sequence set forth in SEQ ID NO.: 2, 4, 6, 8, 10 or 11. The species homolog may preferably have at least about 50% homology to the amino acid sequence set forth in SEQ ID NO.: 2, 4, 6, 8, 10 or 11.

In a preferred embodiment, the identity to any one of the polypeptides (a-1) to (d) may be at least about 80%, more preferably at least about 90%, even more preferably at least about 98%, and most preferably at least about 99%.

In a more preferred embodiment, the nucleic acid sequence or amino acid sequence may be a sequence related to SEQ ID NO.: 1, 2 or 11 (fibronectin sequence). Therefore, the description “homology thereof” may be replaced with SEQ ID NO.: 1, 2 or 11 in a more preferred embodiment.

In one embodiment, the actin acting substance of the present invention may comprise a Fn1 domain of amino acids 21 to 577 of SEQ ID NO.: 11.

In another preferred embodiment, the actin acting substance may be fibronectin or a variant or fragment thereof, and more preferably fibronectin.

The concentration of the actin acting substance can be easily determined by those skilled in the art with reference to the present specification. For example, such a concentration may be at least about 0.1 μg/μL, preferably about 0.2 μg/μL, and more preferably 0.5 μg/μL. In one embodiment, the introduction efficiency reaches a plateau in the case of a concentration of about 0.5 μg/μLl or more. A preferable concentration range may be from about 0.5 μg/μL to 2.0 μg/μL.

In another aspect, the present invention relates to a composition for increasing the efficiency of introducing a target substance into a cell, wherein the composition comprises an adhesion agent. Fibronectin has been known as an adhesion agent. However, it was not known that such an adhesion agent can be used to increase the efficiency of introducing a target substance into a cell (e.g., transfection, etc.). Therefore, the present invention can be considered to be attributed to the unexpected effect of adhesion agents. Such adhesion agents are described in detail above. Therefore, in the following various embodiments, such adhesion agents can be used instead of actin acting substances.

In an embodiment in which gene introduction is intended, the composition of the present invention may preferably comprise a gene introduction reagent. This is because such a gene introduction reagent synergistically exhibits the effect of increasing the efficiency of introduction of the present invention.

In a preferred embodiment, such a gene introduction reagent includes, but is not limited to, at least one substance selected from the group consisting of cationic polymers, cationic lipids, and calcium phosphate. More preferably, examples of gene introduction reagents include, but are not limited to, Effectene, TransFast™, Tfx™-20, SuperFect, PolyFect, LipofectAMINE 2000, JetPEI, ExGen 500, and the like.

In another embodiment, the composition of the present invention may further comprise a particle. This is because use of such a particle can lead to an increase in the efficiency of introducing a substance into a cell, particularly a target cell. Preferable examples of such a particle include, but are not limited to, metal colloids, such as gold colloid, and the like.

In another preferred embodiment, the composition of present invention may further comprise a salt. Though not wishing to be bound by any theory, use of such a salt enhances the fixing effect when a solid phase support is used. Alternatively, it is considered that the three-dimensional structure of a target substance can be retained in a more appropriate form.

Any inorganic or organic salt may be used as the above-described salt. Use of a mixture of a plurality of salts is more preferable than use of a single salt. Examples of such a mixture of a plurality of salts include, but are not limited to, salts contained in buffers, salts contained in media, and the like.

In another aspect, the present invention provides a kit for increasing the efficiency of introducing a gene. The kit comprises: (a) a composition comprising an actin acting substance; and (b) a gene introduction reagent. Such an actin acting substance may be selected and used as described in detail above for the composition of the present invention for increasing the efficiency of introducing a target substance into a cell. An appropriate form of the actin acting substance can be selected by those skilled in the art based on the present specification. When the present invention is provided in the form of such a kit, the kit may comprise instructions. The instructions may be prepared in accordance with a format defined by an authority of a country in which the present invention is practiced, explicitly describing that the instructions are approved by the authority. The present invention is not limited to this. The instructions are typically provided in the form of a manual and in paper media. The instructions are not so limited and may be provided in the form of electronic media (e.g., web sites, electronic mails, and the like provided on the Internet). Such an actin acting substance may be selected and used as described in detail above for the composition of the present invention for increasing the efficiency of introducing a target substance into a cell. Therefore, preferably, the actin acting substance may be an extracellular matrix protein (e.g., fibronectin, vitronectin, laminin, etc.) or a variant thereof. More preferably, fibronectin or a variant or fragment thereof may be used.

In another aspect, the present invention provides a composition for introducing a target substance into a cell. The present invention was completed by unexpectedly finding that the introduction of a substance (e.g., DNA, RNA, polypeptides, sugar chains or a composite substance thereof, etc.), which is not substantially introduced under normal conditions, is promoted by the action of an actin acting substance (representatively, an extracellular matrix protein). In this case, the present invention is provided in the form of a composition comprising a target substance and an actin acting substance. Such an actin acting substance may be selected and used as described in detail above for the composition of the present invention for increasing the efficiency of introducing a target substance into a cell. Therefore, preferably, the actin acting substance may be an extracellular matrix protein (e.g., fibronectin, vitronectin, laminin, etc.) or a variant thereof. More preferably, fibronectin or a variant or fragment thereof may be used.

Examples of a target substance contained in the composition of the present invention for introducing the target substance into a cell include, but are not limited to, DNA, RNA, polypeptides, sugars, and complexes thereof, and the like. In a particular preferred embodiment, DNA may be selected as a target substance. Such DNA may preferably encode a gene of interest when gene expression is intended. Therefore, in an embodiment in which transfection is intended, a target substance may include DNA encoding a gene sequence to be transfected. In another preferred embodiment, RNA is selected as a target substance. Such RNA may preferably encode a gene of interest when gene expression is intended. In this case, RNA encoding a gene sequence may be preferably used along with a gene introduction agent suitable for RNA.

In an embodiment in which gene introduction is intended, the composition of the present invention for introducing a target substance into a cell may further comprise a gene introduction reagent. Though not wishing to be bound by any theory, in one embodiment, it is considered that such a gene introduction reagent and an actin acting substance found in the present invention function in cooperation with each other, thereby achieving a higher efficiency of introducing a gene into a cell than that of conventional techniques.

In a preferred embodiment, examples of such a gene introduction reagent contained in the composition of the present invention include, but are not limited to, cationic polymers, cationic lipids, polyamine-based reagents, polyimine-based reagents, calcium phosphate, and the like.

In a preferred embodiment, the composition of the present invention, for introducing a target substance into a cell may be a liquid phase. In the case of a liquid phase, the present invention is useful as, for example, a liquid phase transfection system.

In another preferred embodiment, the composition of the present invention for introducing a target substance into a cell may be a solid phase. In the case of a solid phase, the present invention is useful as, for example, a solid phase transfection system. Preferable examples of such a solid phase transfection system include, but are not limited to, microtiter plate-based transfection systems, array (or chip)-based transfection systems, and the like. For the introduction of a polypeptide, either a liquid phase or a solid phase may be useful.

In another aspect, the present invention provides a device for introducing a target substance into a cell. In the device, a composition comprising A) the target substance and B) an actin acting substance is fixed onto a solid phase support. The device of the present invention was completed by unexpectedly finding that the introduction of a substance (e.g., DNA, RNA, polypeptides, sugar chains or a composite substance thereof, etc.), which is not substantially introduced under normal conditions, is promoted by the action of an actin acting substance (representatively, an extracellular matrix protein). In this case, a composition comprising a target substance and an actin acting substance is fixed onto a solid phase support. Such an actin acting substance may be selected and used as described in detail above for the composition of the present invention for increasing the efficiency of introducing a target substance into a cell. Therefore, preferably, the actin acting substance may be an extracellular matrix protein (e.g., fibronectin, vitronectin, laminin, etc.) or a variant thereof. More preferably, fibronectin or a variant or fragment thereof may be used.

Examples of a target substance contained in the device of the present invention for introducing the target substance into a cell include, but are not limited to, DNA, RNA, polypeptides, sugars, and complexes thereof, and the like. In a particular preferred embodiment, DNA may be selected as a target substance. Such DNA may preferably encode a gene of interest when gene expression is intended. Therefore, in an embodiment in which transfection is intended, a target substance may include DNA encoding a gene sequence to be transfected.

In an embodiment in which gene introduction is intended, the device of the present invention may further comprise a gene introduction reagent. Though not wishing to be bound by any theory, in one embodiment, it is considered that such a gene introduction reagent and an actin acting substance found in the present invention function in cooperation with each other, thereby achieving a higher efficiency of introducing a gene into a cell than that of conventional techniques.

In a preferred embodiment, a solid phase support used in the device of the present invention may be selected from the group consisting of plates, microwell plates, chips, slide glasses, films, beads, and metals.

In a particular embodiment, when the device of the present invention uses a chip as a solid phase support, the device may be called an array. In such an array, biological molecules (e.g., DNA, proteins, etc.) to be introduced are typically arranged or patterned on a substrate. Such an array used for transfection is also herein called a transfection array. In the present invention, it was revealed that transfection takes place for stem cells, which cannot be achieved by conventional systems. Therefore, the composition, device and method of the present invention which use an actin acting substance can be used to provide a transfection array capable of transfection of any cell. This is an unexpected effect which cannot be conventionally achieved.

A solid phase support used in the device of the present invention may be preferably coated. Coating improves the quality of a solid phase support and substrate (e.g., elongation of life span, improvement in resistance to hostile environment, such as resistance to acids, etc.), affinity to a substance integrated with a solid phase support or substrate, and the like. In a preferred embodiment, such coating is obtained with a coating agent, such as poly-L-lysine, silane (e.g., APS (γ-aminopropyl silane)), MAS, hydrophobic fluorine resin, silane (e.g., epoxy silane or mercaptosilane), a metal (e.g., gold, etc.), or the like. Preferably, a coating agent may be poly-L-lysine.

In another aspect, the present invention provides a method for increasing the efficiency of introducing a target substance into a cell. The present invention represents a first discovery and was completed by unexpectedly finding that the introduction of a substance (e.g., DNA, RNA, polypeptides, sugar chains or a composite substance thereof, etc.), which is not substantially introduced under normal conditions, is efficiently introduced into cells by presenting (preferably contacting) the target substance along with an actin acting substance to the cells. The method of the present invention comprises: A) providing the target substance; B) providing an actin acting substance; and further C) contacting the target substance and the actin acting substance to the cell. The target substance and the actin acting substance may be provided together or separately. Such an actin acting substance may be selected and used as described in detail above for the composition of the present invention for increasing the efficiency of introducing a target substance into a cell. Such selection may be made as appropriate by those skilled in the art based on the present specification. Therefore, preferably, the actin acting substance may be an extracellular matrix protein (e.g., fibronectin, vitronectin, laminin, etc.) or a variant thereof. More preferably, fibronectin or a variant or fragment thereof may be used.

Examples of a target substance contained in the method of the present invention include, but are not limited to, DNA, RNA, polypeptides, sugars, and complexes thereof, and the like. In a particular preferred embodiment, DNA may be selected as a target substance. Such DNA may preferably encode a gene of interest when gene expression is intended. Therefore, in an embodiment in which transfection is intended, a target substance may include DNA encoding a gene sequence to be transfected.

In an embodiment in which gene introduction is intended, the method of the present invention may further comprise a gene introduction reagent. Though not wishing to be bound by any theory, in one embodiment, it is considered that such a gene introduction reagent and an actin acting substance found in the present invention function in cooperation with each other, thereby achieving a higher efficiency of introducing a gene into a cell than that of conventional techniques. The gene introduction reagent and the target substance and/or the actin acting substance may be provided together or separately. Preferably, the target substance and the gene introduction reagent may be advantageously formed into a complex before providing the actin acting substance. Though not wishing to be bound by any theory, it is considered that introduction efficiency is increased by providing the target substance and the like in such an order.

In a preferred embodiment, examples of such a gene introduction reagent used in the method of the present invention include, but are not limited to, cationic polymers, cationic lipids, polyamine-based reagents, polyimine-based reagents, calcium phosphate, and the like.

Any cell can be targeted in the present invention as long as the introduction of a target substance is intended. Examples of cells include, but are not limited to, stem cells, somatic cells, and the like. The present invention has a significant effect that a target substance can be introduced (e.g., transfected, etc.) into substantially all types of cells (e.g., stem cells, somatic cells, etc.). This effect can be said to be an unexpected effect which is not possessed by conventional methods. Preferably, target stem cells may include, without limitation, tissue stem cells and also embryonic stem cells. Though not wishing to be bound by any theory, among stem cells, it is considered that tissue stem cells have higher introduction efficiency than that of embryonic stem cells.

In a particular embodiment, a part or the whole of the method of the present invention for introducing a target substance into a cell may be performed in a liquid phase. In another particular embodiment, a part or the whole of the method of the present invention for introducing a target substance into a cell may be performed on a solid phase. Therefore, the method of the present invention for introducing a target substance into a cell may be performed using a combination of a liquid phase and a solid phase.

In another aspect, the present invention provides a method for increasing the efficiency of introducing a target substance into a cell using a solid phase support. The present invention represents a first discovery and was completed by unexpectedly finding that the introduction of a substance (e.g., DNA, RNA, polypeptides, sugar chains or a composite substance thereof, etc.), which is not substantially introduced under normal conditions, is efficiently introduced into cells by presenting (preferably contacting) the target substance along with an actin acting substance to the cells. The effect of increasing introduction efficiency of a target substance (particularly DNA, preferably DNA containing a sequence encoding a gene to be transfected) by using a solid phase support cannot be achieved, or at least expected, by conventional techniques. Thus, the present invention is a significant breakthrough in the art. The method of the present invention using a solid phase support comprises: I) fixing a composition comprising A) a target substance and B) an actin acting substance to a solid support; and II) contacting the cell to the composition on the solid support. Such an actin acting substance may be selected and used as described in detail above for the composition of the present invention for increasing the efficiency of introducing a target substance into a cell. Such selection may be made as appropriate by those skilled in the art based on the present specification. Preferably, the actin acting substance may be an extracellular matrix protein (e.g., fibronectin, vitronectin, laminin, etc.) or a variant thereof. More preferably, fibronectin or a variant or fragment thereof may be used.

Naked DNA may be used as a target substance. Preferably, DNA may be advantageously provided along with a control sequence (e.g., a promoter, etc.) using a vector (e.g., a plasmid, etc.). In such a case, preferably, DNA may be operably linked to be the control sequence.

Preferably, the method of the present invention may further comprise providing a gene introduction reagent, wherein the gene introduction reagent is contacted with the cell. Use of a gene introduction reagent is preferable because of a further improvement in introduction efficiency of the method of the present invention. It is well known in the art to provide a gene introduction reagent. For example, without limitation, a solution containing a gene introduction reagent dissolved therein is added to an experimentation system. Preferably, a gene introduction reagent and DNA (a target substance) are formed into a complex before providing an actin acting substance. Though not wishing to be bound by any theory, it was revealed that by providing the target substance and the like in such an order, the efficiency of introducing a target substance into a cell on a solid phase support is dramatically increased.

In one embodiment, the gene introduction reagent (e.g., cationic lipid)-target substance complex comprises a target substance (e.g., DNA in an expression vector) and a gene introduction reagent and is dissolved in an appropriate solvent, such as water or deionized water. The resultant solution is spotted onto a surface of a slide or the like, thereby producing a surface on which the gene introduction reagent-target substance complex is adhered to specific positions. Thereafter, an actin acting substance is added as appropriate. The spots of the gene introduction reagent-target substance complex are adhered to the slide, and are dried well so that the spots will remain adhered to the same position under the subsequent steps in the method. For example, a gene introduction reagent-target substance complex is spotted on a slide (e.g., a glass slide, etc.) or chip coated with poly-L-lysine (available from Sigma, Inc., etc.) manually or using a microarray producing machine. Thereafter, the slide or chip is dried under reduced pressure at room temperature or a temperature higher than room temperature, thereby adhering the DNA spots onto the slide. The time required for drying well depends on several factors, such as the amount of a mixture provided on the surface, the temperature and humidity conditions, and the like. In the present invention, the actin acting substance may be preferably provided after adhesion of the complex.

The concentration of DNA in a mixture may be experimentally determined, but is generally in the range of from about 0.01 μg/μl to about 0.2 μg/μl. In a particular embodiment, the range is from about 0.02 μg/μl to about 0.10 μg/μl. Alternatively, the concentration of DNA in a gene introduction reagent-target substance complex is in the range of from about 0.01 μg/μl to about 0.5 μg/μl, from about 0.01 μg/μl to about 0.4 μg/μl, or from about 0.01 μg/μl to about 0.3 μg/μl. Similarly, the concentration of another carrier polymer, such as an actin acting substance or a gene introduction reagent, may be experimentally determined for each application, but are generally in the range of from 0.01% to 0.5%. In a particular embodiment, the range is from about 0.05% to about 0.5%, from about 0.05% to about 0.2%, or from about 0.1% to about 0.2%. The final concentration of DNA (e.g., DNA in an actin acting substance) in an actin acting substance-target substance is generally in the range of from about 0.02 μg/μl to about 0.1 μg/μl. In another embodiment, DNA may have a final concentration of about 0.05 μg/μl.

DNA used in the present invention may be provided in a vector of any type, such as a plasmid or a virus. A vector containing DNA of interest may be introduced into a cell, and thereafter, DNA may be expressed in the cell. For example, a CMV driven expression vector may be used. Commercially available plasmid vectors (e.g., pEGFP (Clontech) or pcDNA 3 (Invitrogen), etc.) or viral vectors may be used. In this embodiment, after the spots containing the gene introduction reagent-target substance complex is dried, the surface having the spots is coated with a transfection reagent based on an appropriate amount of lipid. The resultant product is maintained (incubated) under conditions suited for the formation of a complex of the DNA and the gene introduction reagent (e.g., a transfection reagent, such as a cationic lipid, etc.) in the spot. Preferably, an actin acting substance may be provided subsequently or simultaneously. In one embodiment, the resultant product is incubated at 25° C. for about 20 minutes. Thereafter, the gene introduction reagent is removed. Thus, the surface having DNA (DNA in a complex of the DNA and the transfection reagent) is produced. Cells in appropriate culture medium are plated on the surface. The resultant product (the surface having the DNA and the plated cells) is maintained under conditions which allow the DNA to enter the plated cells.

In the present invention, a time of about 1 to 2 cell cycles is sufficient for transfection. The time required for transfection varies depending on the cell type and conditions. The time appropriate for a specific combination may be experimentally determined by those skilled in the art. After a sufficient time has passed, transfection efficiency, expression of encoded products, an influence on cells, and the like can be evaluated using known methods. For example, these parameters can be determined by detection of immunofluorescence, or enzymatic immunological cytology, in situ hybridization, autoradiography, or other means for detecting an influence of DNA expression or DNA products or DNA itself on cells having the introduced DNA. When immunofluorescence is used for detection of expression of a protein encoded by DNA, an antibody which binds to a protein and is tagged with a fluorescent label (e.g., an antibody is applied to a slide under appropriate conditions which allow the antibody to bind to a protein) is used and a position (a spot or region on a surface) containing a protein is identified by detecting fluorescence. The presence of fluorescence indicates that transfection occurs at a position from which the fluorescence is emitted, i.e., the encoded protein is expressed. The presence of a signal detected on the slide by the above-described method indicates that transfection and expression of a coded product or introduction of DNA into the cell occur at a position from which the signal is detected. The identity of DNA provided at specific positions may be either known or unknown. Therefore, when expression occurs, the identity of an expressed protein may be either known or unknown. Such information may be preferably known. This is because such information can be correlated with conventional information.

All patents, published patent applications and publications cited herein are incorporated by reference as if set forth fully herein.

The preferred embodiments of the present invention have been heretofore described for a better understanding of the present invention. Hereinafter, the present invention will be described by way of examples. Examples described below are provided only for illustrative purposes. Accordingly, the scope of the present invention is not limited by the embodiments and examples specified herein except as by the appended claims.

### EXAMPLES

Hereinafter, the present invention will be described in greater detail by way of examples, though the present invention is not limited to the examples below. Reagents, supports, and the like were commercially available from Sigma (St. Louis, USA), Wako Pure Chemical Industries (Osaka, Japan), Matsunami Glass (Kishiwada, Japan) unless otherwise specified.

### Example 1

### Preparation of Actin Acting Substance Mixture

Formulations below were prepared in Example 1.

As candidates for an actin acting substance, various extracellular matrix proteins and variants or fragments thereof were prepared in Example 1 as listed below. Fibronectin and the like were commercially available. Fragments and variants were obtained by genetic engineering techniques:

1) fibronectin (SEQ ID NO.: 11);
2) fibronectin 29 kDa fragment;
3) fibronectin 43 kDa fragment;
4) fibronectin 72 kDa fragment;
5) fibronectin variant (SEQ ID NO.: 11, alanine at 152 was substituted with leucine);
6) pronectin F (Sanyo Chemical Industries, Kyoto, Japan);
7) pronectin L (Sanyo Chemical Industries);
8) pronectin Plus (Sanyo Chemical Industries);
9) laminin (SEQ ID NO.: 6);
10) RGD peptide (tripeptide);
11) RGD-containing 30-kDa peptide;
12) 5 amino acids of laminin (SEQ ID NO.: 17); and
13) gelatin.

Plasmids were prepared as DNA for transfection. Plasmids, pEGFP-N1 and pDsRed2-N1 (both from BD Biosciences, Clontech, CA, USA) were used. In these plasmids, gene expression was under the control of cytomegalovirus (CMV). The plasmid DNA was amplified in E. coli (XL1 blue, Stratgene, TX, USA) and the amplified plasmid DNA was used as a complex partner. The DNA was dissolved in distilled water free from DNase and RNase.

The following transfection reagents were used: Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France), and ExGen 500 (R0511, Fermentas Inc., MD). These transfection reagents were added to the above-described DNA and actin acting substance in advance or complexes thereof with the DNA were produced in advance.

The thus-obtained solution was used in assays using transfection arrays described below.

### Example 2

### Improvement in Transfection Efficiency in Liquid Phase

In Example 2, an improvement in the transfection efficiency of solid phase was observed. The protocol used in Example 2 will be described below.

The protocol for liquid phase transfection is in accordance with instructions provided along with each of Effectene, LipofectAMINE 2000, JetPEI, or TransFast.

In Example 2, effects of the above-prepared actin acting substances were studied in the presence or absence thereof in liquid phase transfection.

An actin acting substance was preserved as a stock having a concentration of 10 μg/μL in ddH₂O. All dilutions were made using PBS, ddH₂O, or Dulbecco's MEM. A series of dilutions, for example, 0.2 μg/μL, 0.2 μg/μL, 0.4 μg/μL, 0.53 μg/μL, 0.6 μg/μL, 0.8 μg/μL, 1.0 μg/μL, 1.07 μg/μL, 1.33 μg/μL, and the like, were formulated.

As a result, it was revealed that these actin acting substances increased the efficiency of liquid phase transfection. Particularly, it was revealed that fibronectin had a significant effect of increasing the efficiency.

### Example 3

### Improvement in Transfection Efficiency in Solid Phase

In Example 3, an improvement in the transfection efficiency of solid phase was observed. The protocol used in Example 3 will be described below.

(Protocol)

The final concentration of DNA was adjusted to 1 μg/μL. An actin acting substance was preserved as a stock having a concentration of 10 μg/μL in ddH₂O. All dilutions were made using PBS, ddH₂O, or Dulbecco's MEM. A series of dilutions, for example, 0.2 μg/μL, 0.27 μg/μL, 0.4 μg/μL, 0.53 μg/μL, 0.6 μg/μL, 0.8 μg/μL, 1.0 μg/μL, 1.07 μg/μL, 1.33 μg/μL, and the like, were formulated.

Transfection reagents were used in accordance with instructions provided by each manufacturer:

Plasmid DNA was removed from a glycerol stock and amplified in 100 mL L-amp overnight. Qiaprep Miniprep or Qiagen Plasmid Purification Maxi was used to purify DNA in accordance with a standard protocol provided by the manufacturer.

In Example 3, the following 5 cells were used to confirm an effect: human mesenchymal stem cell (hMSCs, PT-2501, Cambrex BioScience Walkersville, Inc., MD); human embryonic renal cell (HEK293, RCB1637, RIKENCellBank, JPN); NIH3T3-3 cell (RCB0150, RIKEN Cell Bank, JPN); HeLa cell (RCB0007, RIKEN Cell Bank, JPN); and HepG2 (RCB1648, RIKEN Cell Bank, JPN). These cells were cultured in DMEM/10% IFS containing L-glut and pen/strep.

(Dilution and DNA Spots)

Transfection reagents and DNA were mixed to form a DNA-transfection reagent complex. The complex formation requires a certain period of time. Therefore, the mixture was spotted onto a solid phase support (e.g., a poly-L-lysine slide) using an arrayer. In Example 3, as a solid phase support, an APS slide, a MAS slide, and a uncoated slide were used as well as a poly-L-lysine slide. These slides are available from Matsunami Glass (Kishiwada, Japan) or the like.

For complex formation and spot fixation, the slides were dried overnight in a vacuum dryer. Drying was performed in the range of 2 hours to 1 week.

Although the actin acting substance might be used during the complex formation, it was also used immediately before spotting in Example 3.

(Formulation of Mixed Solution and Application to Solid Phase Supports)

300 μL of DNA concentrated buffer (EC buffer)+16 μL of an enhancer were mixed in an Eppendorf tube. The mixture was mixed with a Vortex, followed by incubation for 5 minutes. 50 μL of a transfection reagent (Effectene, etc.) was added to the mixture, followed by mixing by pipetting. To apply a transfection reagent, an annular wax barrier was formed around the spots on the slide. 366 μL of the mixture was added to the spot region surrounded by the wax, followed by incubation at room temperature for 10 to 20 minutes. Thereby, the fixation to the support was manually achieved.

(Distribution of Cells)

Next, a protocol for adding cells will be described. Cells were distributed for transfection. The distribution was typically performed by reduced-pressure suction in a hood. A slide was placed on a dish, and a solution containing cells was added to the dish for transfection. The cells were distributed as follows.

The growing cells were distributed to a concentration of 10⁷ cells/25 mL. The cells were plated on the slide in a 100×100×15 mm squared Petri dish or a 100 mm (radius))×15 mm circular dish. Transfection was conducted for about 40 hours. This period of time corresponded to about 2 cell cycles. The slide was treated for immunofluorescence.

(Evaluation of Gene Introduction)

Gene introduction was evaluated by detection using, for example, immunofluorescence, fluorescence microscope examination, laser scanning, radioactive labels, and sensitive films, or emulsion.

When an expressed protein to be visualized is a fluorescent protein, such a protein can be observed with a fluorescence microscope and a photograph thereof can be taken. For large-sized expression arrays, slides may be scanned using a laser scanner for storage of data. If an expressed protein can be detected using fluorescence antibodies, an immunofluorescence protocol can be successively performed. If detection is based on radioactivity, the slide may be adhered as described above, and autoradiography using film or emulsion can be performed to detect radioactivity.

(Laser Scanning and Quantification of Fluorescence Intensity)

To quantify transfection efficiency, the present inventors use a DNA microarray scanner (GeneTAC UC4×4, Genomic Solutions Inc., MI). Total fluorescence intensity (arbitrary unit) was measured, and thereafter, fluorescence intensity per unit surface area was calculated.

(Cross-Sectional Observation by Confocal Scanning Microscope)

Cells were seeded on tissue culture dishes at a final concentration of 1×10⁵ cells/well and cultured in appropriate medium (Human Mesenchymal Cell Basal Medium (MSCGM BulletKit PT-3001, Cambrex BioScience Walkersville, Inc., MD). After fixation of the cell layer with 4% paraformaldehyde solution, SYTO and Texas Red-X phalloidin (Molecular Probes Inc., OR, USA) was added to the cell layer for observation of nuclei and F-actin. The samples emitting light due to gene products and the stained samples were observed with a confocal laser microscope (LSM510: Carl Zeiss Co., Ltd., pin hole size=Ch1=123 Ch2=108 μm, image interval=0.4) to obtain cross sectional views.

(Results)

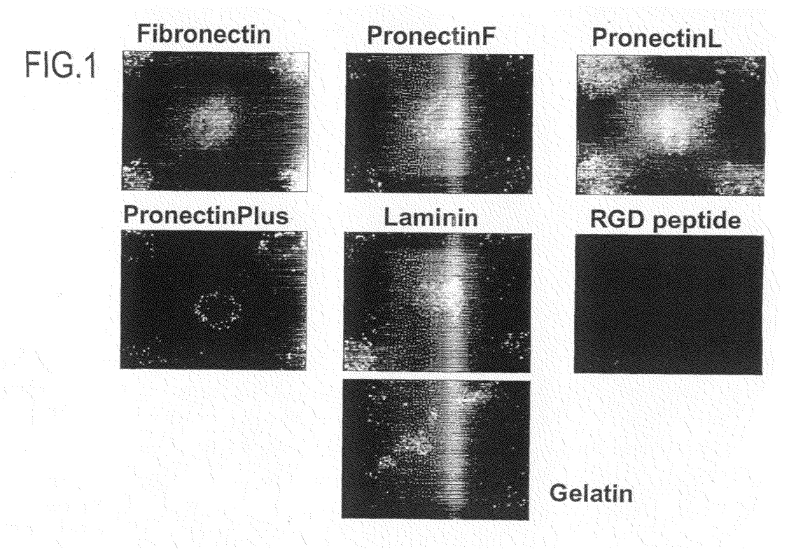

FIG. 1 shows the results of experiments in which various actin acting substances and HEK293 cells were used where gelatin was used as a control.

As can be seen from the results, whereas transfection was not very successful in a system using gelatin, transfection took place to a significant level in systems using fibronectin, pronectin (pronectin F, pronectin L, pronectin Plus) which is a variant of fibronectin, and laminin. Therefore, it was demonstrated that these molecules significantly increased transfection efficiency. Use of the RGD peptide alone exhibited substantially no effect.

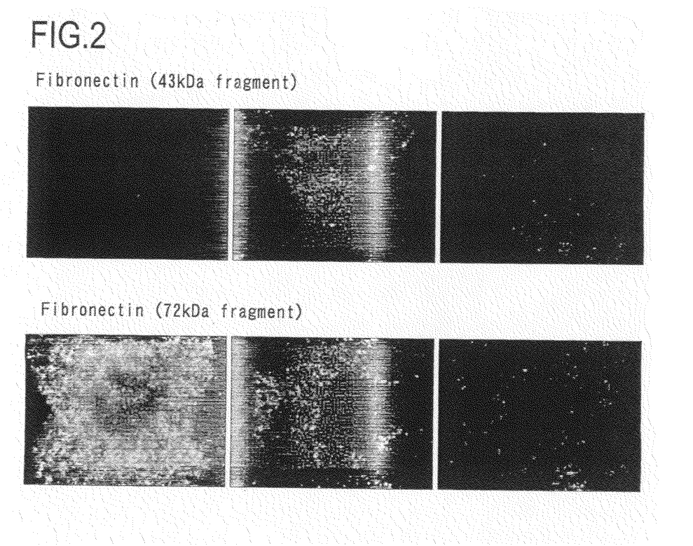

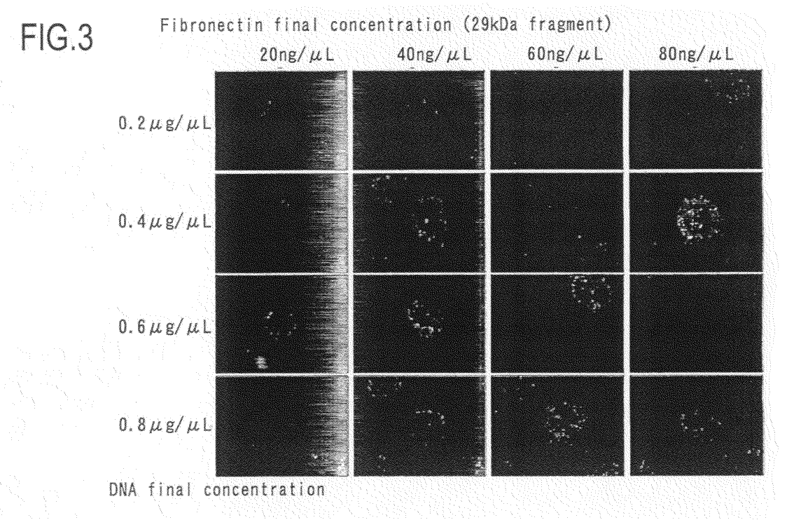

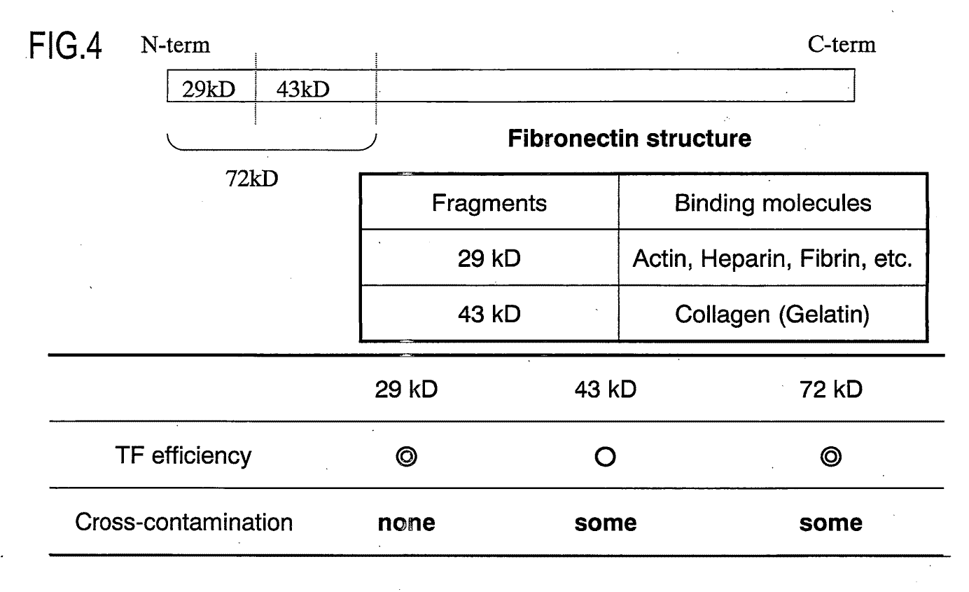

FIGS. 2 and 3 show transfection efficiency when fibronectin fragments were used. FIG. 4 shows the summary of the results. 29 kDa and 72 kDa fragments exhibited a significant level of transfection activity, while a 43 kDa fragment had activity but its level was low. Therefore, it was suggested that an amino acid sequence contained in the 29 kDa fragment played a role in an increase in transfection efficiency. Substantially no contamination was found in the case of the 29 kDa fragment, while contamination was observed in the case of the other two fragments (43 kDa and 72 kDa). Therefore, only the 29 kDa domain may be preferably used as an actin acting substance. When only the RGD peptide was used, the activity to increase transfection efficiency was not exhibited. The 29-kDa peptide exhibited activity. Such a system with additional 6 amino acids of laminin (higher molecular weight) exhibited transfection activity. Therefore, these peptide sequences may also play an important role in the activity to increase transfection efficiency, without limitation. In such a case, a molecular weight of at least 5 kDa, preferably at least 10 kDa, and more preferably at least 15 kDa may be required for an increase in transfection efficiency.

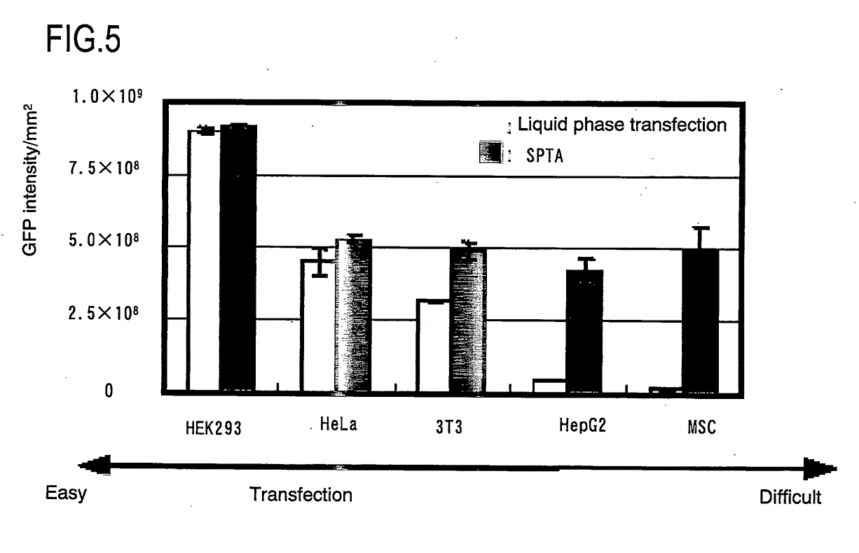

Next, FIG. 5 shows the result of studies on transfection efficiency of cells. In FIG. 5, HEK293 cells, HeLa cells, and 3T3 cells, which were conventionally transfectable, and HepG2 cells and mesenchymal stem cells (MSC) which were conventionally believed to be substantially impossible to transfect, were used to show an effect of the transfection method of the present invention. The vertical axis represents the intensity of GFP.

In FIG. 5, the transfection method of the present invention using a solid phase support was compared with a conventional liquid phase transfection method. The conventional liquid phase transfection method was conducted in accordance with a protocol recommended by the kit manufacturer.

As can be seen from FIG. 5, transfection efficiency comparable to HeLa and 3T3 was achieved in HepG2 cells and mesenchymal stem cells (MSC) which were conventionally believed to be substantially impossible to transfect, as well as HEK293 cells, HeLa cells, and 3T3 cells, which were conventionally transfectable. Such an effect was not achieved by conventional transfection systems. The present invention was the first to provide a system which can increase transfection efficiency for substantially all cells and can provide practicable transfection to all cells. By using solid phase conditions, cross contamination was significantly reduced. Therefore, it was demonstrated that the present invention using a solid phase support is appropriate for production of an integrated bioarray.

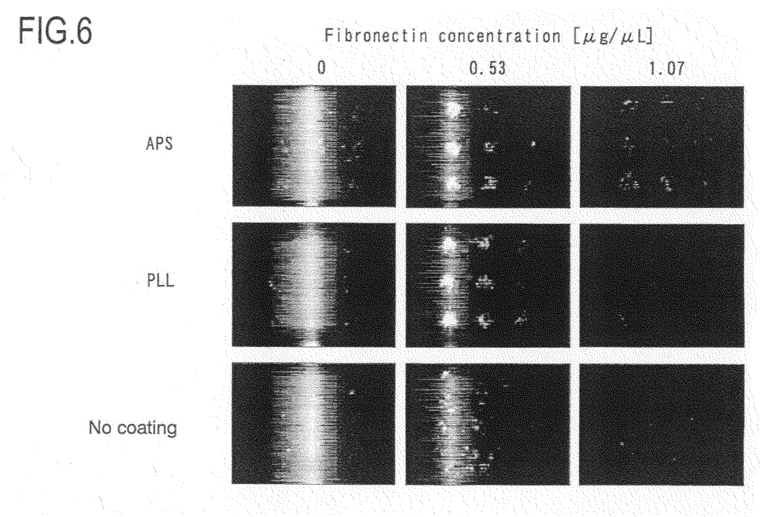

Next, FIG. 6 shows the results of transfection when various plates were used. As can be seen from the results of FIG. 6, when coating was provided, contamination was reduced as compared with when coating was not provided and transfection efficiency was increased.

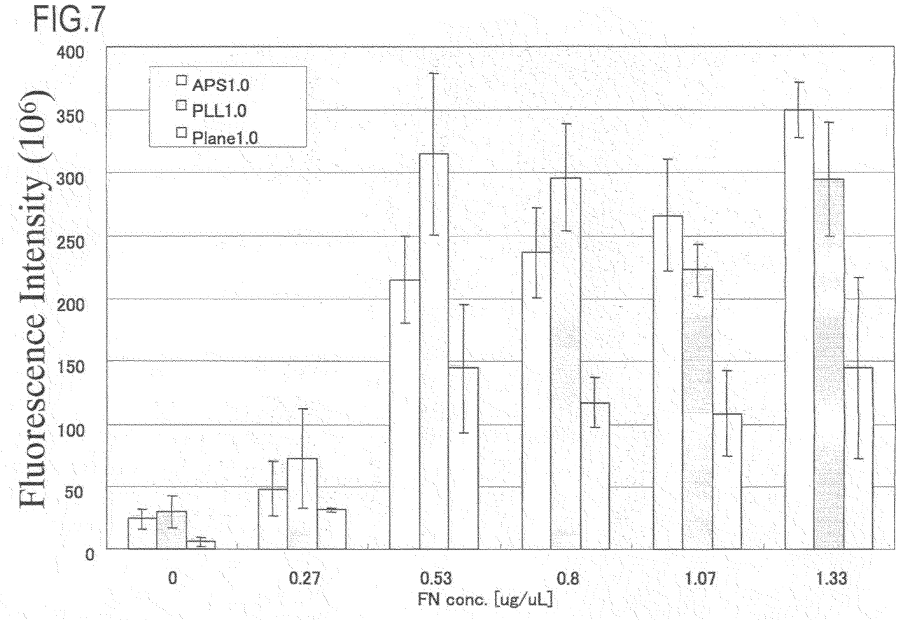

Next, FIG. 3 shows the results of transfection where the concentration of fibronectin was 0, 0.27, 0.53, 0.8, 1.07, and 1.33 (μg/μL, for each). In FIG. 7, slides coated with PLL (poly-L-lysine) and APS and uncoated slides were shown.

As can be seen from the results of FIG. 7, transfection efficiency was increased with an increase in fibronectin concentration. Note that in the case of PLL coating and the absence of coating, the transfection efficiency reached a plateau at a fibronectin concentration of more than 0.53 μg/μL. In the case of APS, it was found that the effect was further increased at a fibronectin concentration of more than of 1.07 μg/μL.

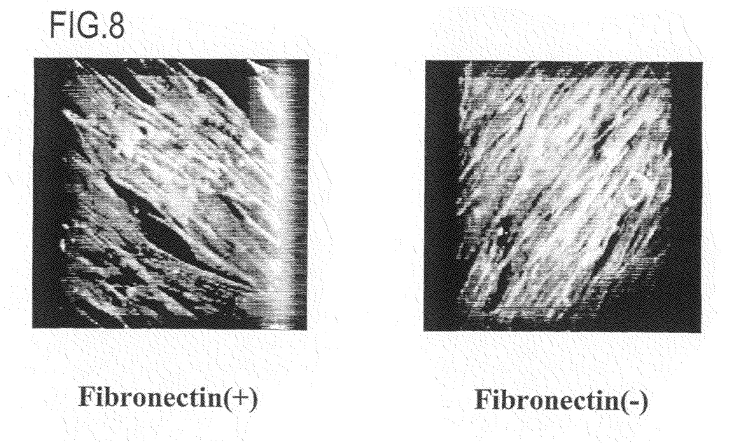

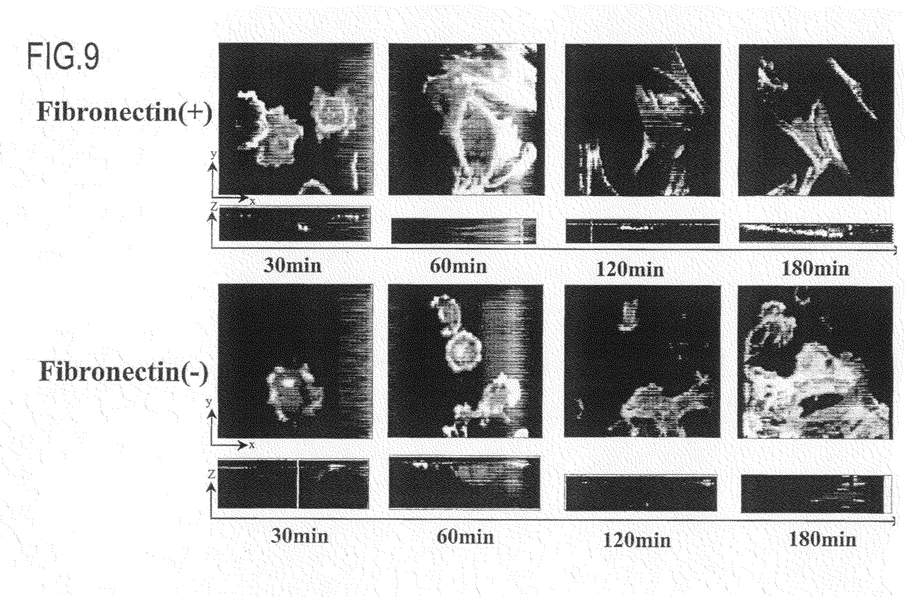

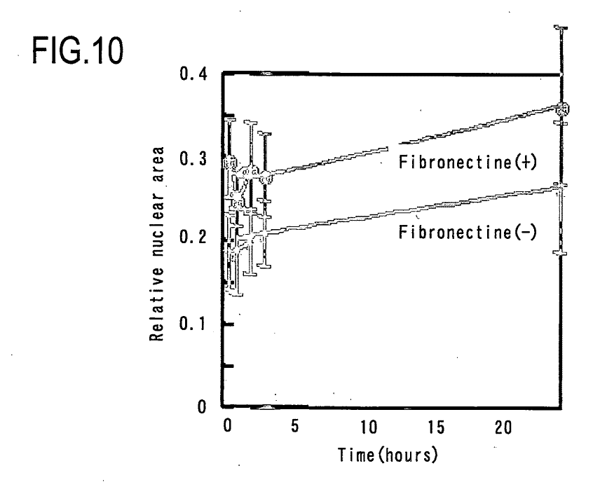

Next, FIG. 8 shows photographs indicating cell adhesion profiles in the presence or absence of fibronectin. FIG. 9 shows cross-sectional photographs. It was revealed that the shapes of adherent cells were significantly different (FIG. 8). The full extension of cells was found for the initial 3 hours of culture in the presence of fibronectin, while extension was limited in the absence of fibronectin (FIG. 9). Considering the behavior of filaments (FIG. 9) and the results of the time-lapse observation, it was considered that an actin acting substance, such as fibronectin, attached to a solid phase support had an influence on the shape and orientation of actin filaments, and the efficiency of introduction of a substance into a cell, such as transfection efficiency or the like, is increased. Specifically, actin filaments quickly change their location in the presence of fibronectin, and disappear from the cytoplasmic space under the nucleus as the cell extends. It is considered that actin depletion in the perinuclear space, which is induced by an actin acting substance, such as fibronectin, allows the transport of a target substance, such as DNA or the like, into cells or nuclei. Though not wishing to be bound by any theory, the reason is considered to be that the viscosity of cytoplasm is reduced and positively charged DNA particles are prevented from being trapped by negatively charged actin filaments. Additionally, it is considered that the surface area of the nucleus is significantly increased in the presence of fibronectin (FIG. 10), possibly facilitating the transfer of a target substance, such as DNA or the like, into nuclei.

### Example 4

### Application to Bioarrays

Next, larger-scale experiments were conducted to determine whether or not the above-described effect was demonstrated when arrays were used.

(Experimental Protocols)

(Cell Sources, Culture Media, and Culture Conditions)

In this example, five different cell lines were used: human mesenchymal stem cells (hMSCs, PT-2501, Cambrex BioScience Walkersville, Inc., MD), human embryonic kidney cell HEK293 (RCB1:637, RIKEN Cell Bank, JPN), NIH3T3-3 (RCB0150, RIKEN Cell Bank, JPN), HeLa (RCB0007, RIKEN Cell Bank, JPN), and HepG2 (RCB1648, RIKEN Cell Bank, JPN). In the case of human MSCs, cells were maintained in commercialized Human Mesenchymal Cell Basal Medium (MSCGM BulletKit PT-3001, Cambrex BioScience Walkersville, Inc., MD). In case of HEK293, NIH3T3-3, HeLa and HepG2, cells were maintained in Dulbecco's Modified Eagle's Medium (DMEM, high glucose 4.5 g/L with L-Glutamine and sodium pyruvate; 14246-25, Nakalai Tesque, JPN) with 10% fetal bovine serum (FBS, 29-167-54, Lot No. 2025F, Dainippon Pharmaceutical CO., LTD., JPN). All cells were cultivated in a controlled incubator at 37° C. in 5% CO₂. In experiments involving hMSCs, we used hMSCs of less than five passages, in order to avoid phenotypic changes.

(Plasmids and Transfection Reagents)

To evaluate the efficiency of transfection, the pEGFP-N1 and pDsRed2-N1 vectors (cat. no. 6085-1, 6973-1, BD Biosciences Clontech, CA) were used. Both genes' expressions were under the control of cytomegalovirus (CMV) promoter. Transfected cells continuously expressed EGFP or DsRed2, respectively. Plasmid DNAs were amplified using Escherichia coli, XL1-blue strain (200249, Stratagene, TX), and purified by EndoFree Plasmid Kit (EndoFree Plasmid Maxi Kit 12362, QIAGEN, CA). In all cases, plasmid DNA was dissolved in DNase and RNase free water. Transfection reagents were obtained as below: Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France), and ExGen 500 (R0511, Fermentas Inc., MD).

(Solid-Phase Transfection Array (SPTA) Production)

The detail of protocols for ‘reverse transfection’ was described in the web site, ‘Reverse Transfection Homepage’ (http:/staffa.wi.mit.edu/sabatini_public/reverse_trans fection.htm) or J. Ziauddin, D. M. Sabatini, Nature, 411, 2001, 107; and R. W. Zu, S. N. Bailey, D. M. Sabatini, Trends in Cell Biology, Vol. 12, No. 10, 485. In our solid phase transfection (SPTA method), three types of glass slides were studied (silanized glass slides; APS slides, and poly-L-lysine coated glass slides; PLL slides, and MAS coated slides; Matsunami Glass, JPN) with a 48 square pattern (3 mm×3 mm) separated by a hydrophobic fluoride resin coating.

(Plasmid DNA Printing Solution Preparation)

Two different ways to produce a SPTA were developed. The main differences reside in the preparation of the plasmid DNA printing solution.

(Method A)

In the case of using Effectene Transfection Reagent, the printing solution contained plasmid DNA and cell adhesion molecules (bovine plasma fibronectin (cat. no. 16042-41, Nakalai Tesque, JPN), dissolved in ultra-pure water at a concentration of 4 mg/mL). The above solution was applied on the surface of the slide using an inkjet printer (synQUAD™, Cartesian Technologies, Inc., CA) or manually, using a 0.5 to 10 μL tip. This printed slide was dried up over 15 minutes at room temperature in a safety-cabinet. Before transfection, total Effectene reagent was gently poured on the DNA-printed glass slide and incubated for 15 minutes at room temperature. The excess Effectene solution was removed from the glass slide using a vacuum aspirator and dried up at room temperature for 15 minutes in a safety-cabinet. The DNA-printed glass slide obtained was set in the bottom of a 100-mm culture dish and approximately 25 mL of cell suspension (2 to 4×10⁴ cells/mL) was gently poured into the dish. Then, the dish was transferred to the incubator at 37° C. in 5% CO₂ and incubated for 2 or 3 days.

(Method B)

In case of other transfection reagents (TransFast™, Tfx™-20, SuperFect, PolyFect, LipofectAMINE 2000, JetPEI (×4) conc., or ExGen), plasmid DNA, fibronectin, and the transfection reagent were mixed homogeneously in a 1.5-mL micro-tube according to the ratios indicated in the manufacturer's instructions and incubated at room temperature for 15 minutes before printing on a chip. The printing solution was applied onto the surface of the glass-slide using an inkjet printer or a 0.5- to 10-μL tip. The printed glass-slide was completely dried up at room temperature over 10 minutes in a safety-cabinet. The printed glass-slide was placed in the bottom of a 100-mm culture dish and approximately 3 mL of cell suspension (2 to 4×10⁴ cells/mL) was added and incubated at room temperature over 15 minutes in a safety-cabinet. After incubation, fresh medium was poured gently into the dish. Then, the dish was transferred to an incubator at 37° C. in 5% CO₂ and incubated for 2 to 3 days. After incubation, using fluorescence microscopy (IX-71, Olympus PROMARKETING, INC., JPN), we observed the transfectants, based on their expression of enhanced fluorescent proteins (EFP, EGFP and DsRed2). Phase contrast images were taken with the same microscope. In both protocols, cells were fixed by using a paraformaldehyde (PFA) fixation method (4% PFA in PBS, treatment time was 10 minutes at room temperature).

(Laser Scanning and Fluorescence Intensity Quantification)

In order to quantify the transfection efficiency, we used a DNA micro-array scanner (GeneTAC UC4×4, Genomic Solutions Inc., MI). The total fluorescence intensity (arbitrary units) was measured, and thereafter, the fluorescence intensity per surface area was calculated.

(Results)

(Fibronectin-Supported Localized Transfection)

A transfection array chip was constructed as shown in FIG. 11. The transfection array chip was constructed by microprinting a cell cultivation medium solution containing fibronectin and DNA/transfection reagent onto a poly L lysine (PLL) coated glass slide.

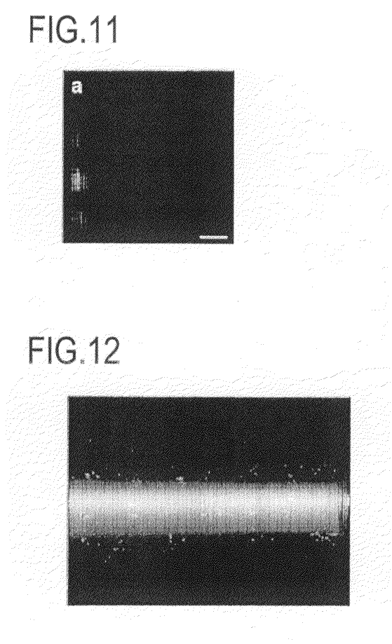

Various cells were used for this example. The cells were cultivated under typical cell cultivation conditions. As they adhered to the glass slide, the cells efficiently incorporated and expressed the genes corresponding to the DNA printed at a given position on the array. As compared to conventional transfection methods (e.g., cationic lipid or cationic polymer-mediated transfection), the efficiency of transfection using the method of the present invention was high in all the cells tested. Importantly, it was found that tissue stem cells, such as HepG2 and hMSC, which were conventionally believed to resist transfection, were efficiently transfected. hMSC was transfected at an efficiency 40 or more times higher than that of conventional techniques. In addition, high spatial localization, which is required for high-density arrays, was achieved (low cross contamination between adjacent spots on the array). This was confirmed by production of a checkered pattern array of EGFP and Ds-Red. hMSC cultivated on this array expressed the corresponding fluorescent proteins with virtually total space resolution. The result is shown in FIG. 12. As can be seen from FIG. 12, it was found that there was little cross contamination. Based on the study of the role of the individual components of the printed mixture, transfection efficiency can be optimized.

(Solid-Phase Transfection Array of Human Mesenchymal Stem Cells)

The capacity of human Mesenchymal. Stem Cells (hMSC) to differentiate into various kinds of cells is particularly intriguing in studies which target tissue regeneration and renewal. In particular, the genetic analysis of transformation of these cells has attracted attention with expectation of understanding of an agent that controls the pluripotency of hMSC. In conventional hMSC studies, it is not possible to perform transfection with desired genetic materials.

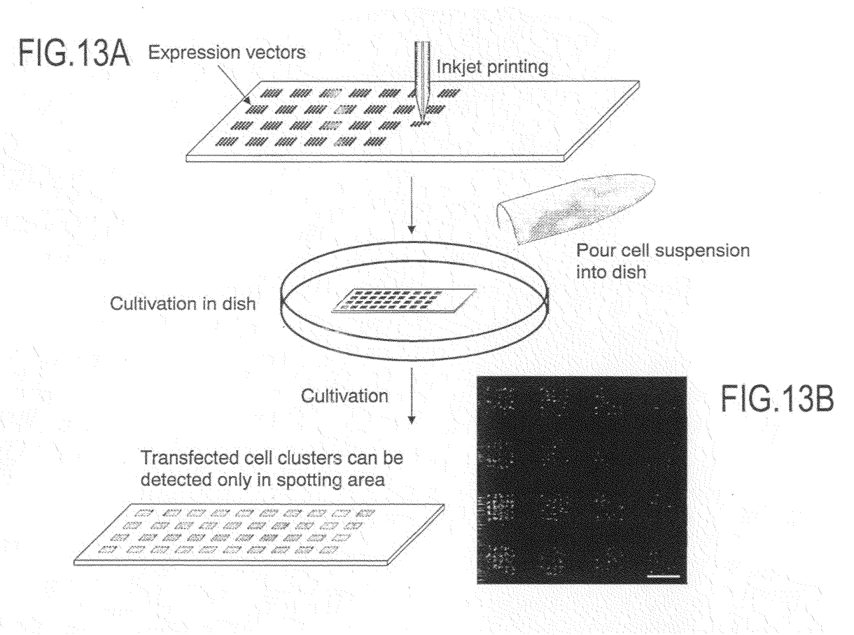

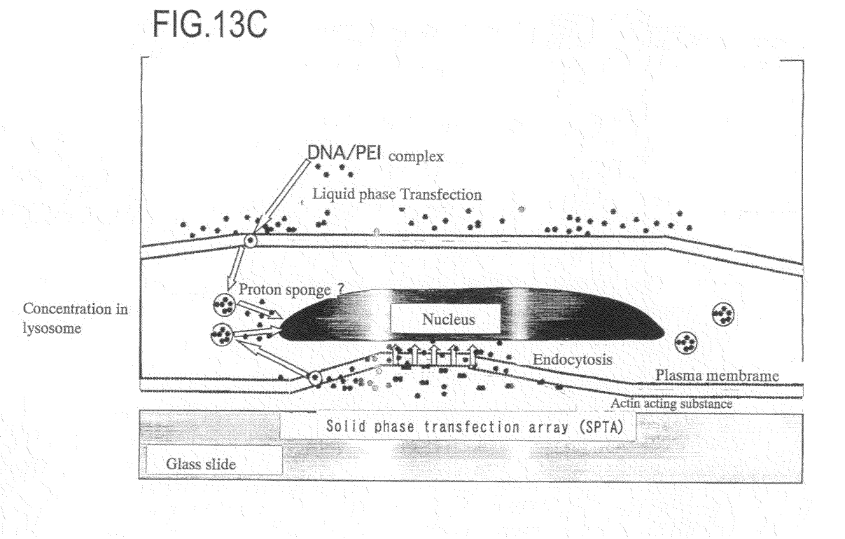

To achieve this, conventional methods include either a viral vector technique or electroporation. The present inventors developed a complex-salt system, which could be used to achieve solid phase transfection which makes it possible to obtain high transfection efficiency to various cell lines (including hMSC) and special localization in high-density arrays. An outline of solid phase transfection is shown in FIG. 13A.

It was demonstrated that solid phase transfection can be used to achieve a “transfection patch” capable of being used for in vivo gene delivery and a solid phase transfection array (SPTA) for high-throughput genetic function research on hMSC.

Although a number of standard techniques are available for transfecting mammalian cells, it is known that it is inconvenient and difficult to introduce genetic material into hMSC as compared with cell lines, such as HEK293, HeLa, and the like. Conventional viral vector delivery and electroporation techniques are each important. However, these techniques have the following inconveniences: potential toxicity (for the virus technique); difficulty in high-throughput analysis at the genomic scale; and limited applications in in vivo studies (for electroporation).

The present inventors developed solid phase support fixed system which can be easily fixed to a solid phase support and has sustained-release capability and cell affinity, whereby most of the above-described drawbacks could be overcome.

An example of the results of the above-described experiment is shown in FIG. 13B. The present inventors used our microprinting technique to fix a mixture of a selected genetic material, a transfection reagent, an appropriate cell adhesion molecule, and a salt onto a solid support. By culturing cells on a support having such a mixture fixed thereonto, the gene contained in the mixture was allowed to be taken in by the cultured cells. As a result, it became possible to allow support-adherent cells to take in DNA spatially separated therefrom (FIG. 13B).

As a result of this example, several important effects were achieved: high transfection efficiency (thereby making it possible to study a group of cells having a statistically significant scale); low cross contamination between regions having different DNA molecules (thereby making it possible to study the effects of different genes separately); the extended survival of transfected cells; high-throughput, compatible and simple detecting procedure. SPTA having these features serves as an appropriate basis for further studies.

To achieve the above-described objects, the present inventors studied five different cell lines (HEK293, HeLa, NIH3T3, HepG2 and hMSC) as described above with both our methodology (transfection in a solid phase system) (see FIGS. 13A and 13C) and conventional liquid-phase transfection under a series of transfection conditions. Cross contamination was evaluated for both systems as follows. In the case of SPTA, we printed DNA's encoding a red fluorescent protein (RFP) and a green fluorescent protein (GFP) on glass supports in a checked pattern. In the case of experiments including conventional liquid phase transfection (where cells to be transfected cannot be spatially separated from one another spontaneously), a DNA encoding GFP was used. Several transfection reagents were evaluated: four liquid transfection reagents (Effectene, TransFast™, Tfx™-20, LopofectAMiNE 2000), two polyamine (SuperFect, PolyFect), and two polyimine (JetPEI (×4) and ExGen 500).

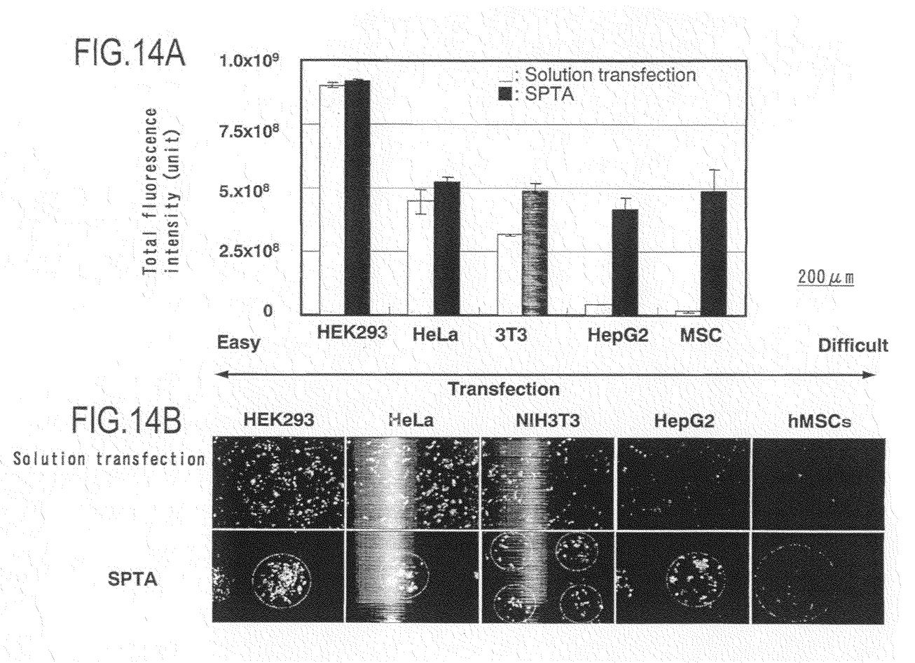

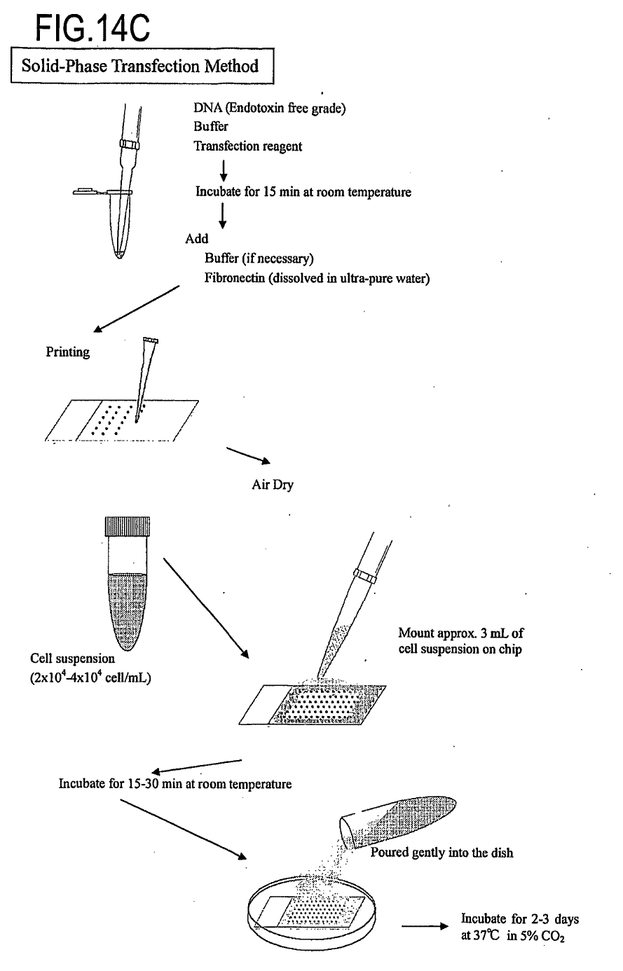

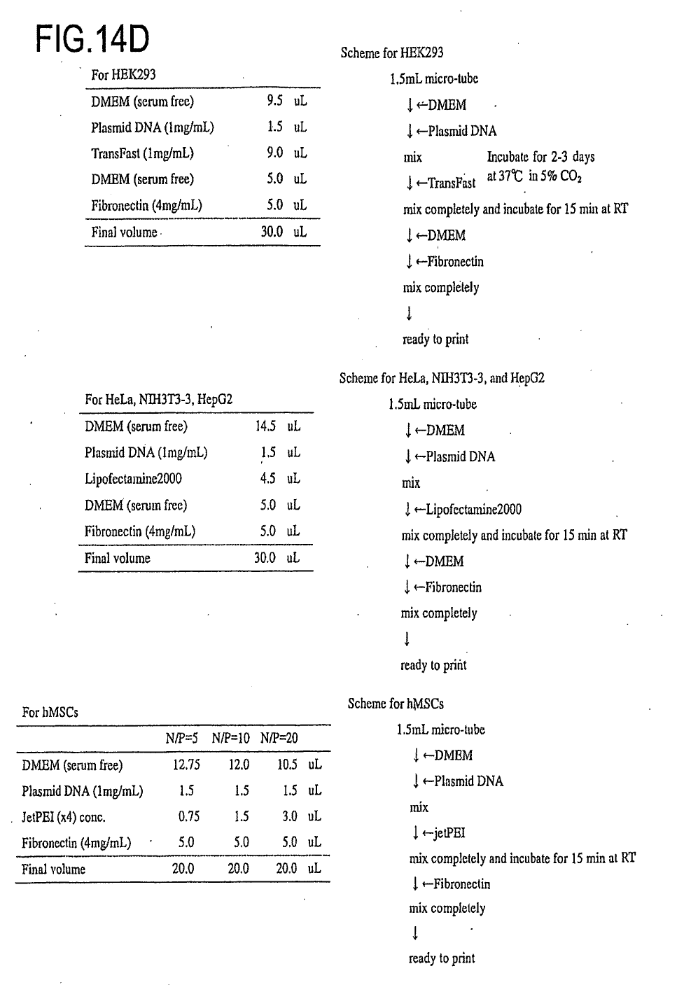

Transfection efficiency: transfection efficiency was determined as total fluorescence intensity per unit area (FIG. 14A and FIG. 14B (images)). The results of liquid phase optimal to cell lines used were obtained using different transfection reagents (see FIGS. 14C to 14D). Next, these efficient transfection reagents were used to optimize a solid phase protocol. Several tendencies were observed. For cell lines which are readily transfectable (e.g., HEK293, HeLa, NIH3T3, etc.), the transfection efficiency observed in the solid phase protocol was slightly superior to, but essentially similar to, that of the standard liquid phase protocol (FIG. 14).

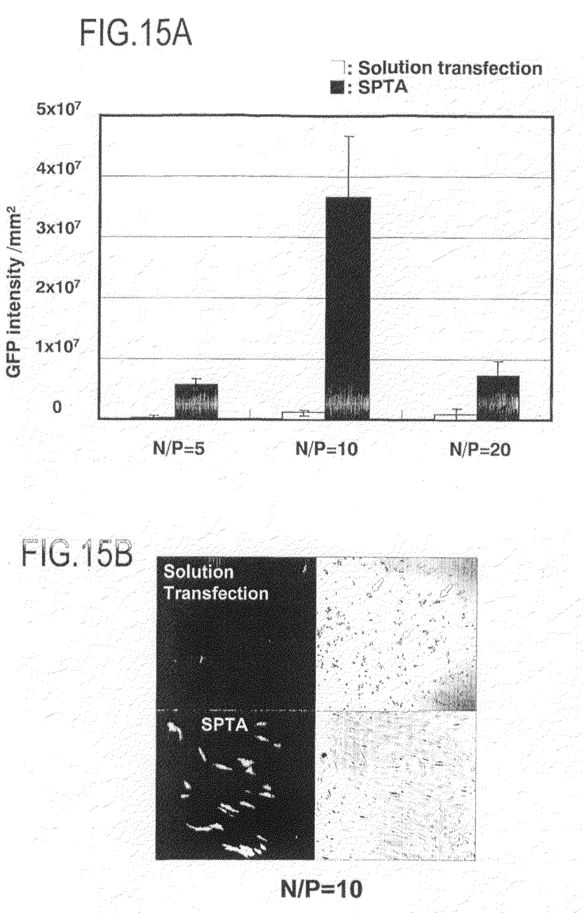

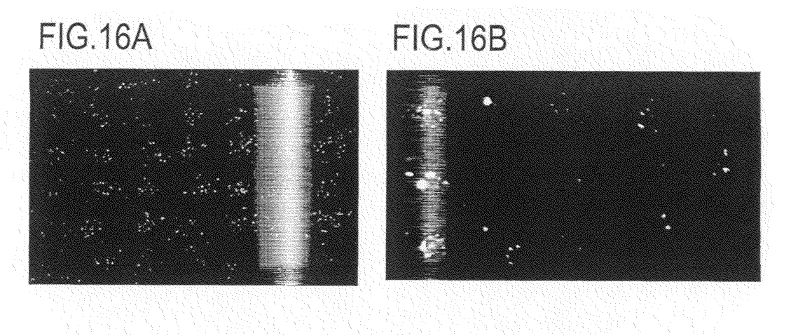

However, for cells which are difficult to transfect (e.g., hMSC, HepG2, etc.), we observed that transfection efficiency was increased up to 40 fold while the features of the cells were retained under conditions optimized to the SPTA methodology (see the above-described protocol and FIGS. 14C and 14D). In the case of hMSC (FIG. 15), the best conditions included use of a polyethylene imine (PEI) transfection reagent. As expected, important factors for achieving high transfection efficiency are the charge balance (N/P ratio) between the number of nitrogen atoms (N) in the polymer and the number of phosphate residues (P) in plasmid DNA and DNA concentration. Generally, increases in the N/P ratio and the concentration lead to an increase in transfection efficiency. We also observed a significant reduction in the survival rate of hMSC cells in liquid phase transfection experiments where the DNA concentration was high and the N/P ratio was high. Because of these two opposing factors, the liquid phase transfection of hMSC had a relatively low cell survival rate (N/P ratio >10). In the case of the SPTA protocol, however, a considerably high N/P ratio (fixed to the solid support) and DNA concentration were tolerable (probably attributed to the effect of the solid support stabilizing cell membrane) while the cell survival rate and the cellular state were not significantly affected. Therefore, this is probably responsible for the dramatic improvement in transfection efficiency. It was found that the N/P ratio of 10 was optimal for SPTA, and a sufficient transfection level was provided while minimizing cytotoxicity. Another reason for the increase in transfection efficiency observed in the case of the SPTA protocol is that a high local ratio of the DNA concentration to the transfection reagent concentration was achieved (this leads to cell death in liquid phase transfection experiments).

A coating agent used is crucial for the achievement of high transfection efficiency on chips. It was found that when a glass chip is used, PLL provided best results both for transfection efficiency and cross contamination (described below). When fibronectin coating was not used, few transfectants were observed (all the other experimental conditions were retained unchanged). Although not completely established, fibronectin probably plays a role in accelerating cell adhesion process (data not shown), and thus, limiting the time which permits the diffusion of DNA released from the surface.

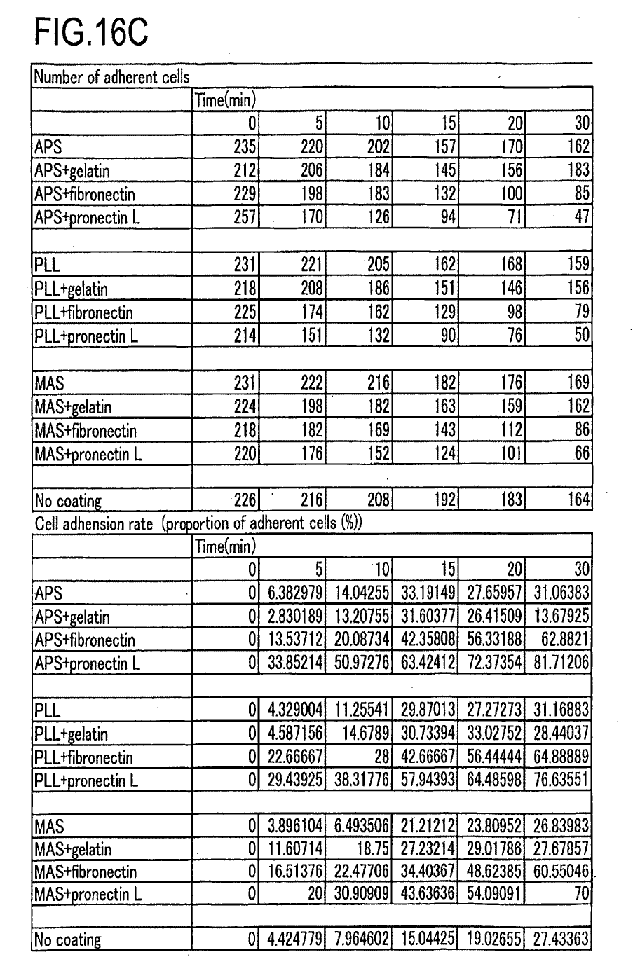

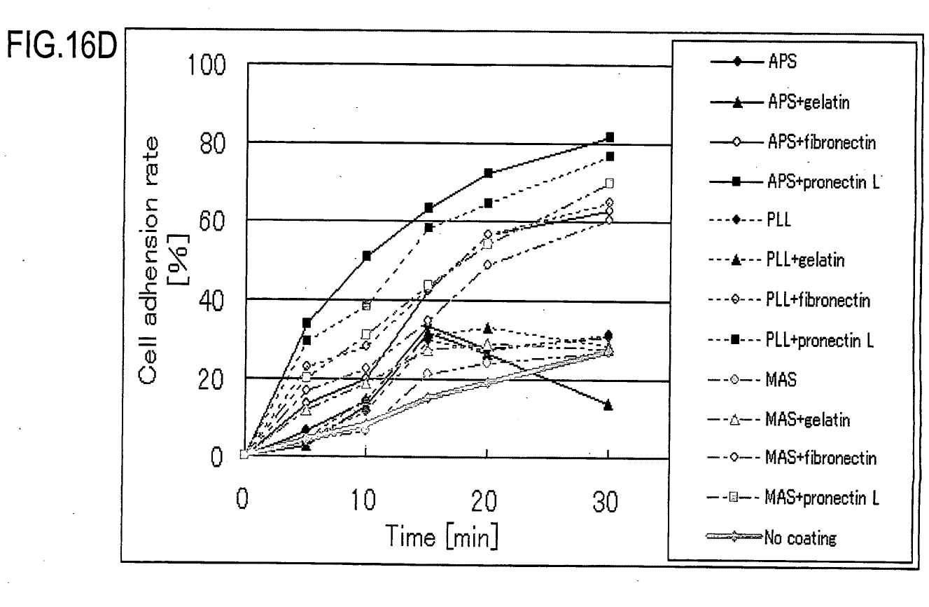

Low cross contamination: apart from the higher transfection efficiency observed in the SPTA protocol, an important advantage of the technique of the present invention is to achieve an array of separated cells, in which selected genes are expressed in the separate positions. The present inventors printed JetPEI (see the “Experimental protocols” section) and two different reporter genes (RFP and GFP) mixed with fibronectin on glass surface coated with fibronectin. The resultant transfection chip was subjected to appropriate cell culture. Expressed GFP and RFP were localized in regions, in which corresponding cDNA had been spotted, under experimental conditions which had been found to be best. Substantially no cross contamination was observed (FIG. 16). In the absence of fibronectin or PLL, however, cross contamination which hinders solid phase transfection was observed, and the transfection efficiency was significantly lower (see FIG. 6). This result demonstrated the hypothesis that the relative proportion of plasmid DNA, which was released from the cell adhesion and the support surface, is a factor important for high transfection efficiency and high cross contamination.

Another cause of cross contamination may be the mobility of transfected cells on a solid support. The present inventors measured both the rate of cell adhesion (FIG. 16C) and the diffusion rate of plasmid DNA on several supports. As a result, substantially no DNA diffusion occurred under optimum conditions. However, a considerably amount of plasmid DNA were diffused under high cross contamination conditions until cell adhesion was completed, so that plasmid DNA was depleted from the solid phase surface.

This established technique is of particular importance in the context of cost-effective high-throughput gene function screening. Indeed, the small amounts of transfection reagent and DNA required, as well as the possible automatization of the entire process (from plasmid isolation to detection) increase the utility of the above presented method.

In conclusion, the present invention successfully realized a hMSC transfection array in a system using complex-salt. With this technique, it will be possible to achieve high-throughput studies using the solid phase transfection, such as the elucidation of the genetic mechanism for differentiation of pluripotent stem cells. The detailed mechanism of the solid phase transfection as well as methodologies for the use of this technology for high throughput, real time gene expression monitoring can be applied for various purposes.

### Example 5

### RNAi Transfection Microarray

Arrays were produced as described in the above-described example. As genetic material, mixtures of plasmid DNA (pDNA) and shRNA were used. The compositions of the mixtures are shown in Table 2.

TABLE 2

| | pDNA vs. shRNA ratio [μL/μL] | | | | |
|---|---|---|---|---|---|
| | 9:1 | 7:3 | 1:1 | 3:7 | 1:9 |
| pEGFP-N1 (1 mg/mL) | 1.8 | 1.4 | 1.0 | 0.6 | 0.2 |
| pPUR6iGFP272 (1 mg/mL) | 0.2 | 0.6 | 1.0 | 1.4 | 1.8 |
| pDsRed2-1 (1 mg/mL) | 0.2 | 0.6 | 1.0 | 1.4 | 1.8 |
| Lipofectamine2000 | 4.0 | 4.0 | 4.0 | 4.0 | 4.0 |
| Fibronectin (4 mg/mL) | 5.0 | 5.0 | 5.0 | 5.0 | 5.0 |

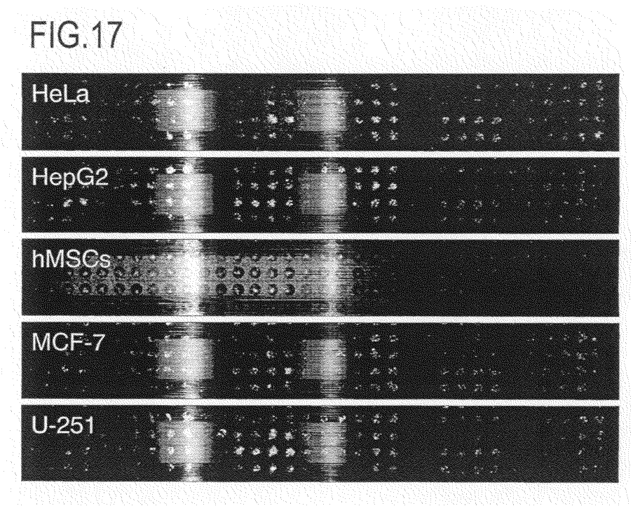

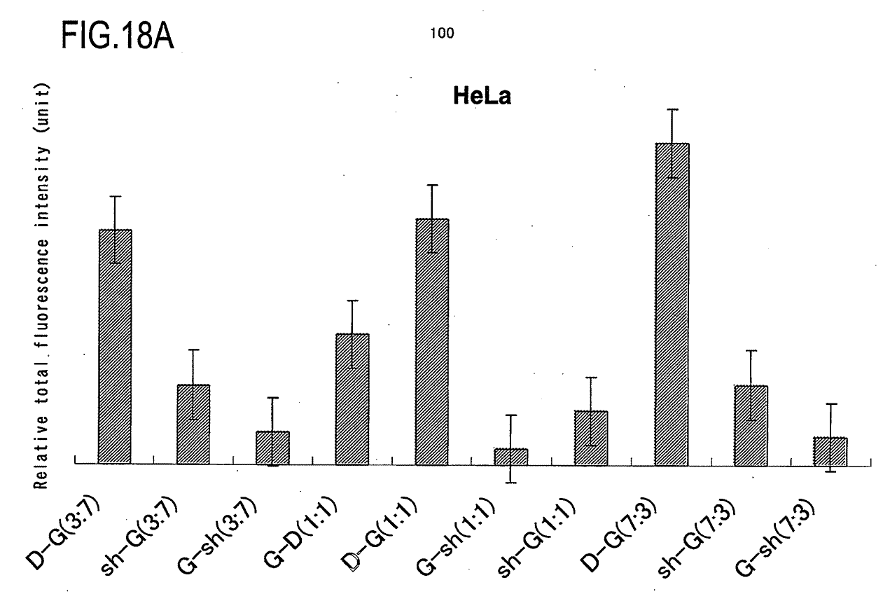

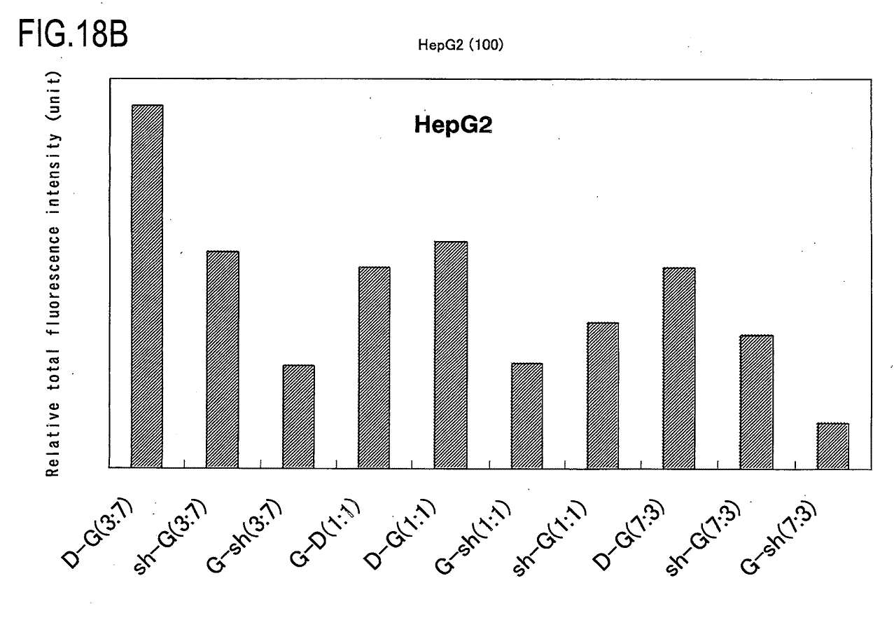

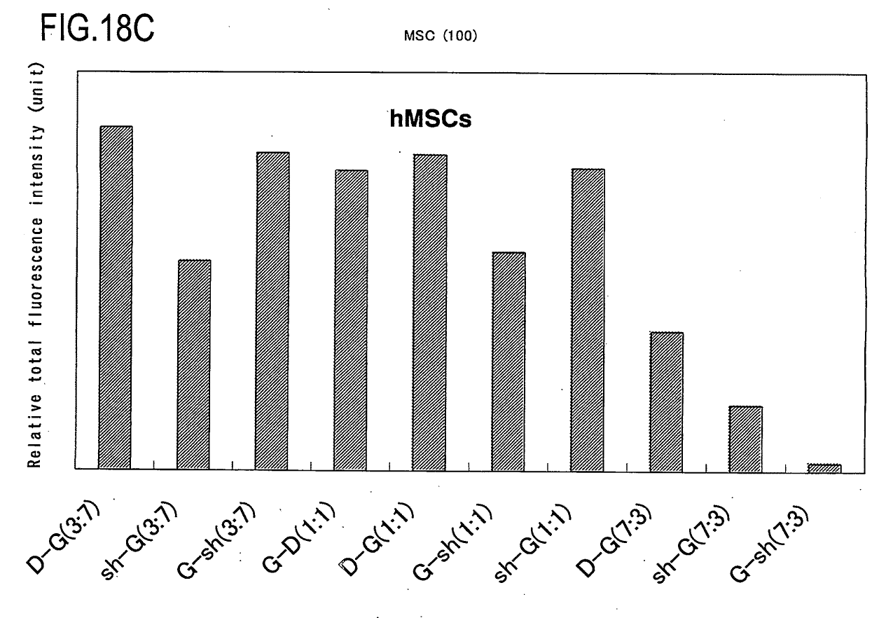

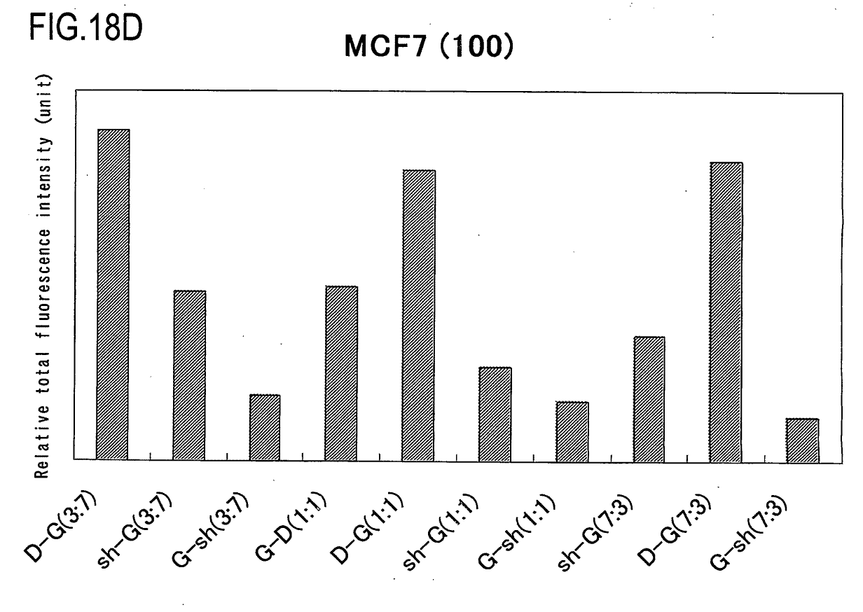

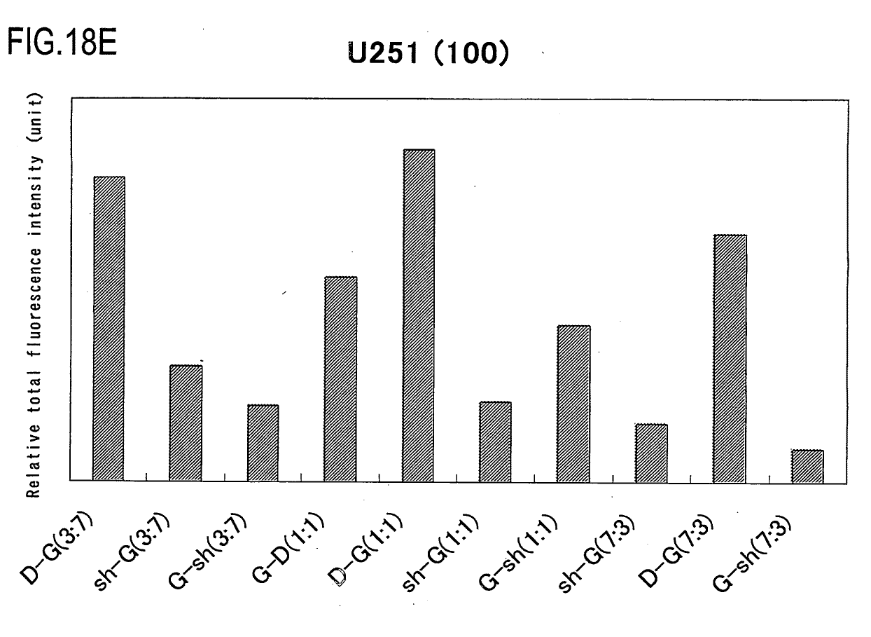

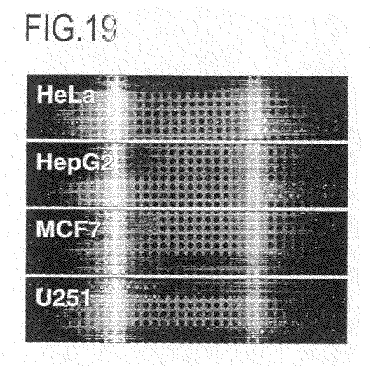

The results are shown in FIG. 17. For each of the 5 cells, the results of FIG. 17 are converted into numerical data in FIGS. 18A to 18E.

Thus, it was revealed that the method of the present invention is applicable to any cells.

### Example 6

### Use of RNAi Microarray=siRNA

Next, siRNA was used instead of shRNA to construct RNAi transfection microarrays in accordance with a protocol as described in the above-described example.

18 transcription factor reporters and actin promoter vectors described in Table 3 were used to synthesize 28 siRNAs for the transcription factors. siRNA for EGFP was used as a control. Each siRNA was evaluated as to whether or not it knocks out a target transcription factor. Scramble RNAs were used as negative controls, and their ratios were evaluated.

TABLE 3

Mercury signaling pathway
pAP1(PMA)-EGFP
pAP1-EGFP
pCRE-EGFP
pERE-EGFP
pE2F-EGFP
pGAS-EGFP
pGRE-EGFP
pHSE-EGFP
pISRE-EGFP
pMyc-EGFP
pNFAT-EGFP
pNFkB-EGFP
p53-EGFP
pRARE-EGFP
pRb-EGFP
pSRE-EGFP
pSTAT3-EGFP
pTRE-EGFP

Each cell was subjected to solid phase transfection, followed by culture for two days. Images were taken using a fluorescence image scanner, and the fluorescent level was quantified.

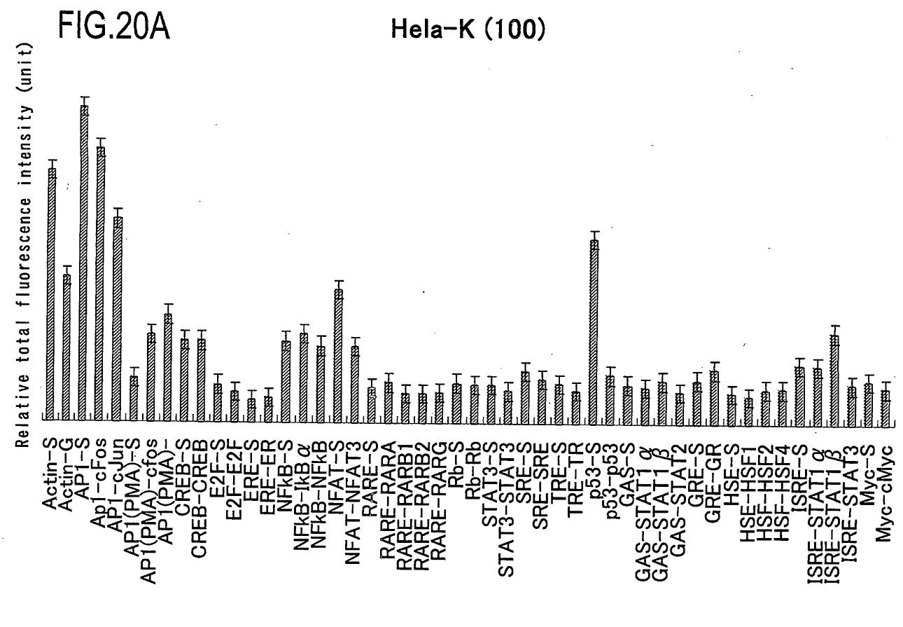

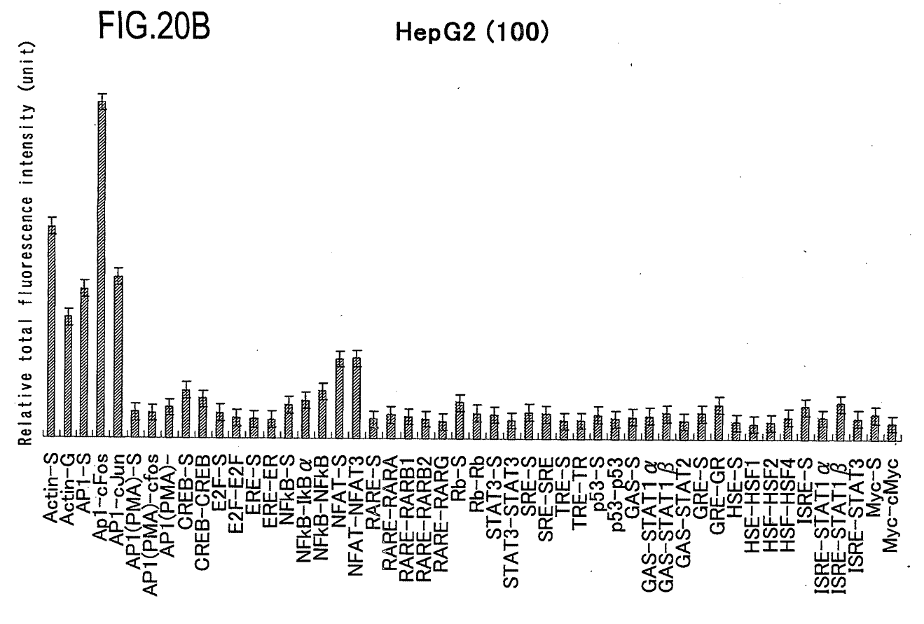

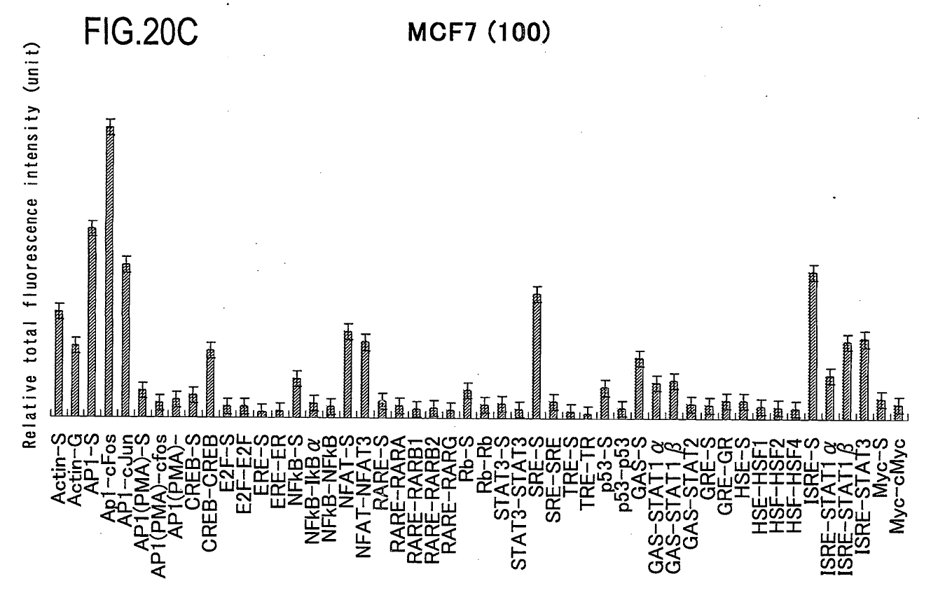

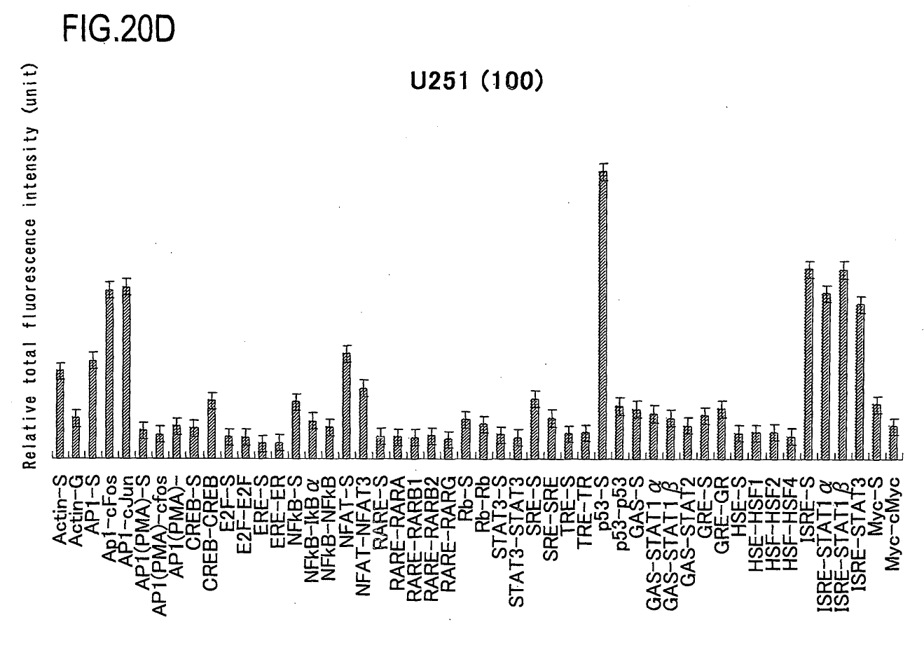

The results are shown in FIG. 19. The results were summarized for each gene in FIGS. 20A to 20D.

As shown in FIGS. 19 and 20A to 20D, when RNAi was used, the expression of each gene was specifically suppressed. Thus, it was demonstrated that an array having a plurality of genetic materials, which is applicable to RNAi, can be realized and time-lapse analysis can be performed for the effect of RNAi on cells.

### Example 7

### Transfection Array Using PCR Fragments

Next, it was demonstrated that the present invention could be implemented when PCR fragments were used as genetic materials. The procedure will be described below.

PCR was performed to obtain nucleic acid fragments as shown in FIG. 21. These fragments were used as genetic materials which were applied to transfection microarrays. The procedure will be described below.

PCR primers were:

(SEQ ID NO.: 12)

GG ATAACCGTAT TACCGCCATG CAT;
and

(SEQ ID NO.: 13)

ccctatctcggtctattcttttg CAAAAGAATA GACCGAGATA GGG.

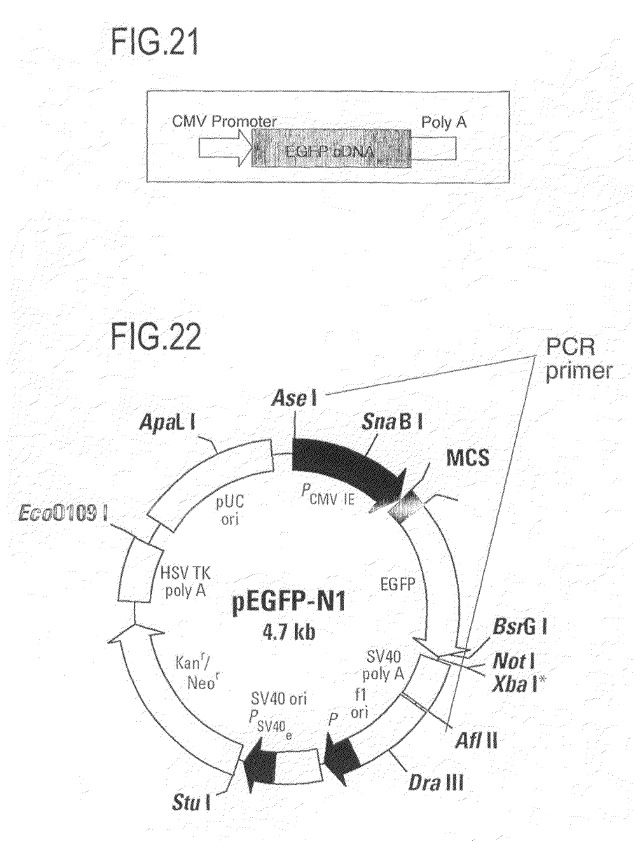

pEGFP-N1 (see FIG. 22) was used as a template.

PCR conditions were described in Table 4 below.

TABLE 4

| | | |
|---|---|---|
| Distilled water | 33.5 | μL |
| 10 × KOD-Plus-buffer | 5 | μL |
| 2 mM dNTPs | 5 | μL |
| 25 mM MgSO₄ | 2 | μL |
| Primer (10 μM each) | 1.5 | μL |
| Template DNA (1 ng) | 2 | μL |
| KOD-Plus-(1 unit/uL) | 1 | μL |
| Total | 50 | μL |

Cycle conditions: 94° C., 2 min→(94° C., 15 sec→60° C., 30 sec→68° C., 3 min)→4° C. (the process in parenthesis was performed 30 times)

The resultant PCR fragment was purified with phenol/chloroform extraction and ethanol precipitation. The PCR fragment has the following sequence:

(SEQ ID NO.: 14)

GG ATAACCGTAT TACCGCCATG CAT TAGTTATTAA TAGTAATCAA
TTACGGGGTC ATTAGTTCAT AGCCCATATA TGGAGTTCCG
CGTTACATAA CTTACGGTAA ATGGCCCGCC TGGCTGACCG
CCCAACGACC CCCGCCCATT GACGTCAATA ATGACGTATG
TTCCCATAGT AACGCCAATA GGGACTTTCC ATTGACGTCA
ATGGGTGGAG TATTTACGGT AAACTGCCCA CTTGGCAGTA
CATCAAGTGT ATCATATGCC AAGTACGCCC CCTATTGACG
TCAATGACGG TAAATGGCCC GCCTGGCATT ATGCCCAGTA
CATGACCTTA TGGGACTTTC CTACTTGGCA GTACATCTAC
GTATTAGTCA TCGCTATTAC CATGGTGATG CGGTTTTGGC
AGTACATCAA TGGGCGTGGA TAGCGGTTTG ACTCACGGGG
ATTTCCAAGT CTCCACCCCA TTGACGTCAA TGGGAGTTTG
TTTTGGCACC AAAATCAACG GGACTTTCCA AAATGTCGTA
ACAACTCCGC CCCATTGACG CAAATGGGCG GTAGGCGTGT
ACGGTGGGAG GTCTATATAA GCAGAGCTGG TTTAGTGAAC
CGTCAGATCC GCTAGCGCTA CCGGACTCAG ATCTCGAGCT
CAAGCTTCGA ATTCTGCAGT CGACGGTACC GCGGGCCCGG
GATCCACCGG TCGCCACCAT GGTGAGCAAG GGCGAGGAGC
TGTTCACCGG GGTGGTGCCC ATCCTGGTCG AGCTGGACGG
CGACGTAAAC GGCCACAAGT TCAGCGTGTC CGGCGAGGGC
GAGGGCGATG CCACCTACGG CAAGCTGACC CTGAAGTTCA
TCTGCACCAC CGGCAAGCTG CCCGTGCCCT GGCCCACCCT
CGTGACCACC CTGACCTACG GCGTGCAGTG CTTCAGCCGC
TACCCCGACC ACATGAAGCA GCACGACTTC TTCAAGTCCG
CCATGCCCGA AGGCTACGTC CAGGAGCGCA CCATCTTCTT
CAAGGACGAC GGCAACTACA AGACCCGCGC CGAGGTGAAG
TTCGAGGGCG ACACCCTGGT GAACCGCATC GAGCTGAAGG
GCATCGACTT CAAGGAGGAC GGCAACATCC TGGGGCACAA
GCTGGAGTAC AACTACAACA GCCACAACGT CTATATCATG
GCCGACAAGC AGAAGAACGG CATCAAGGTG AACTTCAAGA
TCCGCCACAA CATCGAGGAC GGCAGCGTGC AGCTCGCCGA
CCACTACCAG CAGAACACCC CCATCGGCGA CGGCCCCGTG
CTGCTGCCCG ACAACCACTA CCTGAGCACC CAGTCCGCCC
TGAGCAAAGA CCCCAACGAG AAGCGCGATC ACATGGTCCT
GCTGGAGTTC GTGACCGCCG CCGGGATCAC TCTCGGCATG
GACGAGCTGT ACAAGTAAAG CGGCCGCGAC TCTAGATCAT
AATCAGCCAT ACCACATTTG TAGAGGTTTT ACTTGCTTTA
AAAAACCTCC CACACCTCCC CCTGAACCTG AAACATAAAA
TGAATGCAAT TGTTGTTGTT AACTTGTTTA TTGCAGCTTA
TAATGGTTAC AAATAAAGCA ATAGCATCAC AAATTTCACA
AATAAAGCAT TTTTTTCACT GCATTCTAGT TGTGGTTTGT
CCAAACTCAT CAATGTATCT TAAGGCGTAA ATTGTAAGCG
TTAATATTTT GTTAAAATTC GCGTTAAATT TTTGTTAAAT
CAGCTCATTT TTTAACCAAT AGGCCGAAAT CGGCAAAATC
CCTTATAAAT CAAAAGAATA GACCGAGATA GGG.

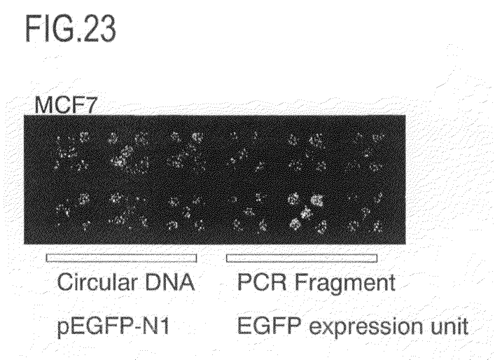

Chips were produced using the PCR fragment. MCF7 was disseminated on the chips. After two days, images were obtained using a fluorescence image scanner. The results are shown in FIG. 23. In FIG. 23, the PCR fragment is compared with circular DNA. In either case, transfection was successful. It was revealed that the PCR fragment, which was used as a genetic material, could be transfected into cells, as with full-length plasmids, so that time-lapse analysis could be performed for the cells. Thus, the fixing effect of the salt and the enhancement of gene introduction by such an effect were confirmed.

### Example 8

### Type of Support

Next, when a solid phase support is made of silica, silicon, a ceramic, silicon dioxide, or a plastic instead of glass, it is determined whether or not a similar effect of actin acting substances is observed.

These materials are available from Matsunami Glass. Arrays are produced as described above.

As a″result, it is revealed that a similar effect of actin can be observed for the material used.

### Example 9

### Regulation of Gene Expression Using Tetracycline-Dependent Promoter

As described in the above-described examples, it was demonstrated that a tetracycline-dependent promoter could be used to produce a profile showing how gene expression is regulated. The sequences described below were used.

As the tetracycline-dependent promoter (and its gene vector construct), pTet-Off and pTet-On vectors (BD Biosciences) were used (see http://www.clontech.com/techinfo/vectors/cattet.shtml). As a vector, pTRE-d2EGFP (SEQ ID NO.: 18) was used (see http://www.clontech.com/techinfo/vectors/vectorsT-Z/pTR E-d2EGFP.shtml).

pTet-Off (BD Clonetech K1620-A)

Fragment containing P_{CMV}: 86-673

Tetracycline-responsive transcriptional activator (tTA): 774-1781

Col El origin of replication: 2604-3247

Ampicillin resistance gene:
β-lactamase coding sequences: 4255-3395

Fragment containing the SV40 poly A signal: 1797-2254

Neomycin/kanamycin resistance gene: 6462-5668

SV40 promoter (P_{SV40}) controlling expression of neomycin/kanamycin resistance gene: 7125-6782.

pTet-ON(BD Clonetech K1621-A)

Fragment containing P_{CMV}: 86-673

Reverse tetracycline-responsive transcriptional activator (rtTA): 774-1781

pUC origin of replication: 2604-3247

Ampicillin resistance gene:
β-lactamase coding sequences: 4255-3395

Fragment containing the SV40 poly A signal: 1797-2254

Neomycin/kanamycin resistance gene: 6462-5668

SV40 promoter (P_{SV40}) controlling expression of neomycin/kanamycin resistance gene: 7125-6782.

pTRE-d2EGFP(BD Clonetech 6242-1)

P_{hCMV*-1} Tet-responsive promoter: 1-438
Tet-responsive element (TRE): 1-318
Location of seven tetO18-mers: 15-33; 57-75; 99-117; 141-159; 183-201; 225-243; & 257-275
Fragment containing P_{minCMV}: 319-438
TATA box 341-348

Destabilized enhanced green fluorescent protein (d2EGFP) gene
Start codon: 445-447; stop codon: 1288-1290
Insertion of Val at position #2: 448-450
GFPmut1 mutations (Phe-64-Leu, Ser-65-Thr): 634-639
His-231-Leu: 1137
Mouse ornithine decarboxylase (MODC) PEST sequence: 1167-1290

Fragment containing SV40 poly A signal: 1330-1787
(approximate coordinates of poly A signal: 1448-1453)

Fragment containing Col El origin of replication: 2137-2780

Ampicillin resistance gene
β-lactamase coding sequences: 2928-3788
start codon: 3788-3786
stop codon: 2928-2930

(Protocol)

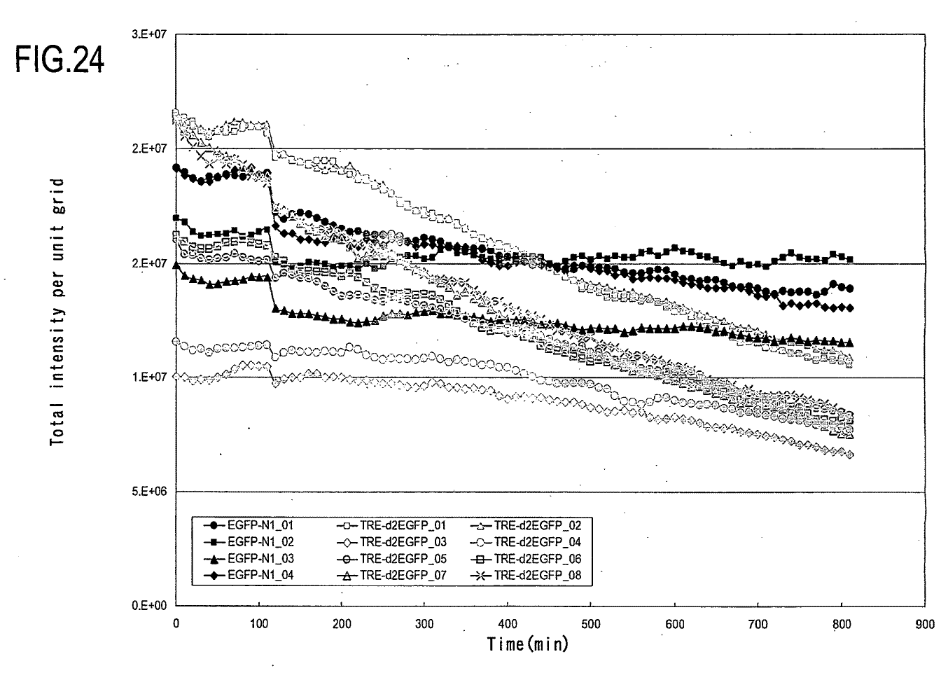

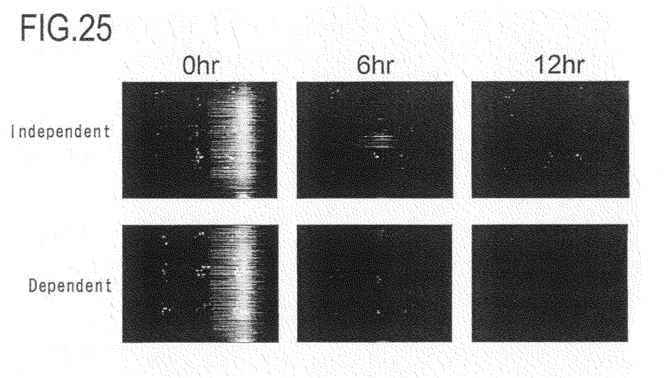

pTet-Off and pTet-On (SEQ ID NOS.: 15 and 16, respectively) were printed onto array substrates. Real time measurement was performed on the array substrates to determine whether or not tetracycline regulates gene expression. The results are shown in FIG. 24. As shown in FIG. 24, a change in gene expression was detected only for the tetracycline-dependent promoter. FIG. 25 is a photograph showing the actual states of expression for the tetracycline-dependent promoter and the tetracycline-independent promoter. As can be seen, the difference between them is measurable by the naked eye.

Although certain preferred embodiments have been described herein, it is not intended that such, embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

According to the present invention, transfection efficiency could be increased either in a solid phase and in a liquid phase. The reagent for increasing transfection efficiency is useful for transfection in, particularly, solid phases.

1. A composition for increasing the efficiency of introducing a target substance into a cell, comprising:
(a) an actin acting substance.

2. A composition according to claim 1, wherein the actin acting substance may be an extracellular matrix protein or a variant or fragment thereof.

3. A composition according to claim 2, wherein the actin acting substance comprises at least one protein selected from the group consisting of fibronectin, laminin, and vitronectin, or a variant or fragment thereof.

4. A composition according to claim 1, wherein the actin acting substance comprises:
(a-1) a protein molecule comprising at least amino acids 21 to 241 of SEQ ID NO.: 11 constituting an Fn1 domain, or a variant thereof;
(a-2) a protein molecule having an amino acid sequence set forth in SEQ ID NO.: 2 or 11, or a variant or fragment thereof;
(b) a polypeptide having an amino acid sequence set forth in SEQ ID NO.: 2 or 11 having at least one mutation selected from the group consisting of at least one amino acid substitution, addition, and deletion, and having a biological activity;
(c) a polypeptide encoded by a splice or alleic mutant of a base sequence set forth in SEQ ID NO.: 1;
(d) a polypeptide being a species homolog of the amino acid sequence set forth in SEQ ID NO.: 2 or 11; or
(e) a polypeptide having an amino acid sequence having at least 70% identity to any one of the polypeptides (a-1) to (d), and having a biological activity.

5. A composition according to claim 1, wherein the Fn1 domain comprises amino acids 21 to 577 of SEQ ID NO.: 11.

6. A composition according to claim 1, wherein the protein molecule having the Fn1 domain is fibronectin or a variant or fragment thereof.

7. A composition according to claim 1, further comprising a gene introduction reagent.

8. A composition according to claim 1, wherein the gene introduction reagent is selected from the group consisting of cationic polymers, cationic lipids, and calcium phosphate.

9. A composition according to claim 1, further comprising a particle.

10. A composition according to claim 9, wherein the particle comprises gold colloid.

11. A composition according to claim 1, further comprising a salt.

12. A composition according to claim 11, wherein the salt is selected from the group consisting of salts contained in buffers and salts contained in media.

13. A kit for increasing the efficiency of introducing a target substance into a cell, comprising:
(a) a composition comprising an actin acting substance; and
(b) a gene introduction reagent.

14. A composition for increasing the efficiency of introducing a target substance into a cell, comprising:
A) a target substance; and
B) an actin acting substance.

15. A composition according to claim 14, wherein the target substance comprises a substance selected from the group consisting of DNA, RNA, polypeptides, sugars, and complexes thereof.

16. A composition according to claim 14, wherein the target substance comprises DNA encoding a gene sequence to be transfected.

17. A composition according to claim 16, further comprising a gene introduction reagent.

18. A composition according to claim 14, wherein the actin acting substance is an extracellular matrix protein or a variant or fragment thereof.

19. A composition according to claim 14, wherein the composition is provided in liquid phase.

20. A composition according to claim 14, wherein the composition is provided in solid phase.

21. A device for introducing a target substance into a cell, comprising:
A) a target substance; and
B) an actin acting substance,
wherein the composition is fixed to a solid phase support.

22. A device according to claim 21, wherein the target substance comprises a substance selected from the group consisting of DNA, RNA, polypeptides, sugars, and complexes thereof.

23. A device according to claim 21, wherein the target substance comprises DNA encoding a gene sequence to be transfected.

24. A device according to claim 23, further comprising a gene introduction reagent.

25. A device according to claim 21, wherein the actin acting substance is an extracellular matrix protein or a variant or fragment thereof.

26. A device according to claim 21, wherein the solid phase support is selected from the group consisting of plates, microwell plates, chips, glass slides, films, beads, and metals.

27. A device according to claim 21, wherein the solid phase support is coated with a coating agent.

28. A device according to claim 27, wherein the coating agent comprises a substance selected from the group consisting of poly-L-lysine, silane, MAS, hydrophobic fluorine resins, and metals.

29. A method for increasing the efficiency of introducing a target substance into a cell, comprising the steps of:
A) providing the target substance;
B) providing an actin acting substance; and
C) contacting the target substance and the actin acting substance with the cell.

30. A method according to claim 29, wherein the target substance comprises a substance selected from the group consisting of DNA, RNA, polypeptides, sugars, and complexes thereof.

31. A method according to claim 29, wherein the target substance comprises DNA encoding a gene sequence to be transfected.

32. A method according to claim 31, further comprising providing a gene introduction reagent, wherein the gene introduction reagent is contacted with the cell.

33. A method according to claim 29, wherein the actin acting substance is an extracellular matrix protein or a variant or fragment thereof.

34. A method according to claim 29, wherein the steps are conducted in liquid phase.

35. A method according to claim 29, wherein the steps are conducted in solid phase.

36. A method for increasing the efficiency of introducing a target substance into a cell, comprising the steps of:
I) fixing a composition to a solid support, wherein the composition comprising:
A) a target substance; and
B) an actin acting substance; and
II) contacting the cell with the composition on the solid support.

37. A method according to claim 36, wherein the target substance comprises a substance selected from the group consisting of DNA, RNA, polypeptides, sugars, and complexes thereof.

38. A method according to claim 36, wherein the target substance comprises DNA encoding a gene sequence to be transfected.

39. A method according to claim 38, further comprising providing a gene introduction reagent, wherein the gene introduction reagent is contacted with the cell.

40. A method according to claim 39, further comprising forming a complex of the DNA and the gene introduction reagent after providing the gene introduction reagent, wherein after the forming step, the composition is provided by providing the actin acting substance.

41. A method according to claim 36, wherein the actin acting substance is an extracellular matrix protein or a variant or fragment thereof.
